(19) United States
(12) Patent Application Publication
(10) Pub. No.: US 2009/0137577 A1
(43) Pub. Date: May 28, 2009

(54) HETEROCYCLIC COMPOUNDS

(75) Inventors: Allen J. Duplantier, Ledyard, CT (US); Ivan Efremov, Gales Ferry, CT (US); Lei Zhang, Waterford, CT (US); Noha S. Maklad, Waterford, CT (US); Theresa O'Sullivan, Norwich, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc.

(21) Appl. No.: 12/165,056

(22) Filed: Jun. 30, 2008

Related U.S. Application Data
(60) Provisional application No. 60/947,040, filed on Jun. 29, 2007.

(51) Int. Cl. A61K 31/536; C07D 265/18; A61P 25/00

(52) U.S. Cl. 514/230.5; 544/92

(57) ABSTRACT

Compounds and pharmaceutically acceptable salts of the compounds are disclosed, wherein the compounds have the structure of Formula I

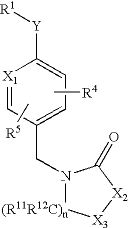

as defined in the specification. Corresponding pharmaceutical compositions, methods of treatment, methods of synthesis, and intermediates are also disclosed.

This application claims the benefit of U.S. Provisional Application No. 60/947,040, filed Jun. 29, 2007.

### FIELD OF THE INVENTION

The present invention comprises a novel class of compounds having the structure of formula I (including tautomers and salts of those compounds) and pharmaceutical compositions comprising a compound of formula I. The present invention also comprises methods of treating a subject by administering a therapeutically effective amount of a compound of formula I to the subject. These compounds are useful for the conditions disclosed herein. The present invention further comprises methods for making the compounds of formula I and corresponding intermediates.

### BACKGROUND OF THE INVENTION

The present invention provides potentiators of glutamate receptors (compounds of formula I), pharmaceutical compositions thereof, and methods of using the same, processes for preparing the same, and intermediates thereof.

Glutamate is an abundant and important neurotransmitter in mammalian CNS that is involved in a variety of normal CNS functions and has been suggested to be involved in CNS disorders. The functions of glutamate as a neurotransmitter are mediated by two families of glutamate receptors on cells in the CNS—the ionotropic glutamate receptor family, which contain integral ion channels, and the metabotropic glutamate receptor family whose members are linked to G-proteins (Ozawa et al., Prog. Neurobiol., 1998, 54, 581-618). The mGlu receptors are part of the Type III protein coupled receptor (GPCR) superfamily, which also includes the GABA-B receptors, calcium-sensing receptor, putative pheromone receptors, and taste receptors (Pin et al., Pharmacol. Ther., 2003, 98, 325-354).

A key feature in the understanding of many members of the Type III GPCR superfamily that has emerged recently is the recognition of multiple binding sites on these receptors for different classes of pharmacological agents. One class of agents bind to the extracellular endogenous ligand binding site on the receptor (the orthosteric site)—both pharmacological agonists and antagonists that bind to this site have been described for members of the Type III receptor superfamily (Conn and Pin, Ann. Rev. Pharmacol. Toxicol., 1997, 37, 205-237). More recently, for many receptors in the Type III superfamily (including multiple types of mGlu receptors), compounds have been described that bind to regions of the receptor distinct from the orthosteric site (Pin et al., Mol. Pharmacol., 2001, 60, 881-884). These are termed allosteric ligands, and for many type III receptors the discovery of allosteric ligands has provided pharmacological tools which can be differentiated in chemical structure from orthosteric ligands.

Allosteric compounds may also provide pharmacological distinctions not possible with orthosteric ligands. For example, allosteric compounds may not directly activate a receptor, but rather modulate (by enhancing or reducing) the activity of the endogenous ligand upon its binding to the orthosteric site. In addition, pharmacological distinctions include the potential for pharmacological specificity between related receptors types that share the same endogenous ligand. For example, the structural similarity of the glutamate binding site on closely related members of the mGlu receptor family has resulted in the development of agonist and antagonist compounds that bind to this site which are similar in potency toward multiple receptor within a family. There may be advantages to targeting the development of novel, selective pharmacological agents for these receptors that bind at allosteric sites, since other regions of the receptors show less homology across receptor subtypes than the glutamate binding site.

The metabotropic glutamate (mGlu) receptors include eight subtypes which have been categorized into three groups based on their structural homologies, the second messenger systems to which they are linked, and their pharmacology. The mGlu receptors are found on both CNS neurons and glia, and have been implicated in a variety of CNS functions. Because of the key role of glutamate in CNS function, pharmacological manipulation of this class of glutamate receptors has been suggested as an avenue to treat a variety of diseases (Conn and Pin, Ann. Rev. Pharmacol. Toxicol., 1997, 37, 205-237; Schoepp and Conn, Trends Pharmacol. Sci., 1993, 14, 13-20).

The present invention relates to the mGluR2 subtype of mGlu receptor, which together with mGluR3 receptors comprise the group II mGlu receptors. mGluR2 receptors have been shown to modulate synaptic transmission at both excitatory glutamate-releasing and inhibitory GABA-releasing neurons (Schoepp, J. Pharmacol. Exp. Ther., 2001, 299, 12-20). The pharmacological tools that have been used to probe the functions of mGluR2 receptors are direct agonist and competitive antagonist compounds that have activity at both mGluR2 and mGluR3 receptors. Compounds that bind to allosteric sites of the mGluR2 receptor may allow differentiation from the activities of these orthosteric ligands. Pharmacological manipulations of mGluR2 have been suggested to be useful for a variety of disorders (Marek, Current Opinion in Pharmacology, 2004, 4, 18-22). These include anxiety and related disorders (Tizzano et al., Pharmacol. Biochem., Behav., 2002, 73, 367-374), stress disorders (Eur J. Pharmacol., 2002, 435, 161-170), depression (Feinberg et al., Pharmacol Biochem, Behav., 2002, 73, 467-474), schizophrenia (Klodzinska et al., Pharmacol Biochem, Behav., 2002, 73, 327-332; Moghaddam and Adams, Science, 1998, 281, 1349-1352), pain disorders including chronic pain syndromes (Varney and Gereau, Curr. Drug Target CNS Neurol. Disorders, 2002, 1, 283-296), seizure disorders and epilepsy (Moldrich et al., Neuropharmacol., 2001, 41, 8-18), Parkinson's (Bradley et al., J. Neurosci., 2000, 20, 3085-3094), neurodegenerative disorders and brain injury (Bond et al., J. Pharmacol Exp. Ther., 2000, 294, 800-809; Allen et al., J. Pharmacol Exp. Ther., 1999, 290, 112-290), and substance abuse (Helton et al., Neuropharmacol., 1998, 36, 1511-1516).

Pin et al., European J. Pharmacology 375 (1999), pp. 277-294, describes the role of mGluR2 agonists and antagonists in regulating the activity of many synapses in the central nervous system, thereby affecting a wide number of physiological and pathological processes.

Johnson et al., J. Med. Chem. 2003, 46, 31893192, describes mGluR2 potentiators that have antianxiolytic activity.

All journal articles cited hereinabove are incorporated by reference herein in their entirety.

WO 01/56990 states that mGluR2 receptor potentiators may be effective in the treatment of neurological and psychiatric disorders associated with glutamate dysfunction, including: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder.

A need still exists for new drug therapies for the treatment of subjects suffering from or susceptible to the above disorders or conditions. In particular, a need still exists for new drugs having one or more improved properties (such as safety profile, efficacy, or physical properties) relative to those currently available.

### SUMMARY OF THE INVENTION

The invention is directed to a class of compounds, including the pharmaceutically acceptable salts of the compounds, having the structure of formula I:

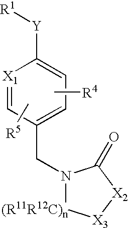

wherein Y is a bond, NR²², or O;

wherein, when Y is NR²² or O,

R¹ is alkyl, aryl, heteroaryl, heterocycloalkyl, or cycloalkyl each of which is optionally substituted with one, two, three or four R⁴¹, wherein each R⁴¹ is independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰²—NR¹⁰¹R¹⁰², NR¹⁰¹C(O)R¹⁰³, and —NR¹⁰¹S(O)₂R¹⁰³ wherein each of the R⁴¹ alkyl, heterocycloalkyl, cycloalkyl, aryl or heteroaryl is optionally substituted with one or more substituents independently selected from the group consisting of halogen, cyano, —R¹⁰¹, —OR¹⁰¹, —NR¹⁰¹R¹⁰², S(O)_{q}R¹⁰³, —S(O)₂NR¹⁰¹R¹⁰², —NR¹⁰¹S(O)₂R¹⁰³, —OC(O)R¹⁰³, C(O)OR¹⁰³, —C(O)NR¹⁰¹R¹⁰², NR¹⁰¹C(O)R¹⁰³, and C(O)R¹⁰³;

or, when R¹ is aryl, heteroaryl, cycloalkyl or heterocycloalkyl, two R⁴¹ substituents bonded to adjacent carbon atoms of R¹, together with the adjacent carbon atoms, form a heterocyclic or carbocyclic ring which is optionally substituted with one or more R¹⁰;

wherein each R¹⁰ is independently selected from the group consisting of hydrogen, —CN, halogen, —C(O)R¹⁰¹, —C(O)NR¹⁰¹R¹⁰², —NR¹⁰¹R¹⁰², —OR¹⁰¹, or —R¹⁰¹;

and when Y is a bond,

R¹ is either

(a) aryl, heteroaryl, heterocycloalkyl, or cycloalkyl wherein R¹ is optionally substituted with one, two, three or four R⁴¹, wherein each R⁴¹ is independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴¹ alkyl, heterocycloalkyl, cycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², NR¹⁰¹C(O)R¹⁰³ and C(O)R¹⁰³;

or wherein, when R¹ is aryl, heteroaryl, cycloalkyl or heterocycloalkyl, two R⁴¹ substituents bonded to adjacent carbon atoms of R¹, together with the adjacent carbon atoms, form a heterocyclic or carbocyclic ring which is optionally substituted with one or more R¹⁰;

or

(b) alkyl or alkenyl substituted with one, two, three or four R⁴² and further optionally substituted with halogen, wherein each R⁴² is independently selected from the group consisting of cyano, —OR¹⁰¹, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴² heterocycloalkyl, cycloalkyl, cycloalkenyl, aryl or heteroaryl is optionally substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², NR¹⁰¹C(O)R¹⁰³, and C(O)R¹⁰³;

X₁ is CR⁶ or N;

n is 1 or 2;

X₂ is O or CR⁷R⁸;

X₃ is NR²³, O, or CR²R³;

with the proviso that if X₂ is O, X₃ is CR²R³, and

with the proviso that if X₂ is CR⁷R⁸, X³ is NR²³ or O;

wherein

each of R² and R³ is independently selected from the group consisting of hydrogen, alkyl, aryl, heteroaryl, heterocycloalkyl, and cycloalkyl wherein the R² or R³ alkyl, aryl, heteroaryl, heterocycloalkyl, or cycloalkyl is optionally substituted with one, two, three or four R⁴³, wherein each R⁴³ is independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴³ alkyl, heterocycloalkyl, cycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —(O)OR¹⁰³, —C(O)NR¹⁰¹R¹⁰², NR¹⁰¹C(O)R¹⁰³, and C(O)R¹⁰³;

q is 0, 1 or 2;

or R² and R³ taken together with the carbon that R² and R³ are attached to form a carbocyclic or heterocyclic ring, optionally substituted with one, two, three or four R⁴³;

each R¹⁰¹ and each R¹⁰² is independently selected from the group consisting of hydrogen, alkyl, alkenyl, alkynyl, cycloalkyl, aryl, heterocycloalkyl and heteroaryl;

wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, hydroxyalkyl, carboxy, alkoxy, aryloxy, alkoxycarbonyl, aminocarbonyl, alkylaminocarbonyl and dialkylaminocarbonyl;

R¹⁰³ is independently selected from the group consisting of alkyl, alkenyl, cycloalkyl, aryl, heterocycloalkyl and heteroaryl and is optionally substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, hydroxyalkyl, alkoxy, and aryloxy;

R²² is hydrogen, alkyl, heterocycloalkyl, or cycloalkyl wherein the R²² alkyl, heterocycloalkyl, or cycloalkyl is optionally substituted with one, two, three or four alkyl, heterocycloalkyl, cycloalkyl, aryl, heteroaryl, halogen, or OR¹⁰¹, wherein the heterocycloalkyl, cycloalkyl, aryl, or heteroaryl substituent on R²² is optionally substituted with alkyl, cycloalkyl, halogen or OR¹⁰¹;

R²³ is alkyl, heterocycloalkyl, aryl, heteroaryl, or cycloalkyl wherein R²³ is optionally substituted with one, two, three or four alkyl, heterocycloalkyl, cycloalkyl, aryl, heteroaryl, halogen, or OR¹⁰¹, wherein the heterocycloalkyl, cycloalkyl, aryl, or heteroaryl substituent on R²³ is optionally substituted with alkyl, cycloalkyl, halogen or OR¹⁰¹;

each R⁷, R⁸, R¹¹ or R¹² is independently hydrogen, alkyl, aryl, heteroaryl, heterocycloalkyl, or cycloalkyl, wherein the R⁷, R⁸, R¹¹ or R¹² alkyl, aryl, heteroaryl, heterocycloalkyl, or cycloalkyl is optionally substituted with one, two, three or four groups independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³;

or when n is 2, the two R¹¹ groups together with the carbon atoms interconnecting them form a 5-7 membered carbocyclic or heterocylic ring that is optionally substituted with one or two groups independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³;

R⁴, R⁵ and R⁶ are each independently selected from the group consisting of hydrogen, halogen, alkyl optionally substituted with one or more halogens, alkoxy optionally substituted with one or more halogens, and cyano;

or if X₂ is O and X₃ is CR²R³, and two of the substituents R⁴, R⁵ and R⁶ are bonded to adjacent carbon atoms, the two of the substituents R⁴, R⁵ and R⁶ together with the adjacent carbon atoms form a heterocyclic or carbocyclic ring which is optionally substituted with one or more R¹⁰;

or, if X₂ is CR⁷R⁸ and X₃ is NR²³, and two of the substituents R⁴, R⁵ and R⁶ are bonded to adjacent carbon atoms, the two of the substituents R⁴, R⁵ and R⁶ together with the adjacent carbon atoms form a carbocyclic or heterocyclic ring which is optionally substituted with one or more R¹⁰;

or R⁶ and R¹ taken together with the atoms that R⁶ and R¹ are attached to form a carbocyclic or heterocyclic ring that is optionally substituted with alkyl, cycloalkyl, halogen, or OR¹⁰¹;

or R⁶ and R⁴¹ taken together with the atoms that R⁶ and R⁴¹ are attached to form a carbocylic or heterocylic ring that is optionally substituted with alkyl, cycloalkyl, halogen, or OR¹⁰¹.

In one embodiment of the invention, n=1.

In another embodiment of the invention, n=2.

In another embodiment of the compound of formula I, X₃ is CR²R³ wherein one or both of R² and R³ are alkyl.

In another embodiment of the compound of formula I, X₃ is CR²R³ wherein one of R² and R³ is hydrogen and the other of R² and R³ is alkyl or aryl.

In another embodiment of the invention, X₁ is N.

In another embodiment of the compound of formula I, Y is a bond and R¹ is phenyl, optionally substituted as in the compound of formula I.

In another embodiment of the compound of formula I, R¹ is pyridyl or pyrimidyl, optionally substituted as in the compound of formula I. In an example of this embodiment, R¹ is pyridyl substituted with dialkylamino or with pyrrolidinyl or with morpholinyl, wherein the dialkylamino or pyrrolidinyl or morpholinyl group is preferably ortho to the pyridyl ring nitrogen. The R¹ pyridyl may be optionally fused to a benzene ring. In one example of this embodiment, X₁ is N. In another example of this embodiment, X₁ is CR⁶.

When R¹ is pyridyl, and Y in formula I is NR²² or O, the nitrogen of the pyridyl ring may be ortho to the bond connecting the pyridyl ring to Y, meta to the bond connecting the pyridyl ring to Y, or para to the bond connecting the pyridyl ring to Y. Preferably, the nitrogen of the pyridyl ring is ortho to the bond connecting the pyridyl ring to Y. When R¹ is pyridyl, and Y is a bond, the nitrogen of the pyridyl ring may be ortho to the bond connecting the pyridyl ring to the ring containing X₁ in formula I, meta to the bond connecting the pyridyl ring to the ring containing X₁, or para to the bond connecting the pyridyl ring to the ring containing X₁. Preferably, the nitrogen of the pyridyl ring is ortho to the bond connecting the pyridyl ring to the ring containing X₁.

When R¹ is pyrimidinyl, and Y in formula I is NR²² or O, the two nitrogens of the pyrimidinyl ring may be each ortho to the bond connecting the pyrimidinyl ring to Y, each meta to the bond connecting the pyrimidinyl ring to Y, or ortho and para, respectively, to the bond connecting the pyrimidinyl ring to Y. Preferably, the two nitrogens of the pyrimidinyl ring are ortho and para, respectively, to the bond connecting the pyrimidinyl ring to Y. When R¹ is pyrimidinyl, and Y in formula I is a bond, the two nitrogens of the pyrimidinyl ring may be each ortho to the bond connecting the pyrimidinyl ring to the ring containing X₁ in formula I, each meta to the bond connecting the pyrimidinyl ring to the ring containing X₁, or ortho and para, respectively, to the bond connecting the pyrimidinyl ring to the ring containing X₁. Preferably, the two nitrogens of the pyrimidinyl ring are ortho and para, respectively, to the bond connecting the pyrimidinyl ring to the ring containing X₁.

In another embodiment of the invention, the R¹ heterocycloalkyl contains a nitrogen that is directly bonded to Y, wherein the R¹ heterocycloalkyl is optionally substituted as defined in formula I. In an example of this embodiment of the invention, R¹ is pyrrolidinyl optionally fused to a benzene ring that is optionally substituted with halogen.

In another embodiment of the invention, R¹ is aryl, cycloalkyl, heteroaryl, or heterocycloalkyl and is optionally substituted as in formula I.

In an example of this embodiment of the invention, R¹ is cyclobutyl, cyclopentyl optionally fused to a benzene ring, cyclohexyl optionally fused to a benzene ring, cycloheptyl, decalinyl, norbornyl, morpholinyl, or tetrahydropyranyl, optionally substituted as in the compound of formula I. In one example of this embodiment, X₁ is N. In another example of this embodiment, X₁ is CR⁶.

In another example of this embodiment of the invention, R¹ is phenyl which may be substituted by one or two substituents R⁴¹ as defined in formula I. If there are two substituents R⁴¹, the two substituents R⁴¹ may be, for example, ortho and para relative to the R¹—Y bond, or both meta relative of the R¹—Y bond. As an example, the one or two substituents R⁴¹ may be independently selected from the group consisting of halogen, cyano, alkyl optionally substituted with halogen, alkoxy optionally substituted with halogen, carboxyalkyl, alkylcarbonyl, and cycloalkoxy optionally substituted with alkyl or halogen. The R¹ phenyl may be optionally fused to a heterocylic or carbocyclic ring to form a 2,3-dihydro-1-benzofuranyl, chromanyl, 2,3-dihydro-1,4-benzodioxinyl, N-alkylindolinyl, or quinolinyl group. A substituent on the R¹ phenyl ring, taken together with an R⁶ substituent on the central phenyl ring in formula I, may form a 5- or 6-membered carbocyclic ring. Preferably, where R¹ is phenyl substituted as described in this paragraph, —Y— is a bond. In one example of this embodiment, X₁ is N. In another example of this embodiment, X₁ is CR⁶.

In another example of this embodiment of the invention, R¹ is furanyl, benzofuranyl, thiazolyl or pyrrolyl optionally substituted with one or two alkyl.

In another embodiment of the invention, R¹ is alkyl substituted with one, two, three or four R⁴², wherein each R⁴² is independently selected from the group consisting of —OR¹⁰¹, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴² alkyl, heterocycloalkyl, cycloalkyl, aryl or heteroaryl is optionally independently substituted as in formula I.

In another embodiment of the invention, Y is O and R¹ is alkyl such as methyl, ethyl, propyl, or butyl where R¹ is substituted with amino, alkylamino, dialkylamino, cyclopropyl, cyclobutyl, cyclopentyl, cyclohexyl, tetrahydropyranyl, pyridyl, phenyl optionally substituted with one or two groups which are independently alkoxy or halogen or alkyl or cyano or pyrazolyl, benzimidazolyl optionally substituted with alkyl, such as 2-benzimidazolyl optionally substituted with alkyl at the nitrogen in the 1 position of the benzimidazolyl, isoxazolyl optionally substituted with one or two groups which are independently alkyl or phenyl, pyrazolyl optionally substituted with one or two groups which are independently alkyl or phenyl, phenoxy optionally substituted with halogen. In one example of this embodiment, X₁ is N. In another example of this embodiment, X₁ is CR⁶.

In another embodiment of the invention, Y is O and or R⁶ and R¹ taken together with the atoms that R⁶ and R¹ are attached to form a tetrahydropyran or tetrahydrofuran optionally substituted with alkyl.

In another embodiment of the invention, the R⁴¹ or R⁴² heterocycloalkyl contains a nitrogen that is directly bonded to R¹ and the R⁴¹ or R⁴² heterocycloalkyl is optionally substituted as in formula I.

In another embodiment of the invention, when only one of R² and R³ is hydrogen, the carbon to which R² and R³ are bonded is an (R) chiral center. In another embodiment of the invention, when only one of R² and R³ is hydrogen, the carbon to which R² and R³ are bonded is an (S) chiral center.

In another embodiment of the invention, when n is 2 and R¹¹ and R¹² groups taken together with the two carbon atoms interconnecting them form a 5-7 membered carbocyclic or heterocyclic ring that is optionally substituted as in formula I, the 5-7 membered carbocyclic or heterocyclic ring is cis-fused to the ring containing X₃ and X₂.

In another embodiment of the invention, when X₃ is NR²³ or O, n is 1.

In another embodiment of the invention, when X₃ is NR²³ or O, n is 2.

In another embodiment of the invention, X₂ is O.

In another embodiment of the invention X₂ is CH₂ and X₃ is NR²³.

In another embodiment of the invention, when Y is O, R¹ is alkyl substituted with aryl, heteroaryl, cycloalkyl or heterocycloalkyl, such that R¹ has a chiral center. The chiral center may be at the point of substitution or may be at a tertiary carbon in the alkyl chain. In an example of such an embodiment, R¹ is alkyl substituted with cyclohexyl or norbornyl or phenyl optionally substituted with alkyl, to form a chiral center at the point of substitution. In another example of such an embodiment, R¹ is 2-propyl wherein one of the methyl groups of the propyl is substituted with phenyl or cyclohexyl.

In another embodiment of the invention, when X₃═O, each of R¹¹ and R¹² is independently hydrogen, aryl, heteroaryl, cycloalkyl or heterocycloalkyl, optionally substituted as formula I.

In another embodiment of the invention, R²³ is alkyl or cycloalkyl optionally substituted with one or two alkyl.

In another embodiment of the invention, in the compound of formula I, the group

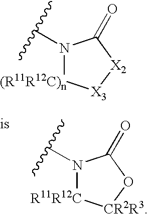

In another embodiment of the invention, in the compound of formula I, the group

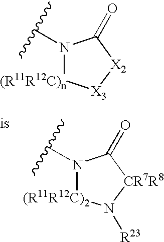

In another embodiment of the invention, the compound of formula I has the following formula, with the absolute stereochemistry as shown:

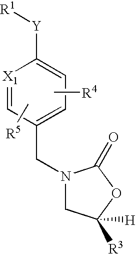

In an example of this embodiment, R³ is alkyl optionally substituted as in Formula I, preferably methyl optionally substituted as in Formula I.

In another example of this embodiment, R¹ is phenyl optionally substituted with one, two, three or four R⁴¹, wherein each R⁴¹ is independently selected from the group consisting of halogen, —CN, —OR¹⁰¹, alkyl, cycloalkyl, heterocycloalkyl, aryl, heteroaryl, —C(O)R¹⁰¹, —C(O)OR¹⁰¹, and —NR¹⁰¹R¹⁰², wherein each of the R⁴¹ alkyl, heterocycloalkyl, cycloalkyl, aryl or heteroaryl is optionally independently substituted as in Formula I; or wherein two R⁴¹ substituents bonded to adjacent carbon atoms of R¹, together with the adjacent carbon atoms, form a heterocyclic or carbocyclic ring which is optionally substituted with one or more R¹⁰, wherein each R¹⁰ is defined as in Formula I; or wherein R⁶ and R⁴¹ taken together with the atoms that R⁶ and R⁴¹ are attached to form a carbocylic or heterocyclic ring that is optionally substituted with alkyl, cycloalkyl, halogen, or OR¹⁰¹.

In another example of this embodiment, Y is NR²² or O and R¹ is alkyl optionally substituted with one, two, three or four R⁴¹, wherein each R⁴¹ is independently selected from the group consisting of alkyl, cycloalkyl, heterocycloalkyl, aryl, and heteroaryl and each R⁴¹ is optionally independently substituted as in Formula I.

In another example of this embodiment, Y is a bond and R¹ is alkyl substituted with one, two, three or four R⁴², wherein each R⁴² is independently selected from the group consisting of alkyl, cycloalkyl, heterocycloalkyl, aryl, and heteroaryl and each R⁴² is optionally independently substituted as in Formula I.

In another embodiment of the invention, the compound of formula I has the following formula, with the absolute stereochemistry as shown:

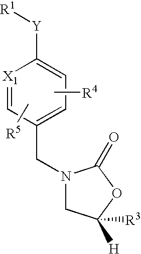

In an example of this embodiment, R³ is alkyl optionally substituted as in Formula I, preferably methyl optionally substituted as in Formula I.

In another example of this embodiment, R¹ is phenyl optionally substituted with one, two, three or four R⁴¹, wherein each R⁴¹ is independently selected from the group consisting of halogen, —CN, —OR¹⁰¹, alkyl, cycloalkyl, heterocycloalkyl, aryl, heteroaryl, —C(O)R¹⁰¹, —C(O)OR¹⁰¹, and —NR¹⁰¹R¹⁰², wherein each of the R⁴¹ alkyl, heterocycloalkyl, cycloalkyl, aryl or heteroaryl is optionally independently substituted as in Formula I; or wherein two R⁴¹ substituents bonded to adjacent carbon atoms of R¹, together with the adjacent carbon atoms, form a heterocylic or carbocyclic ring which is optionally substituted with one or more R¹⁰, wherein each R¹⁰ is defined as in Formula I; or wherein or R⁶ and R⁴¹ taken together with the atoms that R⁶ and R⁴¹ are attached to form a carbocyclic or heterocyclic ring that is optionally substituted with alkyl, cycloalkyl, halogen, or OR¹⁰¹.

In another example of this embodiment, Y is NR²² or O and R¹ is alkyl optionally substituted with one, two, three or four R⁴¹, wherein each R⁴¹ is independently selected from the group consisting of alkyl, cycloalkyl, heterocycloalkyl, aryl, and heteroaryl and each R⁴¹ is optionally independently substituted as in Formula I.

In another example of this embodiment, Y is a bond and R¹ is alkyl substituted with one, two, three or four R⁴², wherein each R⁴² is independently selected from the group consisting of alkyl, cycloalkyl, heterocycloalkyl, aryl, and heteroaryl and each R⁴² is optionally independently substituted as in Formula I.

Exemplary compounds according to the invention include the compounds disclosed in Table 1 herein or pharmaceutically acceptable salts thereof.

The compounds of formula I are useful for the treatment of a variety of neurological and psychiatric disorders associated with glutamate dysfunction, including: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder. Accordingly, in one embodiment, the invention provides a method for treating a condition in a mammal, such as a human, selected from the conditions above, comprising administering a compound of formula I to the mammal. The mammal is preferably a mammal in need of such treatment. As an example, the invention provides a method for treating a condition selected from migraine, anxiety disorders, schizophrenia, and epilepsy. Exemplary anxiety disorders are generalized anxiety disorder, social anxiety disorder, panic disorder, post-traumatic stress disorder and obsessive-compulsive disorder.

In another embodiment the present invention provides methods of treating neurological and psychiatric disorders associated with glutamate dysfunction, comprising: administering to a patient in need thereof an amount of a compound of formula I effective in treating such disorders. The compound of formula I is optionally used in combination with another active agent. Such an active agent may be, for example, a metabotropic glutamate receptor agonist.

The invention is also directed to a pharmaceutical composition comprising a compound of formula I, and a pharmaceutically acceptable carrier. The composition may be, for example, a composition for treating a condition selected from the group consisting of acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder, wherein the composition contains an amount of the compound of formula I that is effective in the treatment of such conditions. The composition may be, as another example, a composition comprising an mGluR-2 potentiating amount of the compound of formula I.

The composition may also further comprise another active agent. Such an active agent may be, for example, a metabotropic glutamate receptor agonist.

### DETAILED DESCRIPTION OF THE INVENTION

This detailed description of embodiments is intended only to acquaint others skilled in the art with Applicants' invention, its principles, and its practical application so that others skilled in the art may adapt and apply the invention in its numerous forms, as it may be best suited to the requirements of a particular use. This invention, therefore, is not limited to the embodiments described in this specification, and may be variously modified.

### Abbreviations and Definitions

TABLE A
Abbreviations

| | |
|---|---|
| 1-HOAT | 1-hydroxy-7-azabenzotriazole |
| 1-HOBt | 1-hydroxybenzotriazole hydrate |
| ADP | Adenosine diphosphate (the natural ligand of P2Y12) |
| AMP | Adenosine monophospate |
| ASA | Acetylsalicylic acid |
| ATP | Adenosine triphosphate |
| Bn | Benzyl group |
| Boc | tert-butoxycarbonyl |
| BOP-Cl | bis(2-oxo-3-oxazolidinyl)phosphinic chloride |
| br | Broad |
| BSA | Bovine serum albumin |
| Cbz | benzyloxycarbonyl |
| CD₃OD | Deuterated methanol |
| CDCl₃ | Deuterated chloroform |
| CDI | 1,1′-carbonyldiimidazole |
| d | Doublet |
| DBN | 1,5-diazabicyclo[4.3.0]non-5-ene |
| DBU | 1,8-diazabicyclo[5.4.0]undec-7-ene |
| DCC | 1,3-dicyclohexylcarbodiimide |
| DCM | dichloromethane |
| DMC | 2-chloro-1,3-dimethylimidazolinium chloride |
| dd | Doublet of doublets |
| DEPC | diethyl cyanophosphonate |
| DIEA | diisopropylethylamine |
| DMF | N,N-dimethylformamide |
| DMSO | dimethyl sulphoxide |
| DPBS | Dulbecco's Phosphate Buffered Saline |
| EBSS | Earle's Balanced Salt Solution |
| EDC | 1-(3-dimethylaminopropyl)-3-ethylcarbodiimide |
| | hydrochloride |
| EDTA | ethylenediaminetetraacetic acid |
| EGTA | ethyleneglycol-bis(β-aminoethyl)-N,N,N′,N′-tetraacetic |
| | Acid |
| ESI | Electrospray Ionization for mass spectrometry |
| Et₃N | triethylamine |
| EtOAc | ethyl acetate |
| EtOH | ethanol |
| FBS | Fetal bovine serum |
| Fmoc | Fluorene methyloxycarbonyl |
| HATU | O-(7-azabenzotriazol-1-yl)-N,N,N′,N′- |
| | tetramethyluronium hexafluorophosphate |
| HBTU | O-benzotriazol-1-yl-N,N,N′,N′-tetramethyluronium |
| | hexafluorophosphate |
| HCl | Hydrochloric acid |
| HEK | Human embryonic kidney |
| HEPES | 4-(2-hydroxyethyl)-1-Piperazineethane sulfonic acid |
| HOBT | 1-hydroxybenzotriazole |
| HRMS | High Resolution Mass Spectroscopy (electrospray |
| | ionization positive scan) |
| K₃PO₄ | Potassium phosphate |
| LCMS | Liquid Chromatography - Mass Spectroscopy |
| LRMS | Low Resolution Mass Spectroscopy (electrospray or |
| | thermospray ionization positive scan) |
| LRMS | Low Resolution Mass Spectroscopy (electrospray |
| (ES⁻) | ionization negative scan) |
| m | Multiplet |
| m/z | Mass spectrum peak |
| MEM | Minimum essential medium |
| MeOH | methanol |
| MHz | Megahertz |
| MS | Mass spectroscopy |
| NaH | Sodium hydride |
| NMM | N-methylmorpholine |
| NMP | 1-methyl-2-pyrrolidinone |
| NMR | Nuclear Magnetic Resonance |
| PG | Protecting group. Exemplary protecting groups include |
| | Boc, Cbz, Fmoc and benzyl |
| Pg. | Page |
| PPP | Platelet poor plasma |
| PRP | Platelet rich plasma |
| q | Quartet |
| Rpm | Revolutions per minute |
| s | Singlet |
| t | Triplet |
| TFA | trifluoroacetic acid |
| THF | Tetrahydrofuran |
| TLC | Thin layer chromatography |
| Vol. | Volume |
| δ | Chemical shift |

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, iso-amyl, hexyl and the like.

The term “alkenyl” refers to a linear or branched-chain hydrocarbyl substituent containing one or more double bonds and from two to twenty carbon atoms; in another embodiment, from two to twelve carbon atoms; in another embodiment, from two to six carbon atoms; and in another embodiment, from two to four carbon atoms. Examples of alkenyl include ethenyl (also known as vinyl), allyl, propenyl (including 1-propenyl and 2-propenyl) and butenyl (including 1-butenyl, 2-butenyl and 3-butenyl). The term “alkenyl” embraces substituents having “cis” and “trans” orientations, or alternatively, “E” and “Z” orientations.

The term “benzyl” refers to methyl radical substituted with phenyl, i.e., the following structure:

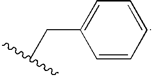

The term “carbocyclic ring” refers to a saturated cyclic, partially saturated cyclic, or aromatic ring containing from 3 to 14 carbon ring atoms (“ring atoms” are the atoms bound together to form the ring). A carbocyclic ring typically contains from 3 to 10 carbon ring atoms. Examples include cyclopropyl, cyclobutyl, cyclopentyl, cyclopentenyl, cyclopentadienyl, cyclohexyl, cyclohexenyl, cyclohexadienyl, and phenyl. A “carbocyclic ring system” alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluorenyl, and decalinyl.

The term “heterocyclic ring” refers to a saturated cyclic, partially saturated cyclic, or aromatic ring containing from 3 to 14 ring atoms (“ring atoms” are the atoms bound together to form the ring), in which at least one of the ring atoms is a heteroatom that is oxygen, nitrogen, or sulfur, with the remaining ring atoms being independently selected from the group consisting of carbon, oxygen, nitrogen, and sulfur.

The term “cycloalkyl” refers to a saturated carbocyclic substituent having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substitutents, unless otherwise specified, are each bound to a carbon atom of the cycloalkyl group. The fused C₆-C₁₀ aromatic ring or to a 5-10-membered heteroaromatic ring may be optionally substituted with halogen, C₁-C₆ alkyl, C₃-C₁₀ cycloalkyl, or ═O.

The term “cycloalkenyl” refers to a partially unsaturated carbocyclic substituent having three to fourteen carbon atoms, typically three to ten carbon atoms. Examples of cycloalkenyl include cyclobutenyl, cyclopentenyl, and cyclohexenyl.

A cycloalkyl or cycloalkenyl may be a single ring, which typically contains from 3 to 6 ring atoms. Examples include cyclopropyl, cyclobutyl, cyclopentyl, cyclopentenyl, cyclopentadienyl, cyclohexyl, cyclohexenyl, cyclohexadienyl, and phenyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄-C₁₀ carbocyclic ring, such as a C₅ or a C₆ carbocylic ring, or to a 4-10-membered heterocyclic ring, wherein a group having such a fused aryl group as a substituent is bound to an aromatic carbon of the aryl group. When such a fused aryl group is substituted with one more substituents, the one or more substitutents, unless otherwise specified, are each bound to an aromatic carbon of the fused aryl group. The fused C₄-C₁₀ carbocyclic or 4-10-membered heterocylic ring may be optionally substituted with halogen, C₁-C₆ alkyl, C₃-C₁₀ cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of carbon atoms in a hydrocarbyl substituent (e.g., alkyl, alkenyl, cycloalkyl, cycloalkenyl, aryl, etc.) is indicated by the prefix “Cₓ—C_{y}-,” wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl refers to saturated cycloalkyl containing from 3 to 6 carbon ring atoms.

In some instances, the number of atoms in a cyclic substituent containing one or more heteroatoms (e.g., heteroaryl or heterocycloalkyl) is indicated by the prefix “X—Y-membered”, wherein x is the minimum and y is the maximum number of atoms forming the cyclic moiety of the substituent. Thus, for example, 5-8-membered heterocycloalkyl refers to a heterocycloalkyl containing from 5 to 8 atoms, including one or more heteroatoms, in the cyclic moiety of the heterocycloalkyl.

The term “hydrogen” refers to hydrogen substituent, and may be depicted as —H.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “nitro” means —NO₂.

The term “cyano” (also referred to as “nitrile”) means —CN, which also may be depicted:

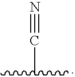

The term “carbonyl” means —C(O)—, which also may be depicted as:

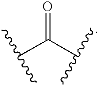

The term “amino” refers to —NH₂.

The term “alkylamino” refers to an amino group, wherein at least one alkyl chain is bonded to the amino nitrogen in place of a hydrogen atom. Examples of alkylamino substituents include monoalkylamino such as methylamino (exemplified by the formula —NH(CH₃)), which may also be depicted:

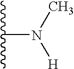

and dialkylamino such as dimethylamino, (exemplified by the formula —N(CH₃)₂, which may also be depicted:

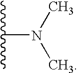

The term “aminocarbonyl” means —C(O)—NH₂, which also may be depicted as:

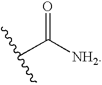

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is a fluorine.

The prefix “halo” indicates that the substituent to which the prefix is attached is substituted with one or more independently selected halogen substituents. For example, haloalkyl refers to an alkyl that is substituted with at least one halogen substituent. Where more than one hydrogen is replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The term “oxo” refers to ═O.

The term “oxy” refers to an ether substituent, and may be depicted as —O—.

The term “alkoxy” refers to an alkyl linked to an oxygen, which may also be represented as

—O—R, wherein the R represents the alkyl group. Examples of alkoxy include methoxy, ethoxy, propoxy and butoxy.

The term “alkoxycarbonyl” means —C(O)—O-alkyl. For example, “ethoxycarbonyl” may be depicted as:

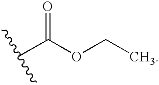

Examples of other alkoxycarbonyl include methoxycarbonyl, ethoxycarbonyl, propoxycarbonyl, butoxycarbonyl, pentoxycarbonyl, and hexyloxycarbonyl. In another embodiment, where the carbon atom of the carbonyl is attached to a carbon atom of a second alkyl, the resulting functional group is an ester.

The terms “thio” and “thia” mean a divalent sulfur atom and such a substituent may be depicted as —S—. For example, a thioether is represented as “alkyl-thio-alkyl” or, alternatively, alkyl-S-alkyl.

The term “thiol” refers to a sulfhydryl substituent, and may be depicted as —SH.

The term “sulfonyl” refers to —S(O)₂—, which also may be depicted as:

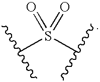

Thus, for example, “alkyl-sulfonyl-alkyl” refers to alkyl-S(O)₂-alkyl. Examples of alkylsulfonyl include methylsulfonyl, ethylsulfonyl, and propylsulfonyl.

The term “aminosulfonyl” means —S(O)₂—NH₂, which also may be depicted as:

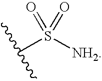

The term “heterocycloalkyl” refers to a saturated or partially saturated ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocycloalkyl alternatively may comprise 2 or 3 rings fused together, wherein at least one such ring contains a heteroatom as a ring atom (e.g., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆-C₁₀ aromatic ring or to a 5-10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one more substituents, the one or more substitutents, unless otherwise specified, are each bound to a heteroatom of the heterocycloalkyl group or to a carbon atom of the heterocycloalkyl group. The fused C₆-C₁₀ aromatic ring or 5-10-membered heteroaromatic ring may be optionally substituted with halogen, C₁-C₆ alkyl, C₃-C₁₀ cycloalkyl, C₁-C₆ alkoxy, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls include furanyl, dihydrofuranyl, tetrahydrofuranyl, thiophenyl (also known as “thiofuranyl”), dihydrothiophenyl, tetrahydrothiophenyl, pyrrolyl, isopyrroyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiadiazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or o-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryls include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substitutents, unless otherwise specified, are each bound to an aromatic carbon of the heteroaryl group or to a heteroatom of the heteroaryl group. The fused C₄-C₁₀ carbocylic or 4-10-membered heterocyclic ring may be optionally substituted with halogen, C₁-C₆ alkyl, C₃-C₁₀ cycloalkyl, or ═O.

The term “ethylene” refers to the group —CH₂—CH₂—.

The term “propylene” refers to the group —CH₂—CH₂—CH₂—.

A substituent is “substitutable” if it comprises at least one carbon, sulfur, oxygen or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated. In one embodiment, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁-C₆-prefix on C₁-C₆-alkylcycloalkyl means that the alkyl moiety of the alkylcycloalkyl contains from 1 to 6 carbon atoms; the C₁-C₆ prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution may only occur on the alkyl moiety, the substituent would be described as “alkoxyhaloalkyl.” If the halogen substitution may occur on both the alkyl moiety and the alkoxy moeity, the substituent would be described as “haloalkoxyhaloalkyl.”

When a substituent is comprised of multiple moieties, unless otherwise indicated, it is the intention for the final moiety to serve as the point of attachment to the remainder of the molecule. For example, in a substituent A-B-C, moiety C is attached to the remainder of the molecule. In a substituent A-B-C-D, moiety D is attached to the remainder of the molecule. Similarly, in a substituent aminocarbonylmethyl, the methyl moiety is attached to the remainder of the molecule, where the substituent may also be be depicted as

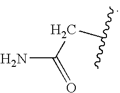

In a substituent trifluoromethylaminocarbonyl, the carbonyl moiety is attached to the remainder of the molecule, where the substituent may also be depicted as

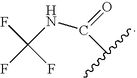

If substituents are described as being “independently selected” from a group, each substituent is selected independent of the other. Each substituent therefore may be identical to or different from the other substituent(s).

### Isomers

When an asymmetric center is present in a compound of formulae I, hereinafter referred to as the compound of the invention, the compound may exist in the form of optical isomers (enantiomers). In one embodiment, the present invention comprises enantiomers and mixtures, including racemic mixtures of the compounds of formulae I. In another embodiment, for compounds of formulae I that contain more than one asymmetric center, the present invention comprises diastereomeric forms (individual diastereomers and mixtures thereof) of compounds. When a compound of formulae I contains an alkenyl group or moiety, geometric isomers may arise.

### Tautomeric Forms

The present invention comprises the tautomeric forms of compounds of formulae I. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

### Salts

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formulae I-V with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glucoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

In one embodiment, hemisalts of acids and bases may also be formed, for example, hemisulphate and hemicalcium salts.

The compounds of the invention may exist in both unsolvated and solvated forms.

### Prodrugs

Also within the scope of the present invention are so-called “prodrugs” of the compound of the invention. Thus, certain derivatives of the compound of the invention which may have little or no pharmacological activity themselves can, when administered into or onto the body, be converted into the compound of the invention having the desired activity, for example, by hydrolytic cleavage. Such derivatives are referred to as “prodrugs.” Further information on the use of prodrugs may be found in “Pro-drugs as Novel Delivery Systems, Vol. 14, ACS Symposium Series (T Higuchi and W Stella) and “Bioreversible Carriers in Drug Design,” Pergamon Press, 1987 (ed. E B Roche, American Pharmaceutical Association). Prodrugs in accordance with the invention can, for example, be produced by replacing appropriate functionalities present in the compounds of any of formulae I with certain moieties known to those skilled in the art as “pro-moieties” as described, for example, in “Design of Prodrugs” by H Bundgaard (Elsevier, 1985).

### Isotopes

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

### Administration and Dosing

Typically, a compound of the invention is administered in an amount effective to treat a condition as described herein. The compounds of the invention are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### Use in the Preparation of a Medicament

In another embodiment, the invention comprises the use of one or more compounds of the invention for the preparation of a medicament for the treatment of the conditions recited herein.

### Pharmaceutical Compositions

For the treatment of the conditions referred to above, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically-acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formulae I are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gellan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

### Co-Administration

The compounds of the present invention can be used, alone or in combination with other therapeutic agents, in the treatment of various conditions or disease states. The compound(s) of the present invention and other therapeutic agent(s) may be may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Kits

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### Intermediates

In another embodiment, the invention relates to the novel intermediates useful for preparing the compounds of the invention

### General Synthetic Schemes

The compounds of the formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of formula I, or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

Scheme I illustrates a method for the preparation of compounds of formula v, where R¹ to R⁵, X¹, Y and n are defined as above. Referring to scheme I, a compound of formula iv can be synthesized by treating an amine of formula iii with an aldehyde of formula ii in the presence of suitable reducing agents such as NaBH(OAc)₃ or Na(CN)BH₃ in solvents such as methylene chloride, dichloroethane, DMF or THF, at about room temperature. Other suitable conditions for this transformation include treatment of an amine of formula iii with an aldehyde of formula II in a solvent such as methanol or ethanol at room temperature, followed by treatment with a reducing agent such as NaBH₄ or NaCNBH₃, which also produce the desired compounds of formula iv. A compound of formula v can be synthesized from an aminoalcohol of formula iv by treating the aminoalcohol of formula iv with a suitable carbonyl reagent such as phosgene, triphosgene, or carbonyldiimidazole in a suitable solvent such as ether, THF or DMF at a temperature between 0° C. and 100° C. for a period between 1 h and 24 h. Preferred conditions for the synthesis of a compound of formula v from a compound of formula iv are carbonyldiimidazole in THF at a temperature between room temperature and 80° C. for about 3 h.

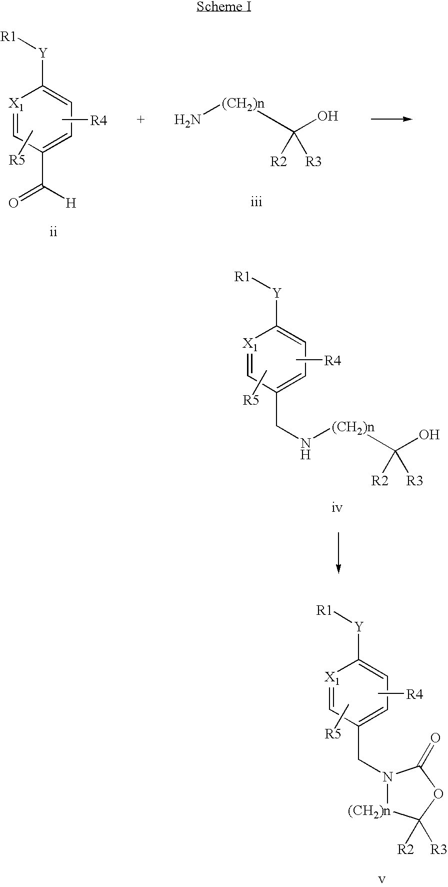

Scheme II illustrates a method for the preparation of compounds of formula i, where R¹ to R⁵, X¹-X³, Y and n are defined as above, and X is a leaving group such as Cl, Br, I, triflate, mesylate or tosylate. Referring to scheme II, a compound of formula i can be prepared from the alkylation of a compound of formula vii with a compound of formula vi in the presence of a suitable base, such as, but not limited to, sodium hydride, sodium carbonate, potassium carbonate, potassium tert-butoxide or sodium ethoxide, in a solvent such THF, DMF or DMSO, at a temperature between 40° C. and 150° C. with or without microwave heating.

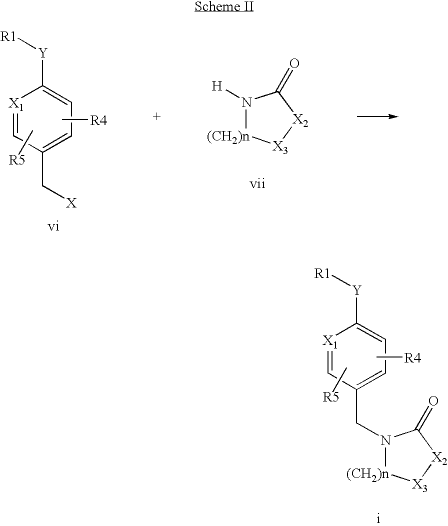

Scheme III illustrates a method for the preparation of compounds of formula ix, where R⁴, R⁵, and X¹ are defined as above, X is a leaving group such as Cl, Br, I, mesylate or tosylate, and Ar is an aryl or heteroaryl group. Referring to scheme III, a compound of formula ix can be prepared from the Suzuki coupling of a compound of formula viii with an aryl- or heteroarylboronic acid in the presence of a catalyst such as palladium (0) tetrakis(triphenylphosphine), palladium (II) acetate, allyl palladium chloride dimer, tris(dibenzylideneacetone)dipalladium (0), tris(dibenzylideneacetone)dipalladium (0) chloroform adduct, palladium (II) chloride or dichloro[1,1′-bis(diphenylphosphino)ferrocene]palladium (II) dichloromethane adduct, in the presence or absence of a base such as potassium phosphate, potassium acetate, sodium acetate, cesium acetate, sodium carbonate, lithium carbonate, potassium carbonate, cesium fluoride or cesium carbonate, preferably sodium carbonate. This reaction is typically carried out in an inert solvent such as dimethyl ethylene glycol ether (DME), 1,4-dioxane, acetonitrile, methyl sulfoxide, tetrahydrofuran, ethanol, methanol, 2-propanol, or toluene, in the presence or absence of about 1%-10% water, preferably about 5% water, with or without microwave assisted heating at a temperature from about 0° C. to about 200° C., preferably from about 60° C. to about 100° C.

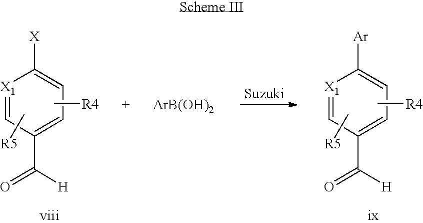

Scheme IV illustrates a method for the preparation of compounds of formula ix, wherein R¹-R⁵, X¹-X³ and n are defined as above, X is a leaving group such as Cl, Br, I, mesylate or tosylate, and Ar is an aryl or heteroaryl group. Referring to scheme IV, a compound of formula ix can be prepared from the Suzuki coupling of a compound of formula x with an aryl- or heteroarylboronic acid in the presence of a catalyst such as palladium (0) tetrakis(triphenylphosphine), palladium (II) acetate, allyl palladium chloride dimer, tris(dibenzylideneacetone)dipalladium (0), tris(dibenzylideneacetone)dipalladium (0) chloroform adduct, palladium (II) chloride or dichloro[1,1′-bis(diphenylphosphino)ferrocene]palladium (II) dichloromethane adduct, in the presence or absence of a base such as potassium phosphate, potassium acetate, sodium acetate, cesium acetate, sodium carbonate, lithium carbonate, potassium carbonate, cesium fluoride or cesium carbonate, preferably sodium carbonate. This reaction is typically carried out in an inert solvent such as dimethyl ethylene glycol ether (DME), 1,4-dioxane, acetonitrile, methyl sulfoxide, tetrahydrofuran, ethanol, methanol, 2-propanol, or toluene, in the presence or absence of about 1%-10% water, preferably about 5% water, with or without microwave assisted heating at a temperature from about 0° C. to about 200° C., preferably from about 60° C. to about 100° C.

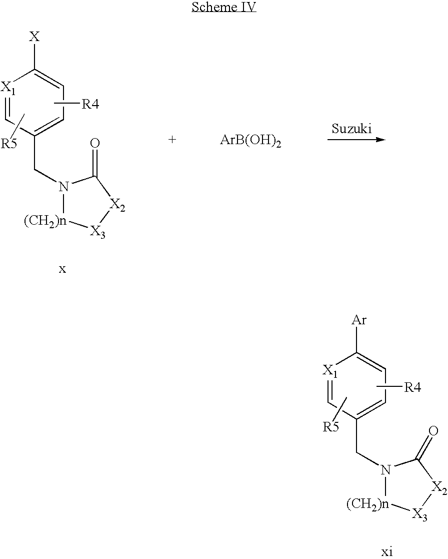

Scheme V illustrates a method for the preparation of compounds of formula xiii, where R¹ to R⁵, X¹-X³, Y and n are defined as above, and X is a leaving group such as Cl, Br, I, triflate, mesylate or tosylate. Referring to scheme V, a compound of formula xiii can be prepared from the alkylation of a compound of formula R₁OH or R₁R₂NH with a compound of formula xii in the presence of a suitable base, such as, but not limited to, triethylamine, diisopropylethylamine, sodium hydride, sodium carbonate, potassium carbonate, potassium tert-butoxide or sodium ethoxide, in a solvent such THF, DMF or DMSO, at a temperature between 40° C. and 150° C. with or without microwave heating.

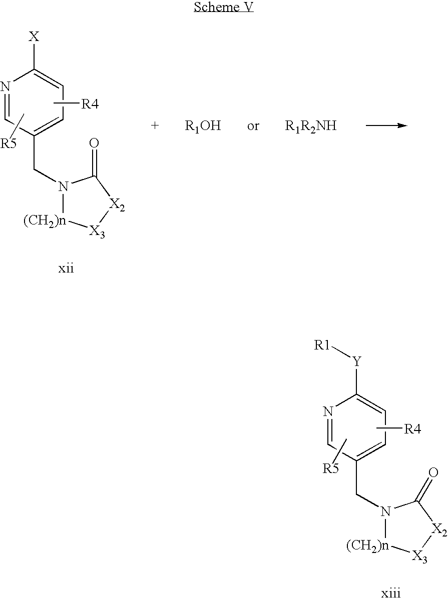

Scheme VI illustrates a method for the preparation of compounds of formula xv, where R⁴-R⁵ are defined as above, and R¹ is an optionally substituted alkyl or cycloalkyl group. Referring to scheme VI, a compound of formula xv can be prepared from the coupling of a compound of formula R₁OH with a compound of formula xiv in the presence of a suitable coupling reagent such as diethylazodicarboxylate (DEAD) or di-tert-butylazodicarboxylate and a phosphine, such as triphenylphosphine, in a solvent such as THF or ether at or about room temperature.

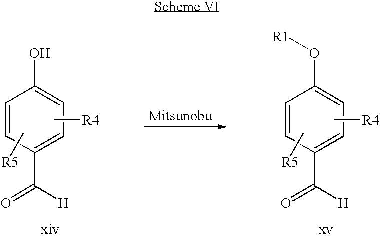

Scheme VII illustrates a method for the preparation of compounds of formula xvii, where R² to R⁵, X¹-X³ and n are defined as above, and R¹ is an optionally substituted alkyl or cycloalkyl group. Referring to scheme VII, a compound of formula xvii can be prepared from the coupling of a compound of formula R₁OH with a compound of formula xvi in the presence of a suitable coupling reagent such as diethylazodicarboxylate (DEAD) or di-tert-butylazodicarboxylate and a phosphine, such as triphenylphosphine, in a solvent such as THF or ether at or about room temperature.

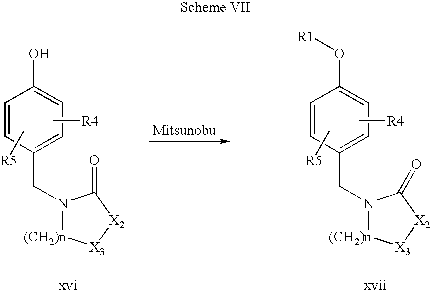

Scheme VIII illustrates a method for the preparation of compounds of formula xvii, where R² to R⁵, X¹-X³ and n are defined as above, and R¹ is an optionally substituted alkyl, heterocycloalkyl or cycloalkyl group. Referring to scheme VIII, a compound of formula xvii can be prepared from the alkylation of a compound of formula xvi with a compound of formula R₁ Cl, R₁I or R₁Br in the presence of a suitable base, such as, but not limited to, triethylamine, diisopropylethylamine, sodium hydride, sodium carbonate, cesium carbonate, potassium carbonate, potassium tert-butoxide or sodium ethoxide, in a solvent such THF, DMF, acetone or DMSO, at a temperature between room temperature and 150° C. with or without microwave heating.

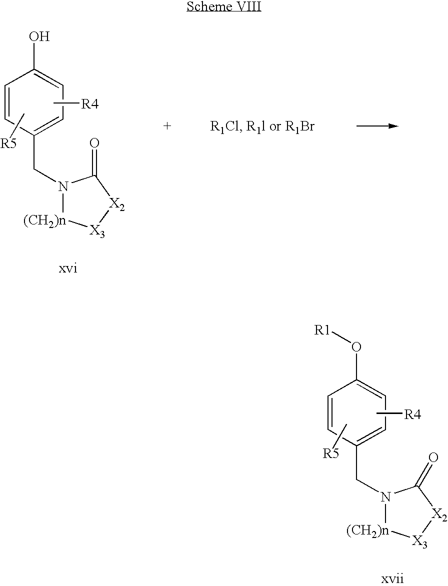

Scheme IX illustrates a method for the preparation of compounds of formula xix, where R¹, R³ to R⁵, X¹-X³ and n are defined as above, and R² is an optionally substituted alkyl, heterocycloalkyl or cycloalkyl group. Referring to scheme IX, a compound of formula xix can be prepared from the alkylation of a compound of formula xviii with a compound of formula R₂Cl, R₂l or R₂Br in the presence of a suitable base, such as, but not limited to, triethylamine, diisopropylethylamine, sodium hydride, sodium carbonate, cesium carbonate, potassium carbonate, potassium tert-butoxide or sodium ethoxide, in a solvent such THF, DMF, dichloromethane or DMSO, at a temperature between room temperature and 150° C. with or without microwave heating.

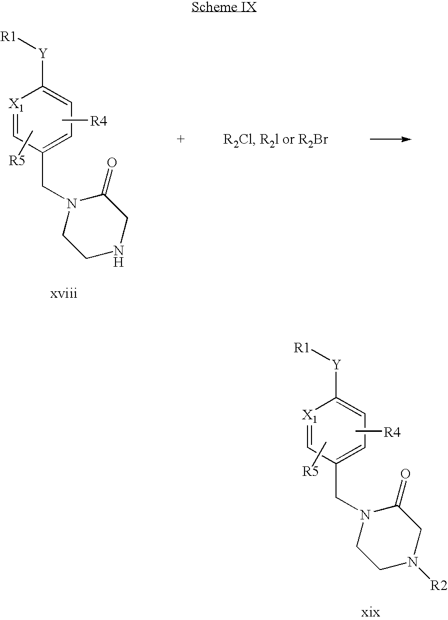

Scheme X illustrates a method for the preparation of compounds of formula xix, where R¹ to R⁵, X¹-X³ and n are defined as above. Referring to scheme X, a compound of formula xix can be synthesized by treating an amine of formula xviii with an aldehyde of formula R₂CHO in the presence of suitable reducing agents such as NaBH(OAc)₃ or Na(CN)BH₃ in solvents such as methylene chloride, dichloroethane, DMF or THF, at about room temperature. Other suitable conditions for this transformation include treatment of an amine of formula xviii with an aldehyde of formula R₂CHO in a solvent such as methanol or ethanol at room temperature, followed by treatment with a reducing agent such as NaBH₄ or NaCNBH₃, which also produce the desired compounds of formula xix.

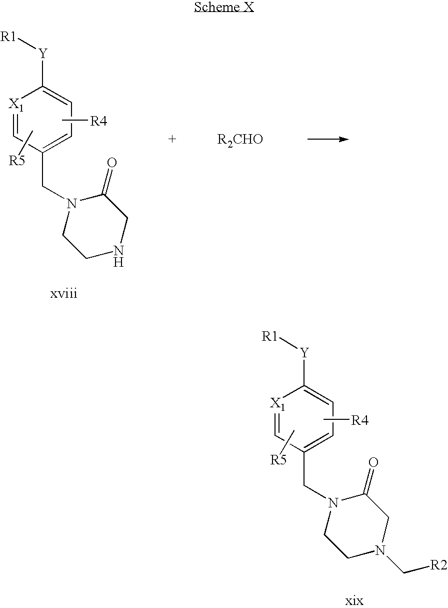

Scheme XI illustrates a method for the preparation of compounds of formula xxi, where R¹ to R⁵, X¹-X³ and n are defined as above. Referring to scheme XI, a compound of formula xxi can be synthesized by treating an amine of formula R₁R₂NH with an aldehyde of formula xx in the presence of suitable reducing agents such as NaBH(OAc)₃ or Na(CN)BH₃ in solvents such as methylene chloride, dichloroethane, DMF or THF, at about room temperature. Other suitable conditions for this transformation include treatment of an amine of formula R₁R₂NH with an aldehyde of formula xx in a solvent such as methanol or ethanol at room temperature, followed by treatment with a reducing agent such as NaBH₄ or NaCNBH₃, which also produce the desired compounds of formula xxi.

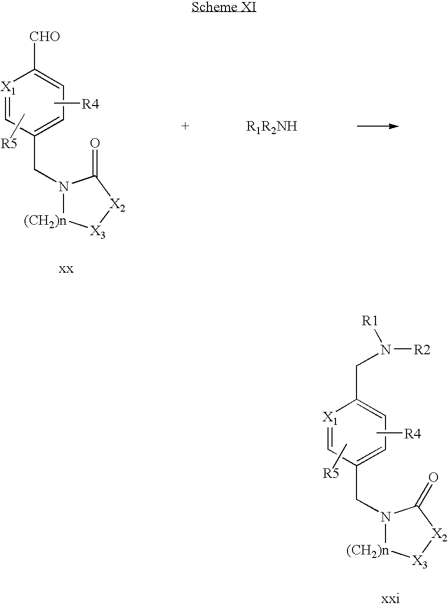

Scheme XII illustrates a method for the preparation of compounds of formula xxiii, where R¹ to R⁵, X¹-X³, Y and n are defined as above. Referring to scheme XII, a compound of formula xxiii can be prepared from the alkylation of a compound of formula R1R2NH with a compound of formula xxii in the presence of a suitable base, such as, but not limited to, triethylamine, diisopropylethylamine, sodium hydride, sodium carbonate, cesium carbonate, potassium carbonate, potassium tert-butoxide or sodium ethoxide, in a solvent such THF, DMF, dichloromethane or DMSO, at a temperature between 0° C. and 150° C. with or without microwave heating.

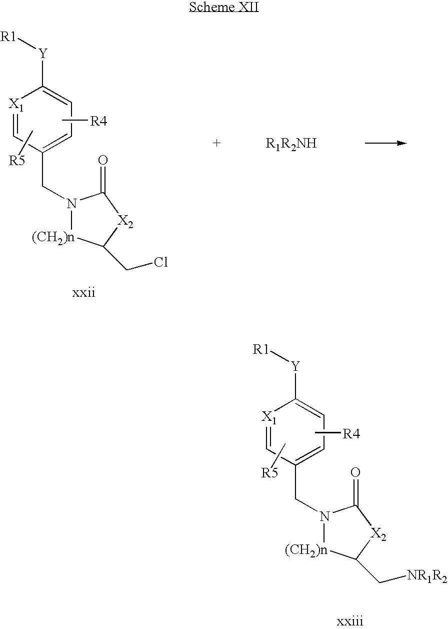

Scheme XIII illustrates a method for the preparation of compounds of formula xxv, where R² to R⁵, X¹-X³ and n are defined as above, and Ar is an optionally substituted aryl or heteroaryl group. Referring to scheme XIII, a compound of formula xxv can be prepared from the coupling of a compound of formula ArOH with a compound of formula xxiv in the presence of a suitable coupling reagent such as diethylazodicarboxylate (DEAD) or di-tert-butylazodicarboxylate and a phosphine, such as triphenylphosphine, in a solvent such as THF or ether at or about room temperature.

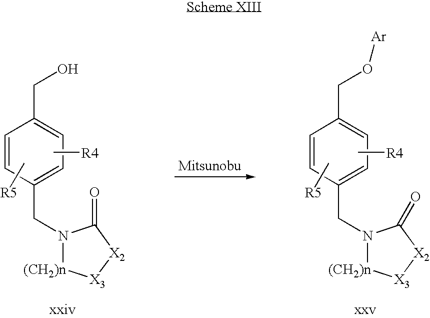

### WORKING EXAMPLES

The following illustrate the synthesis of various compounds of the present invention. Additional compounds within the scope of this invention may be prepared using the methods illustrated in these Examples, either alone or in combination with techniques generally known in the art.

### HPLC Methods

HPLC Method A

Solvent Delivery System: Waters 2795 Alliance HT

Mobile Phase A: Water; B: Acetonitrile; C: Modifier (1% Trifluoroacetic Acid) in water

| | Time | % A | % B | % C |
|---|---|---|---|---|
| Gradient: | 0 | 90 | 5 | 5 |
| | 0.2 | 90 | 5 | 5 |
| | 4 | 0 | 95 | 5 |
| | 4.7 | 0 | 95 | 5 |
| | 5 | 90 | 5 | 5 |

Flow rate: 2.0 mL/min

Column: Symmetry C8 4.6×50 mm, 3.5 um

UV Detection: Waters 996 Photodiode Array (wavelength range 200-400 nm)

Polymer Labs 2100 ELSD Parameters:

Evaporator Temp=60; Nebulizer Temp=45; Gas=1.5;

Photomultiplier=2.5; Smooth=1; LED=100

Mass Spectrometer: Waters Micromass ZQ single quadrupole MS

Electrospray Ionization mode=positive; Scan Range=160-650 da; Cone voltage=30V

HPLC Method B

Solvent Delivery System: Waters 2795 Alliance HT

Mobile Phase A: water; B: acetonitrile; C: modifier (0.6% ammonium hydroxide) in water

| | Time | % A | % B | % C |
|---|---|---|---|---|
| Gradient: | 0 | 90 | 5 | 5 |
| | 0.2 | 90 | 5 | 5 |
| | 4 | 0 | 95 | 5 |
| | 4.7 | 0 | 95 | 5 |
| | 5 | 90 | 5 | 5 |

Flow rate=2.0 mL/min

Column: XTerra C18 4.6×50 mm, 3.5 um

UV Detection: Waters 996 Photodiode Array (wavelength range 200-400 nm)

Polymer Labs 2100 ELSD Parameters:

Evaporator Temp=60; Nebulizer Temp=45; Gas=1.5; Photomultiplier=2.5; Smooth=1; LED=100

Mass Spectrometer Waters Micromass ZQ single quadrupole MS

Electrospray Ionization mode=positive; Scan Range=160-650 da; Cone voltage=30 V

HPLC Method C

LC information: guard column=Waters X-terra C-18 guard; column=Waters X-terra C18 19×50 mm 10 um; column temp.=room temperature; Flow rate (Prep+Analytical)=80 mL/min

Solvent: A (A+C=A) Water+modifier

Solvent: B Acetonitrile

Solvent: C (modifier)5% Ammonia

% Solvent C (of total) 1%

MS make up solvent=methanol/water/FA 90/10/0.1; MS make up flow rate=2 mL 1 min; online dilution solvent=acetonitrile; online dilution flow rate, =8 mL min.

Mobile Phase path information: No post column modifier; flow split in MS (yes)

Sample Info. Injection solvent=DMSO; injection volume=1 mL; amount of sample=100 umoles

Detection Information:

MS Manufacturer/Model=Waters ZQ; ionization mode=ESI+; mass range=190-800 amu; cone voltage (V)=20; capillary voltage (V)=3.3; source temp.=140° C.; UV model=Shimadzu SPD 10A; desolvation Temp.=350° C.; Wavelength=254 nm; nebulization gas=100 L/h; desolvation gas=600 L/h

HPLC Method D

Analytical Conditions:

Column=Waters SunFire C18, 5 um, 3.0×50 mm steel column, part #186002545

Solvent A: Water

Solvent B: Acetonitrile

Solvent C: 10% Trifluoroacetic acid/Water

Solvent D: 10% Ammonium hydroxide/Water

| Time | A (%) | B (%) | C (%) | D (%) | Flow |
|---|---|---|---|---|---|
| 0.0 | 94.0 | 5.0 | 1.0 | 0.0 | 1.6 |
| 1.0 | 94.0 | 5.0 | 1.0 | 0.0 | 1.6 |
| 6.0 | 4.0 | 95.0 | 1.0 | 0.0 | 1.6 |
| 7.0 | 4.0 | 95.0 | 1.0 | 0.0 | 1.6 |

Preparative Conditions:

Column=Waters SunFire Prep C18 OBD, 5 um, 19×100 mm steel column, part #186002567

Solvent A: water

Solvent B: acetonitrile

Modifier: 1% trifluoroacetic acid/water

Makeup Solvent: 2 mM ammonium formate/80% methanol/water

| Time | A (%) | B (%) | Flow | Modifier Flow |
|---|---|---|---|---|
| 0.0 | 95.0 | 5.0 | 18.0 | 2.0 |
| 1.0 | 95.0 | 5.0 | 18.0 | 2.0 |
| 2.0 | 70.0 | 30.0 | 18.0 | 2.0 |
| 5.0 | 40.0 | 60.0 | 18.0 | 2.0 |
| 6.0 | 5.0 | 100.0 | 18.0 | 2.0 |
| 7.0 | 5.0 | 100.0 | 18.0 | 2.0 |

Mass Spect Parameters: capillary voltage (kV)=3.0; cone voltage (V)=20.0; extractor voltage (V)=3.0; RF lens voltage (V)=0.5; source temp. (Celsius)=120.0; desolvation temp. (Celsius)=360.0; desolvation gas flow (L/hr.)=450.0; cone gas flow (L/hr.)=150.0; LM resolution=15.0; HM resolution=15.0; ion energy=0.5; multiplier=600.0; ion mode=ES+; data format=Centroid; start mass=150; end mass=700; prep start mass=250; prep end mass=450; scan time (sec.)=0.5; interscan time (sec.)=0.1; start time (min.)=0.0; end time (min.)=7.0; start wavelength=200; end wavelength=700

ELSD parameters (Polymer Labs ELS-2100) and PDA parameters (Waters 996): evaporator temp.=60° C.; start wavelength=200; nebuliser temp.=45° C.; end wavelength=700; gas flow (SLM)=1.6; resolution (nm)=1; sampling rate (spec/s)=1

HPLC Method E

Solvent Delivery System: Gilson 215 with 306 pumps

Mobile Phase: A: 98% water, 2% acetonitrile, 0.01% formic acid
B: acetonitrile with 0.005% formic acid

| | Time | % A | % B |
|---|---|---|---|
| Gradient: | 0 | 95 | 5 |
| | 1.05 | 80 | 20 |
| | 2.3 | 50 | 50 |
| | 3.55 | 0 | 100 |
| | 3.76 | 95 | 5 |

Flow rate=1.0 mL/min; column: Polaris C18-A-20×2.0 mm

UV Detection: Hewlett Packard Series 1100

Wavelength Range=200-400 nm

Mass Spectrometer Waters Micromass ZQ single quadrupole MS

Electrospray ionization mode=positive; scan range=160-1100 da; cone voltage=20-30 V

HPLC Method F

Column=Waters XBridge MS C18, 5 μm, 3.0×50 mm steel column, part #186003131

Solvent A: Water

Solvent B: Acetonitrile

Solvent C: 10% Trifluoroacetic acid/Water

Solvent D: 10% Ammonium hydroxide/Water

| Time | A (%) | B (%) | C (%) | D (%) | Flow |
|---|---|---|---|---|---|
| 0.0 | 94.0 | 5.0 | 0 | 1.0 | 1.6 |
| 1.0 | 94.0 | 5.0 | 0 | 1.0 | 1.6 |
| 6.0 | 4.0 | 95.0 | 0 | 1.0 | 1.6 |
| 7.0 | 4.0 | 95.0 | 0 | 1.0 | 1.6 |

Preparative Conditions:

Column=Waters XBridge Prep C18 OBD, Sum, 19×100 mm steel column, part #186002978

Solvent A: Water

Solvent B: Acetonitrile

Modifier: 1% Trifluoroacetic acid/Water

Makeup Solvent: Methanol

| Time | A (%) | B (%) | Flow | Modifier Flow |
|---|---|---|---|---|
| 0.0 | 95 | 5 | 18.0 | 2.0 |
| 1.0 | 95 | 5 | 18.0 | 2.0 |
| 5.0 | 5 | 95 | 18.0 | 2.0 |
| 7.0 | 5 | 95 | 18.0 | 2.0 |

Mass Spect Parameters: capillary voltage (kV)=3.0; cone voltage (V)=20.0; extractor voltage (V)=3.0; RF lens voltage (V)=0.5; source temp. (Celsius)=120.0; desolvation temp. (Celsius)=360.0; desolvation gas flow (L/hr.)=450.0; cone gas flow (L/hr.)=150.0; LM resolution=15.0; HM resolution=15.0; ion energy=0.2; multiplier=600.0; ion mode=ES+; data format=Centroid; start mass=150; end mass=700; prep start mass=150; prep end mass=800; scan time (sec.)=0.5; interscan time (sec.)=0.1; start time (min.)=0.0; end time (min.)=9.0; start wavelength=200; end wavelength=700

ELSD parameters (Polymer Labs ELS-2100) and PDA parameters (Waters 996): evaporator temp.=60° C.; start wavelength=200; nebuliser temp.=45° C.; end wavelength=700; gas flow (SLM)=1.6; resolution (nm)=1; sampling rate (spec/s)=1

Examples 1-56 were prepared in library format as follows:

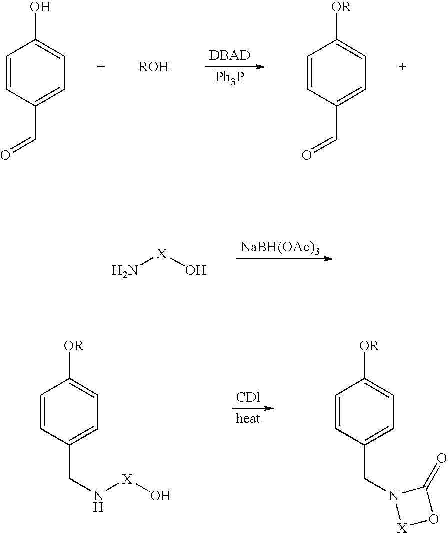

These compounds were prepared in library format using standard parallel chemistry techniques and automation.

Step 1—Mitsunobu Reaction

Solutions of alcohols used as the diversity element (0.5 mL of a 0.53M solution in anhydrous THF, 0.27 mmol, 2.7 eq.) were transferred to 8 mL round-bottom vials fitted with septum caps. Di-tert-butylazodicarboxylate (0.5 mL of a 0.4M solution in anhydrous THF, 0.2 mmol, 2 eq.) was added to each vial, and the reactions were shaken at room temperature. After 25 min, Ph₃P (0.5 mL of a 0.5M solution in anhydrous THF, 0.25 mmol, 2.5 eq.) was added to each vial, and the reactions were shaken at room temperature for 25 min. 4-Hydroxybenzaldehyde (0.5 mL of a 0.2M solution in anhydrous THF, 0.1 mmol, 1 eq.) was added, and the reactions were shaken at room temperature for 18 h. THF was removed under a stream of N₂ at room temperature.

Step 2—Reductive Amination

Crude 4-substituted benzaldehydes were dissolved in 1 mL 1,2-dichloroethane. Aminoalcohols (250 uL of a 0.4M solution in DCE, 0.1 mmol, 1 eq.) were added (with an equivalent of DIEA to neutralize salts if necessary), then NaBH(OAc)₃ (500 uL of a 0.6M suspension in CHCl₃, 0.3 mmol, 3 eq.) was added to each reaction. The vials were covered and shaken at room temperature for 18 h. The reactions were quenched with 1 mL 10% NH₄OH, vortexed vigorously for 5 min, and the phases allowed to settle for 10 min. The biphasic mixtures were loaded onto Varian Hydromatrix (ChemElut) cartridges (1 mL aq. capacity), allowed to stand for 5 min, and eluted with (2×3 mL) DCE into clean 8 mL round bottomed vials. The solvent was removed under a stream of N₂ at room temperature.

The crude products were dissolved in 1 mL MeOH and transferred to Waters Oasis MCX cartridges (6 mL, 400 mg sorbent) which had been conditioned with 1 mL MeOH. The vials were washed with 2 mL MeOH, which was also transferred to cartridges. The cartridges were washed with a final aliquot of 3 mL MeOH, and then the product was eluted with 5 mL 1M NH₃/MeOH into clean 8 mL round bottomed vials.

Step 3—Formation of Cyclic Carbamates

The crude aminoalcohol products were dissolved in 500 uL THF, then 1,1-carbonyldiimidazole (500 uL of a 0.2M solution in THF, 0.1 mmol, 1 eq.) was added to every vial. The vials were capped securely and heated to 80° C. for 3.5 h. THF was removed under a stream of N₂ at room temperature. The residue was partitioned between 1 mL DCE/1 mL 50% saturated NH₄Cl, then the biphasic mixture was loaded onto Varian Hydromatrix (ChemElut) cartridges (1 mL aq. capacity) and allowed to stand for 5 min. The cartridges were eluted with (2×3 mL) DCE into tarred 8 mL round bottomed vials. The reactions were dried and crude weights taken. The crude products were dissolved in 1000 uL DMSO, and purified via high-throughput preparative LC/MS (Sunfire C18 19×100 mm, 5 um column, acetonitrile/water gradient with 1% TFA, toluene and ethanol as azeotroping solvents). Post-purification analysis was performed using HPLC methods A and B.

### Example 57

### (5R)-3-[(4′-isopropoxybiphenyl-4-yl)methyl]-5-methyl-1,3-oxazolidin-2-one

A mixture of 4′-isopropoxybiphenyl-4-carbaldehyde (1.1 g, 4.6 mmol), (R)-(−)-1-amino-2-propanol (0.4 mL, 5.1 mmol) and sodium triacetoxyborohydride (3.4 g, 16 mmol) in dichloromethane (25 mL) was stirred at room temperature. After 18 h the mixture was diluted with 10% aqueous ammonium hydroxide and extracted three times into dichloromethane. The combined organics were dried over sodium sulfate and concentrated under reduced pressure to give 1.3 g of a white solid. The solid was dissolved in 25 mL of anhydrous THF, carbonyl diimidazole (0.7 g, 4.3 mmol) was added and the mixture was stirred at 80° C. After 3.5 h the mixture was cooled to room temperature, saturated ammonium chloride was added and the resulting mixture was extracted 3 times with dichloromethane. The combined organics were concentrated under reduced pressure and purified by silica gel chromatography eluting with 20% ethyl acetate in hexanes to give 800 mg of the title compound as a white solid.

Example 58-64 were prepared from the appropriate biphenyl aldehydes using the same procedure as described in example 57

### Example 65

### (5R)-3-[4(1-cyclohexylethoxy)benzyl]-5-methyl-1,3-oxazolidin-2-one

To a stirred mixture of 4-(1-cyclohexylethoxy)benzaldehyde (50 mg, 0.22 mmol) and (R)-(−)-1-amino-2-propanol (18 mg, 0.24 mmol) in 1 mL of dichloromethane was added a slurry of sodium triacetoxyborohydride (160 mg, 0.75 mmol) in 1 mL dichloromethane. After stirring for 18 h at room temperature, 10% ammonium hydroxide was added and the mixture was concentrated under reduced pressure. The resulting residue was dissolved in 2 mL of methanol and loaded onto an Oasis MCX cartridge which had been pre-conditioned with 1 mL of methanol. The cartridge was washed twice with 2 mL of methanol and then flushed with a 2M solution of ammonia in methanol. The collected fractions from the ammonia/methanol wash were combined and concentrated to give 18 mg of a white solid (MS m/z 292.2). The solid (17 mg, 0.06 mmol) was dissolved in 1 mL of THF and carbonyldiimidazole (9.4 mg, 0.06 mmol) was added. The mixture was stirred under nitrogen at 80° C. After 4 h the mixture was diluted with saturated aqueous ammonium chloride and extracted into dichloromethane. The combined organics were concentrated and purified by silica gel chromatography eluting with 20% ethyl acetate in hexanes to give 9 mg of the title compound as a colorless oil.

Examples 66-70 were prepared from the reaction of either (R)-(−)-1-amino-2-propanol or (S)-(+)-1-amino-2-propanol with the appropriate 4-alkoxyaldehyde using the same procedure as described in example 65.

### Example 82

### (5R)-3-Biphenyl-4-ylmethyl)-5-methyl-1,3-oxazolidin-2-one

To a vial containing phenylboronic acid (99 mg, 0.8 mmol) and sodium carbonate (40 mg, 0.4 mmol) was added a solution of (5R)-3-(4-bromobenzyl)-5-methyl-1,3-oxazolidin-2-one (50 mg, 0.19 mmol) in 0.4 mL of ethanol followed by a solution of tetrakis(triphenylphosphine)palladium(0) (21 mg, 0.02 mmol) in 0.1 mL of toluene. The reaction was stirred and heated in a microwave at 120° C. for 5 min. The reaction mixture was then diluted with 10% ammonium hydroxide, extracted into ethyl acetate and the combined organics were concentrated under reduced pressure. Purification by chromatography on a silica gel column eluting with 1:9 ethanol/heptane provided 18 mg of the title compound as a pale yellow amorphous solid.

Examples 83-86 were prepared from (5R)-3-(4-bromobenzyl)-5-methyl-1,3-oxazolidin-2-one and the appropriate arylboronic acid using the procedure described in example 82.

### Example 87

### (5R)-3-[(2,2-Dimethyl-3,4-dihydro-2H-chromen-6-yl)methyl]-5-methyl-1,3-oxazolidin-2-one

Prepared from 2,2-dimethylchroman-6-carbaldehyde using the procedure described in example 57.

Examples 88-152 were prepared in library format using the same procedures as described in examples 1-56. For examples 88-152 racemic 1-amino-2-propanol was the aminoalcohol used in the reductive amination step (step 2).

Examples 153-177 were prepared in library format as follows using standard parallel chemistry techniques:

A microwave safe tube containing 0.2 mmol of sodium carbonate and 0.4 mmol of the appropriate boronic acid was treated with 0.7 mL of a 0.1 mM solution of (5R)-3-(4-bromobenzyl)-5-methyl-1,3-oxazolidin-2-one in ethanol. The tube was capped with a septum, purged with nitrogen, and 0.1 mL of a 0.1 M solution of tetrakistriphenylphosphine palladium(0) in toluene was added. The reaction mixture was stirred at 120° C. for 5 min in a microwave. The reaction mixture was cooled to room temperature and then diluted with 1.5 mL ethyl acetate and 1 mL of 1N NaOH, and vortexed. The organic layer was removed and the aqueous layer was extracted two times with ethyl acetate. The combined organics were passed through cartridges of sodium sulfate and the resulting solutions were concentrated under reduced pressure. The resulting crude reaction mixtures were purified by preparative HPLC using the conditions described in method D.

### Example 178

### (5R)-3-[4-(2,5-dimethyl-1H-pyrrol-1-yl)benzyl]-5-methyl-1,3-oxazolidin-2-one

Prepared from 4-(2,5-dimethyl-1H-pyrrol-1-yl)benzaldehyde using the procedure described in example 57.

Examples 179-186 were prepared in library format as follows using standard parallel chemistry techniques:

To a vial containing the appropriate aldehyde (0.25 mmol) was added a solution of R-(−)-1-amino-2-propanol (22 └, 0.28 mmol) in 750 └dichloroethane followed by solid sodium triacetoxyborohydride (185 mg, 3.5 equiv.). After stirring for 3 days at room temperature the reaction mixture was diluted with 1.5 mL 1N NaOH and extracted 3× into ethyl acetate. The combined organics were passed through a SCX cartridge, and flushed with 1 mL of methanol. The product was collected by flushing the cartridge with a 9:1 mixture of methanol and triethylamine. The resulting solution was then concentrated under reduced pressure to provide a residue which was assumed to be the expected aminoalcohol intermediate. The aminoalcohol intermediates were treated with a solution of carbonyl diimidazole (40 mg, 0.25 mM) in 0.75 mL anhydrous THF and were then shaken at 80° C. After 3.5 h the reaction vials were diluted with 2.5 mL of ethyl acetate and 1 mL of half-saturated aqueous ammonium chloride, vortexed, and allowed to settle for 10 min. The organic phase was separated and the aqueous layer was extracted 2× with ethyl acetate. The combined organics were passed through a sodium sulfate cartridge, concentrated under reduced pressure and purified by preparative HPLC using method D.

### Example 187

### (5R)-3-[(3′,5′-dichlorodiphenyl-4-yl)methyl]-5-methyl-1,3-oxazolidin-2-one

Prepared from 3,5-dichlorophenylboronic acid and (5R)-3-(4-bromobenzyl)-5-methyl-1,3-oxazolidin-2-one using the procedure described in example 82.

### Example 188

### 1-[4-(1-cyclohexylethoxy)benzyl]-4-ethylpiperazine-2-one

To a solution of 1-(4-(1-cyclohexylethoxy)benzyl)piperazin-2-one hydrochloride (77.4 mg) in 3 mL of dichloromethane, acetaldehyde (96.7 mg), magnesium sulfate (20 mg) and triethyl amine (0.15 mL) were added sequentially and the mixture was stirred for one hour at room temperature. Then, sodium triacetoxyborohydride (116.3 mg) was added into the mixture. The mixture was stirred at room temperature for 16 h and was then filtered and purified by preparative HPLC to yield 4 mg of the title compound as a trifluoroacetic acid salt (4.0 mg).

Example 189-199 were prepared from (5R)-3-(4-bromobenzyl)-5-methyl-1,3-oxazolidin-2-one and the appropriate arylboronic acid using the procedure described in example 82.

Examples 200-202 were prepared from 1-(4-(1-cyclohexylethoxy)benzyl)piperazin-2-one hydrochloride and the appropriate aldehyde using the procedure described in example 188.

### Examples 203

### (5R)-3-[4-(Cyclohexylmethoxy)benzyl]-5-methyl-1,3-oxazolidin-2-one

To a stirred mixture of 4-(cyclohexylmethoxy)benzaldehyde (560 mg, 2.6 mmol) and (R)-(−)-1-amino-2-propanol (210 mg, 2.8 mmol) in 10 mL of dichloromethane was added sodium triacetoxyborohydride (1.9 g, 9.0 mmol). After stirring for 16 h at room temperature, 50% ammonium hydroxide was added and the mixture extracted 3× with dichloromethane. The combined organics were washed with brine, dried over magnesium sulfate and concentrated to give 200 mg of an amorphous solid (MS m/z 278.4). The solid (200 mg) was dissolved in 5 mL of THF and carbonyldiimidazole (117 mg, 0.72 mmol) was added. The mixture was stirred under nitrogen at 80° C. After 4 h the mixture was diluted with saturated aqueous ammonium chloride and extracted into ethyl acetate. The combined organics were concentrated and purified by silica gel chromatography eluting with 9:1 heptane/ethanol to give 120 mg of the title compound as a white solid

Examples 204-206 were prepared from the reaction of (R)-(−)-1-amino-2-propanol with the appropriate 4-alkoxyaldehyde using the procedure described in example 203.

Examples 207-209 were prepared from the reaction of 2-amino-1-(2-pyridyl)ethanol, 2-amino-1-(3-pyridyl)ethanol, and 2-amino-1-(4-pyridyl)ethanol, respectively, with 4-(1-cyclohexylethoxy)benzaldehyde using the procedure described in example 203.

### Example 210

### (5R)-3-{[6-(4-Chloro-2-fluorophenyl)pyridin-3-yl]methyl}-5-methyl-1,3-oxazolidin-2-one

To a mixture of (5R)-3-[(6-chloropyridin-3-yl)methyl]-5-methyl-1,3-oxazolidin-2-one (50 mg, 0.22 mmol), 4-chloro-2-fluorophenylboronic acid (38 mg, 0.22 mmol) and sodium carbonate (50 mg, 0.44 mmol) in ethanol in a glass vial was added tetrakistriphenylphosphine palladium(0) (25 mg, 0.022 mmol). After stirring for 10 min at 150° C. in a microwave oven the mixture was cooled to room temperature, filtered and concentrated under reduced pressure. The residue was partitioned between 1N HCl and ethyl acetate, and the aqueous layer was basified with 1N NaOH and extracted with ethyl acetate. The combined organics were washed with brine, dried over sodium sulfate and concentrated under reduced pressure. Purification by chromatography on a silica gel column eluting with a gradient of 30-80% of (19:1 ethyl acetate/methanol) in hexanes provided 20 mg of the title compound as a transparent oil.

### Example 211

### (5R)-3-{[6-(1-cyclohexylethoxy)pyridin-3-yl]methyl}-5-methyl-1,3-oxazolidin-2-one

To a stirred mixture of (5R)-3-[(6-chloropyridin-3-yl)methyl]-5-methyl-1,3-oxazolidin-2-one (46 mg, 0.20 mmol) and 1-cyclohexylethanol (26 mg, 0.20 mmol) in 2 mL of THF was added 0.3 mL of a 1M solution of tert-butoxide in THF. After heating in a microwave oven for 10 min at 120° C. the reaction mixture was cooled to room temperature, diluted with water and extracted 2× with 30 mL of ethyl acetate. The combined organics were washed with brine, dried over sodium sulfate, and concentrated under reduced pressure. Purification by chromatography on silica gel eluting with a gradient of 40-80% of (19:1 ethyl acetate/methanol) in hexanes provided 10 mg of the title compound as a transparent oil.

Examples 212-213 were prepared from the reaction of (R)-(−)-1-amino-2-propanol with the appropriate 4-alkoxyaldehyde using the same procedure as described in example 203.

Examples 214-225 were prepared in library format as follows using standard parallel chemistry techniques:

A solution of 1-(4-(1-cyclohexylethoxy)benzyl)piperazin-2-one hydrochloride (25 mg, 0.07 mmol) and triethylamine (20 └L) in 750 └L dichloromethane was added to 5 equiv. of the appropriate ketone/aldehyde followed by 10 mg of magnesium sulfate. The mixture was shaken at room temperature for 1 h, and then sodium triacetoxyborohydride (55 mg, 3.5 equiv., 0.25 mmol) was added and the reaction mixtures were shaken at room temperature. After 24 h 1N sodium hydroxide (1.5 mL) was added to each reaction mixture and the resulting mixture was extracted (2.5 mL×3) with ethyl acetate. The combined organics were passed through a sodium sulfate cartridge and then concentrated under reduced pressure. The residues were dissolved in 0.5 mL of 10% trifluoroacetic acid (TFA) in dichloromethane and then concentrated under reduced pressure to provide the corresponding TFA salts. Purification by preparative HPLC using the conditions described in HPLC Method F provided the title compounds.

### Example 226

(5R)-3-{[2′-Fluoro-4′-(trifluoromethoxy)biphenyl-4-yl]methyl}-5-methyl-1,3-oxazolidin-2-one was prepared from (5R)-3-(4-bromobenzyl)-5-methyl-1,3-oxazolidin-2-one and 2-fluoro-4-trifluoromethoxyboronic acid using the procedure described in example 82.

### Example 227

### (5R)-3-[4-(cyclohexylmethoxy)-3-fluorobenzyl]-5-methyl-1,3-oxazolidin-2-one

A mixture of (5R)-3-(4-{[tert-butyl(dimethyl)silyl]oxy}-3-fluorobenzyl)-5-methyl-1,3-oxazolidin-2-one (20 mg, 0.05 mmol) and potassium fluoride (4 mg) in 1 mL of THF and 1 mL of DMF was heated in a microwave oven at 120° C. for 20 min (MS m/z 226.3, retention time 1.1 min). After cooling to room temperature 0.05 mL of a 0.1 M solution of potassium tert-butoxide was added followed by (bromomethyl)cyclohexane (10 mg, 0.55 mmol) and the mixture was heated in a microwave oven at 120° C. for 20 min. Water was added and the mixture was extracted with ethyl acetate. The combined organics were washed with brine, dried over sodium sulfate and concentrated under reduced pressure. Purification by preparative TLC eluting with 1:1 ethyl acetate/hexanes provided 5 mg of the title compound.

### Example 228

(5R)-3-(3-fluoro-4-{[2-fluoro-4-(trifluoromethyl)benzyl]oxy}benzyl)-5-methyl-1,3-oxazolidin-2-one was prepared from (5R)-3-(4-{[tert-butyl(dimethyl)silyl]oxy}-3-fluorobenzyl)-5-methyl-1,3-oxazolidin-2-one and 2-fluoro-4-trifluoromethylbenzyl bromide using the procedure described in example 227.

### Example 229

(5R)-3-({6-[3-(4-fluorophenoxy)propoxy]pyridin-3-yl}methyl)-5-methyl-1,3-oxazolidin-2-one was prepared from (5R)-3-[(6-chloropyridin-3-yl)methyl]-5-methyl-1,3-oxazolidin-2-one and 3-(4-fluorophenoxy)propanol using the procedure described in example 211.

### Example 230

### (5R)-3-[4-(1-Cyclohexylethoxy)-3-fluorobenzyl]-5-methyl-1,3-oxazolidin-2-one

A mixture of (5R)-3-(3-fluoro-4-hydroxybenzyl)-5-methyl-1,3-oxazolidin-2-one (50 mg, 0.22 mmol), 1-cyclohexylethanol (56 mg, 0.44 mmol), di-tert-butyl azodicarboxylate (101 mg, 0.4 mmol) and triphenylphosphine (105 mg, 0.4 mmol) in anhydrous THF was stirred at room temperature under nitrogen. After 16 h water was added and the resulting mixture was extracted 3× into ethyl acetate. The combined organics were washed with brine, dried over sodium sulfate and concentrated under reduced pressure. Purification by silica gel chromatography eluting with a gradient of 30-100% ethyl acetate in heptane provided 30 mg of the title compound as a transparent oil.

Examples 231-240 were prepared from (5R)-3-(4-bromobenzyl)-5-methyl-1,3-oxazolidin-2-one and the appropriate arylboronic acid using the procedure described in example 82.

### Example 241

(5R)-3-[4-(2-cyclohexylethoxy)-3-fluorobenzyl]-5-methyl-1,3-oxazolidin-2-one was prepared from (5R)-3-(4-{[tert-butyl(dimethyl)silyl]oxy}-3-fluorobenzyl)-5-methyl-1,3-oxazolidin-2-one and 1-bromo-2-cyclohexylmethane using the procedure described in example 227.

Examples 242-248 were prepared from (5R)-3-(4-bromobenzyl)-5-methyl-1,3-oxazolidin-2-one and the appropriate arylboronic acid using the procedure described in example 82.

### Example 249

(5R)-5-methyl-3-[(2′,3,4′-trifluorobiphenyl-4-yl)methyl]-1,3-oxazolidin-2-one was prepared from (5R)-3-(4-bromo-2-fluorobenzyl)-5-methyl-1,3-oxazolidin-2-one and 2,4-difluorophenylboronic acid using the procedure described in example 82.

### Examples 250

### 3-[4-(Cyclohexylmethoxy)-3-methylbenzyl]-5-pyridin-3-yl-1,3-oxazolidin-2-one

To a stirred mixture of 4-(cyclohexylmethoxy)-3-methylbenzaldehyde (100 mg, 0.43 mmol) and 2-amino-1-(3-pyridyl)ethanol (59 mg, 0.43 mmol) in 3 mL of dichloromethane was added sodium triacetoxyborohydride (182 mg, 0.86 mmol). After stirring for 16 h at room temperature, 50% ammonium hydroxide was added and the mixture extracted 3× with dichloromethane. The combined organics were washed with brine, dried over sodium sulfate and concentrated to give 80 mg of an amorphous solid (MS m/z 355.5). The solid (50 mg) was dissolved in 2 mL of THF and carbonyldiimidazole (23 mg, 0.14 mmol) was added. The mixture was stirred under nitrogen at room temperature. After 16 h the mixture was concentrated under reduced pressure, water was added and the resulting mixture was extracted with ethyl acetate. The combined organics were washed with brine, dried over sodium sulfate, concentrated and purified by silica gel chromatography eluting with a gradient of ethyl acetate and hexanes to give 3 mg of the title compound as an amorphous solid.

Examples 251-253 were prepared from (5R)-3-(4-bromo-2-fluorobenzyl)-5-methyl-1,3-oxazolidin-2-one and the appropriate boronic acid using the procedure described in example 82.

### Example 254

(5R)-3-[4-(Cyclohexylmethoxy)-3-methylbenzyl]-5-methyl-1,3-oxazolidin-2-one was prepared from (5R)-3-(4-{[tert-butyl(dimethyl)silyl]oxy}-3-methylbenzyl)-5-methyl-1,3-oxazolidin-2-one and cyclohexylmethyl bromide using the procedure described in example 227.

Examples 255-282 were prepared from either (5R)-3-(4-bromobenzyl)-5-methyl-1,3-oxazolidin-2-one or (5R)-3-[(6-chloropyridin-3-yl)methyl]-5-methyl-1,3-oxazolidin-2-one and the appropriate arylboronic acid using the procedure described in example 82. Purification of these examples was performed using preparative TLC.

### Example 283

### (5R)-3-({6-[(Cyclohexylmethyl)amino]pyridin-3-yl}methyl)-5-methyl-1,3-oxazolidin-2-one

A mixture of (5R)-3-[(6-chloropyridin-3-yl)methyl]-5-methyl-1,3-oxazolidin-2-one (70 mg) and cyclohexylethylamine (0.1 mL) in a glass tube was heated in a microwave at 150° C. After 40 min the mixture was cooled to room temperature, diluted with dichloromethane and purified by chromatography on a silica gel column eluting with a gradient of 50% to 90% ethyl acetate in heptane to give 20 mg of the title compound as a transparent oil.

### Examples 284

(5R)-5-Methyl-3-(3-methyl-4-{[4-(trifluoromethyl)benzyl]oxy}benzyl)-1,3-oxazolidin-2-one was prepared from (5R)-3-(4-{[tert-butyl(dimethyl)silyl]oxy}-3-methylbenzyl)-5-methyl-1,3-oxazolidin-2-one and 4-(trifluoromethyl)benzyl bromide using the procedure described in example 227.

### Examples 285

(5R)-3-(4-{[4-fluoro-2-(trifluoromethyl)benzyl]oxy}-3-methylbenzyl)-5-methyl-1,3-oxazolidin-2-one was prepared from (5R)-3-(4-{[tert-butyl(dimethyl)silyl]oxy}-3-methylbenzyl)-5-methyl-1,3-oxazolidin-2-one and 4-fluoro-2-(trifluoromethyl)benzyl bromide using the procedure described in example 227.

### Examples 286

(5R)-3-[3-chloro-4-(cyclohexylmethoxy)benzyl]-5-methyl-1,3-oxazolidin-2-one was prepared from (5R)-3-(4{[tert-butyl(dimethyl)silyl]oxy}-3-chlorobenzyl)-5-methyl-1,3-oxazolidin-2-one and cyclohexylmethyl bromide using the procedure described in example 227.

### Examples 287

(5R)-3-[3-chloro-4-(2-cyclohexylethoxy)benzyl]-5-methyl-1,3-oxazolidin-2-one was prepared from (5R)-3-(4-{[tert-butyl(dimethyl)silyl]oxy}-3-chlorobenzyl)-5-methyl-1,3-oxazolidin-2-one and 1-bromo-2-cylcohexylethane using the procedure described in example 227.

Examples 288-295 were prepared from 2-fluoro-4-{[(5R)-5-methyl-2-oxo-1,3-oxazolidin-3-yl]methyl}phenyl trifluoromethanesulfonate and the appropriate boronic acid using the procedure described in example 82.

### Example 296

(5R)-3-{4-[(3,5-Dichloropyridin-2-yl)oxy]-3-methylbenzyl}-5-methyl-1,3-oxazolidin-2-one was prepared from (5R)-3-(4-([tert-butyl(dimethyl)silyl]oxy-3-methylbenzyl)-5-methyl-1,3-oxazolidin-2-one and 2,3,5-trichloropyridine using the procedure described in example 227.

### Example 297

### (5R)-3-(4-{[4-(Cyclobutylamino)cyclohexyl]methoxy}benzyl)-5-methyl-1,3-oxazolidin-2-one

To a stirred solution of (5R)-5-methyl-3-{4-[(4-oxocyclohexyl)methoxy]benzyl}-1,3-oxazolidin-2-one (20 mg, 0.06 mmol) in methanol (2 mL) at room temperature was added cyclobutylamine (5 mg, 0.07 mmol). After 30 min sodium borohydride (4.5 mg, 0.12 mmol) was added. After 1 h the mixture was concentrated under reduced pressure and then partitioned between 1N sodium hydroxide and dichloromethane. The organic layer was washed with brine, dried over sodium sulfate and concentrated under reduced pressure. Purification by chromatography on a silica gel column eluting with 5% methanol in chloroform gave 4 mg of the title compound as a sticky amorphous solid.

### Example 298

### (5R)-3-{[6-(2,3-Difluorophenyl)-5-fluoropyridin-3-yl]methyl}-5-methyl-1,3-oxazolidin-2-one

To a stirred solution of (5R)-3-{[5-amino-6-(2,3-difluorophenyl)pyridin-3-yl]methyl}-5-methyl-1,3-oxazolidin-2-one (19 mg, 0.06 mmol) in 1 mL of 70% HF in pyridine at 0° C. was added NaNO₂ (38 mg, 0.53 mmol). After 30 min 4 mL of a 1:1 mixture of ammonium hydroxide and water was added and the mixture was extracted with ethyl acetate. The combined organics were washed with brine, dried over sodium sulfated and concentrated under reduced pressure. Purification by chromatography on a silica gel column eluting with 40% to 100% ethyl acetate in heptane provided 3 mg of the title compound as a clear oil.

### Example 299

(5R)-3-[(2′,4′-Difluoro-2-methylbiphenyl-4-yl)methyl]-5-methyl-1,3-oxazolidin-2-one was prepared from 2-methyl-4-{[(5R)-5-methyl-2-oxo-1,3-oxazolidin-3-yl]methyl}phenyl trifluoromethanesulfonate and 2,4-difluorophenylboronic acid using the procedure described in example 82.

### Example 300

(5R)-3-{4-[(5-Chloro-2,3-dihydro-1H-indol-1-yl)methyl]benzyl}-5-methyl-1,3-oxazolidin-2-one was prepared from 4-[(5-chloro-2,3-dihydro-1H-indol-1-yl)methyl]benzaldehyde and (R)-(−)-1-amino-2-propanol using the procedure described in example 203.

### Example 301

### (5R)-3-[4-(2,3-Dihydro-1H-indol-1-ylmethyl)benzyl]-5-methyl-1,3-oxazolidin-2-one

To a stirred solution of 4-{[(5R)-5-methyl-2-oxo-1,3-oxazolidin-3-yl]methyl} benzaldehyde (30 mg, 0.14 mmol) and indoline (16.3 mg, 0.14 mmol) in 3 mL of dichloromethane was added sodium triacetoxyborohydride (102 mg, 0.48 mmol) at room temperature. After 18 h the mixture was diluted with 5 mL of 50% conc. ammonium hydroxide and stirred for 30 min. The organic layer was concentrated under reduced pressure and purified by preparative TLC on a silica gel plate eluting with 1:1 heptane/ethyl acetate to give 24 mg of the title compound as a waxy, red-brown solid.

### Example 302

3-[(2′,4′-Difluorobiphenyl-4-yl)methyl]-5-pyridin-3-yl-1,3-oxazolidin-2-one was prepared from 3-(4-bromobenzyl)-5-pyridin-3-yl-1,3-oxazolidin-2-one and 2,4-difluorophenylboronic acid using the procedure described for example 82.

### Example 303

3-{[2′-Fluoro-4′-(trifluoromethoxy)biphenyl-4-yl]methyl}-5-pyridin-3-yl-1,3-oxazolidin-2-one was prepared from 3-(4-bromobenzyl)-5-pyridin-3-yl-1,3-oxazolidin-2-one and 2-fluoro-4-trifluoromethoxyphenylboronic acid using the procedure described for example 82.

### Example 304

### 3-[(2′,4′-difluorobiphenyl-4-yl)methyl]-5-{[ethyl(methyl)amino]methyl}-1,3-oxazolidin-2-one

A solution of 5-(chloromethyl)-3-[(2′,4′-difluorobiphenyl-4-yl)methyl]-1,3-oxazolidin-2-one and ethylmethylamine in DMF (1 mL) was heated in a microwave at 150° C. for 20 min. The mixture was then cooled to room temperature, diluted with 1N HCl (until pH<3) and extracted with ethyl acetate. The aqueous layer was treated with 1N sodium hydroxide until basic (pH ˜9), and was then extracted with ethyl acetate (10 mL×2) and the combined organics were washed with brine, dried over sodium sulfate and concentrated under reduced pressure to give 10 mg of the title compound as an amorphous solid.

### Example 305

### (5R)-3-{4-[(2,4-Difluorophenoxy)methyl]benzyl}-5-methyl-1,3-oxazolidin-2-one

A mixture of (5R)-3-[4-(hydroxymethyl)benzyl]-5-methyl-1,3-oxazolidin-2-one (50 mg, 0.23 mmol), 2,4-difluorophenol (32 mg, 0.25 mmol), triphenylphosphine (65 mg, 0.25 mmol) and di-tert-butylazodicarboxylate (57 mg, 0.25 mmol) in 5 mL of dichloromethane was stirred at room temperature for 18 h. The reaction mixture was then diluted with 10 mL of water, extracted with ethyl acetate and the combined organics were washed with brine, dried over sodium sulfate and concentrated under reduced pressure. Purification by thin layer chromatography on a silica gel plate eluting with 2:3 ethyl acetate/heptane provided 14 mg of the title compound as a colorless oil.

### Example 306

4-(1-Cyclobutylethyl)-1-[(2′,4′-difluorobiphenyl-4-yl)methyl]piperazin-2-one was prepared from 1-[(2′,4′-difluorobiphenyl-4-yl)methyl]piperazin-2-one hydrochloride and cyclobutylmethyl ketone using the procedure described in example 188.

### Example 307

3-[(2′,4′-difluorobiphenyl-4-yl)methyl]-5-[(dimethylamino)methyl]-1,3-oxazolidin-2-one was prepared from 5-(chloromethyl)-3-[(2′,4′-difluorobiphenyl-4-yl)methyl]-1,3-oxazolidin-2-one and dimethylamine using the procedure described in example 305.

Example 308-316 were prepared from either (5R)-3-(4-bromobenzyl)-5-methyl-1,3-oxazolidin-2-one, 3-(4-bromobenzyl)-5,5-dimethyl-1,3-oxazolidin-2-one, 3-(4-bromobenzyl)-5-ethyl-1,3-oxazolidin-2-one or (5R)-3-[(6-chloropyridin-3-yl)methyl]-5-methyl-1,3-oxazolidin-2-one and the appropriate arylboronic add using the procedure described in example 82. Purification of these examples was performed using preparative TLC.

### Examples 317

(5R)-5-methyl-3-{4-[(E)-2-phenylvinyl]benzyl}-1,3-oxazolidin-2-one was prepared from the reaction of (R)-(−)-1-amino-2-propanol with the 4-[(E)-2-phenylvinyl]benzaldehyde using the same procedure as described in example 65.

### Example 318

### (5R)-5-methyl-3-[4-(2-phenylethyl)benzyl]-1,3-oxazolidin-2-one

A solution of (5R)-5-methyl-3-{4-[(E)-2-phenylvinyl]benzyl}-1,3-oxazolidin-2-one (130 mg, 0.44 mmol) and 10% palladium on carbon (20 mg) in ethanol (5 mL) was shaken in a Parr shaker under 40 p.s.i. of hydrogen for 16 h. The flask was purged with nitrogen, filtered through celite and concentrated under reduced pressure to give 120 mg of the title compound as a clear oil.

Intermediate 1

### 4′-Isopropoxybiphenyl-4-carbaldehyde

A mixture of 4-bromobenzaldehyde (1.5 g, 8.1 mmol), 4-isopropoxyphenylboronic acid (1.5 g, 8.1 mmol), tetrakis(triphenylphosphine)palladium(0) (940 mg, 0.8 mmol) and potassium carbonate (2.2 g, 16.2 mmol) in 20 mL of a 1:1 mixture of ethanol and water was stirred at 90° C. After 18 h the mixture was diluted with 25 mL of water and extracted 3 times into ethyl acetate. The combined organics were washed with brine and concentrated under reduced pressure. Purification by silica gel chromatography eluting with 15% ethyl acetate in hexanes have 1.1 g of the title compound as an off-white solid. MS m/z 241.2.

The following biphenyl aldehyde intermediates were prepared by the reaction of 4-bromobenzaldehyde with the appropriate boronic acid using the procedure described for intermediate 1:

| Intermediate # | Intermediate name | Boronic acid | MS m/z |
|---|---|---|---|
| 2 | 4′-ethoxybiphenyl-4- | 4-ethoxyphenyl | 227.3 |
| | carbaldehyde | | |
| 3 | 4′-fluorobiphenyl-4- | 4-fluorophenyl | 241.2 |
| | carbaldehyde | | |
| 4 | 4′-trifluoromethyl- | 4-trifluoromethyl- | [M+] not |
| | biphenyl-4-carbaldehyde | phenyl | observed |

Intermediate 5

### 4-(1-Cyclohexylethoxy)benzaldehyde

A mixture of 4-hydroxybenzaldehyde (500 mg, 4.1 mmol), 1-cyclohexylethanol (0.62 mL, 4.5 mmol), di-tert-butyl azodicarboxylate (1 g, 4.5 mmol) and triphenylphosphine (1.2 g, 4.5 mmol) in 20 mL of anhydrous THF was stirred at room temperature under nitrogen. After 16 h water was added (50 mL) and the resulting mixture was extracted 3× into ethyl acetate. The combined organics were washed with water followed by brine and concentrated under reduced pressure. Purification by silica gel chromatography eluting with 9:1 hexanes/ethyl acetate provided 410 mg of the title compound as a colorless oil. MS m/z 233.1.

The following 4-alkoxybenzaldehyde intermediates were prepared from 4-hydroxybenzaldehyde and the appropriate alcohol using the procedure described for intermediate 2:

| | | | MS |
|---|---|---|---|
| Intermediate # | Intermediate name | alcohol reagent | m/z |
| 6 | 4-[(1R)-1- | (S)-(−)-1- | 227.3 |
| | phenylethoxy]benzaldehyde | phenylethanol | |
| 7 | 4-[(1S)-1- | (R)-(+)-1- | 227.3 |
| | phenylethoxy]benzaldehyde | phenylethanol | |
| 8 | 4- | cyclohexylmethanol | 219.3 |
| | (cyclohexylmethoxy)benzaldehyde | | |
| 9 | 4-(3-phenylpropoxy)benzaldehyde | 3-phenylpropanol | 241.4 |
| 10 | 4-(2-methyl-5-fluorobenzyloxy)benzaldehyde | 2-methyl-5- | 245.3 |
| | | fluorobenzyl | |
| | | alcohol | |
| 11 | 4-(3-(4-fluorophenoxy)propoxy)benzaldehyde | 3-(4-fluorophenoxy)propanol | 275.3 |

Intermediate 12

### (5R)-3-(4-Bromobenzyl)-5-methyl-1,3-oxazolidin-2-one

A mixture of 4-bromobenzaldehyde (3.0 g, 16.2 mmol) and (R)-(−)-1-amino-2-propanol (1.4 mL, 17.8 mmol) in 75 mL of dichloromethane was stirred at room temperature. After 10 min sodium triacetoxyborohydride (12 g, 56.8 mmol) was added at once. After stirring for 6 h at room temperature, 10% ammonium hydroxide (100 mL) as added and the mixture was extracted 3× into dichloromethane. The combined organics were washed with 10% ammonium hydroxide, water and brine, dried over magnesium sulfate and concentrated under reduced pressure to provide 3.8 g of a white solid (MS m/z 244.1 and 246). The solid was dissolved in 75 mL of anhydrous THF and carbonyl diimidazole was added at once. After stirring at 80° C. for 4 h the mixture was cooled to room temperature, diluted with saturated ammonium chloride and extracted 3× into ethyl acetate. The combined organics were washed with water and brine, dried over magnesium sulfate and concentrated under reduced pressure. Purification by chromatography on a silica gel column eluting with 1:9 ethanol/heptane provided 2.8 g of the title compound as a white solid. ¹H NMR (CDCl₃) 400 MHz [symbol] 1.39 (d, 3H), 2.96 (dd, 1H), 3.48 (t, 1H), 4.36 (dd, 2H), 4.62 (m, 1H), 7.16 (d, 2H), 7.48 (d, 2H). MS m/z 270.1 and 272.

Intermediate 13

(5R)-3-[(6-chloropyridin-3-yl)methyl]-5-methyl-1,3-oxazolidin-2-one was prepared from 6-chloro-3-pyridinecarboxaldehyde and (R)-(−)-1-amino-2-propanol using the same procedure as described for intermediate 12.

Intermediate 14

### 1-(4-(1-Cyclohexylethoxy)benzyl)piperazin-2-one hydrochloride

### Step 1

### (4-(1-Cyclohexylethoxy)phenyl)methanol

A solution of 4-(1-cyclohexylethoxy)benzaldehyde (1.538 g) in 70 mL of THF was cooled to 0° C. under nitrogen. Sodium borohydride (252 mg) was slowly added and the mixture was then allowed to warm to room temperature. After stirring 16 h the mixture was quenched by the addition of 3 mL of water and the mixture was then concentrated under reduced pressure. Purification by chromatography on silica gel eluting with a gradient of 0% to 15% ethyl acetate/hexanes provided 1.14 g of (4-(1-cyclohexylethoxy)phenyl)methanol. 400 MHz ¹H NMR (CDCl₃) δ 7.2 (d, 2H), 6.8 (d, 2H), 4.6 (s, 2H), 4.1 (m, 1H), 1.9 (d, 1H), 1.7 (d, 3H), 1.6 (m, 3H), 1.2 (m, 8H).

### Step 2

### 1-Bromo-4-(1-cyclohexylethoxy)benzene

A solution of (4-(1-cyclohexylethoxy)phenyl)methanol (1.14 g) in 20 mL of dichloromethane at 0° C. under nitrogen was treated with a slow addition of 1 M PBr₃ (4.9 mL) in dichloromethane. After stirring 16 h at room temperature the mixture was washed with saturated sodium bicarbonate solution, extracted 2× with dichloromethane, dried over Na₂SO₄, filtered, and the solvents were removed in vacuo to yield 1.36 g of 1-bromo-4-(1-cyclohexylethoxy)benzene.

### Step 3

### tert-Butyl 4-(4-(1-cyclohexylethoxy)benzyl)-3-oxopiperazine-1-carboxylate

A stirred mixture of 1-bromo-4-(1-cyclohexylethoxy)benzene (200 mg) and tert-butyl 3-oxopiperazine-1-carboxylate (134.7 mg) in 1 mL of THF under N₂ at 0° C. was treated dropwise with a mixture of NaH (27 mg) in 2.5 mL of THF. After stirring for 16 h at room temperature the mixture was concentrated and was purified using silica gel chromatography to yield 183 mg of tert-butyl 4-(4-(1-cyclohexylethoxy)benzyl)-3-oxopiperazine-1-carboxylate. MS (M+1) 417.2.

### Step 4

### 1-(4-(1-Cyclohexylethoxy)benzyl)piperazin-2-one hydrochloride

tert-Butyl 4-(4-(1-cyclohexylethoxy)benzyl)-3-oxopiperazine-1-carboxylate (183 mg), 2 mL methanol and 2.5 mL of 4N HCl in dioxane were mixed together and stirred at room temperature for one hour. The mixture was concentrated to yield 155 mg of the title compound. MS (M+1) 317.2.

Intermediate 15

(5R)-3-(4-{[tert-butyl(dimethyl)silyl]oxy}-3-fluorobenzyl)-5-methyl-1,3-oxazolidin-2-one was prepared from 4-{[tert-butyl(dimethyl)silyl]oxy}3-fluorobenzaldehyde and (R)-(−)-1-amino-2-propanol using the same procedure as described for intermediate 12.

Intermediate 16

### 4-{[tert-Butyl(dimethyl)silyl]oxy}-3-fluorobenzaldehyde

A solution of 3-fluoro-4-hydroxybenzaldehyde (500 mg, 3.56 mmol) in dichloromethane at room temperature was treated with tert-butyl(dimethyl)silyl chloride (1.075 g, 7.1 mmol) and triethylamine (720 mg, 7.1 mmol). After stirring 16 h the mixture was diluted with saturated ammonium chloride (50 mL) and the organic layer was washed with brine, dried over sodium sulfate and concentrated under reduced pressure. Purification by chromatography on a silica gel column eluting with a gradient of ethyl acetate and hexane provided 670 mg of the title compound as a transparent oil. MS m/z 255.3.

Intermediate 17

(5R)-3-(4-Bromo-2-fluorobenzyl)-5-methyl-1,3-oxazolidin-2-one was prepared from 4-bromo-2-fluorobenzaldehyde and (R)-(−)-1-amino-2-propanol using the same procedure as described for intermediate 12.

Intermediate 18

(5R)-3-(4{[tert-Butyl(dimethyl)silyl]oxy}-3-methylbenzyl)-5-methyl-1,3-oxazolidin-2-one was prepared from 4-{[tert-butyl(dimethyl)silyl]oxy}-3-methylbenzaldehyde and (R)-(−)-1-amino-2-propanol using the same procedure as described for intermediate 12.

Intermediate 19

4-{[tert-Butyl(dimethyl)silyl]oxy}-3-methylbenzaldehyde was prepared from 3-methyl-4-hydroxybenzaldehyde using the procedure described for intermediate 16.

Intermediate 20

### 2-Fluoro-4-{[(5R)-5-methyl-2-oxo-1,3-oxazolidin-3-yl]methyl}phenyl trifluoromethanesulfonate

To a stirred solution of (5R)-3-(3-fluoro-4-hydroxybenzyl)-5-methyl-1,3-oxazolidin-2-one (35 mg, 0.15 mmol) in 2 mL of THF at 0° C. was added diisopropylamine (17 mg, 0.17 mmol). After 45 min 1,1,1-trifluoro-N-phenyl-N—[(trifluoromethyl)sulfonyl]methanesulfonamide (60 mg, 0.17 mmol) was added. After 16 h at room temperature the mixture was concentrated under reduced pressure. No purification was performed.

Intermediate 21

### (5R)-5-Methyl-3-{4-[(4-oxocyclohexyl)methoxy]benzyl}-1,3-oxazolidin-2-one

To a stirred solution of (5R)-3-[4-(1,4-dioxaspiro[4.5]dec-8-ylmethoxy)benzyl]-5-methyl-1,3-oxazolidin-2-one (40 mg) in acetone was added 4 drops of concentrated sulfuric acid. After heating under reflux for 45 min the mixture was concentrated under reduced pressure, water was added and the resulting aqueous mixture was extracted twice with 50 mL of ethyl acetate. The combined organics were washed with brined, dried over sodium sulfate and concentrated under reduced pressure to provide 30 mg of the title compound as a transparent oil with a reddish tinge.

Intermediate 22

### (5R)-3-[4-(1,4-Dioxaspiro[4.5]dec-8-ylmethoxy)benzyl]-5-methyl-1,3-oxazolidin-2-one

To a solution of (5R)-3-(4-hydroxybenzyl)-5-methyl-1,3-oxazolidin-2-one (100 mg, 0.48 mmol) and 1,4-dioxaspiro[4.5]dec-8-ylmethanol (166 mg, 0.96 mmol) in dichloromethane (5 mL) at room temperature was added polymer bound triphenylphosphine (440 mg of 2.15 mmol/g, 0.96 mmol) followed by di-tert-butyl azodicarboxylate (220 mg, 0.96 mmol). After stirring for 16 h the mixture was diluted with dichloromethane, filtered and concentrated under reduced pressure. Purification by chromatography on a silica gel column eluting with a gradient of 0% to 100% ethyl acetate in heptane provided 20 mg of the title compound as a clear oil. MS m/z 362.4.

Intermediate 23

### (5R)-3-(4-Hydroxybenzyl)-5-methyl-1,3-oxazolidin-2-one

To a stirred solution of (5R)-3-(4-{[tert-butyl(dimethyl)silyl]oxy}benzyl)-5-methyl-1,3-oxazolidin-2-one (50 mg, 0.155 mmol) in 2 mL of THF and 1 mL of DMF at room temperature was added potassium fluoride (10 mg, 0.17 mmol). After stirring at 80° C. for 16 h water was added followed by 1N HCl and the mixture was extracted by ethyl acetate. The combined organics were washed with brine, dried over sodium sulfate, and concentrated under reduced pressure to provide 30 mg of the title compound as a clear oil with an orange tinge.

Intermediate 24

(5R)-3-(4-{[tert-Butyl(dimethyl)silyl]oxy}benzyl)-5-methyl-1,3-oxazolidin-2-one was prepared from 4{[tert-butyl(dimethyl)silyl]oxy}benzaldehyde and (R)-(−)-1-amino-2-propanol using the same procedure as described for intermediate 12.

Intermediate 25

### (5R)-3-{[5-amino-6-(2,3-difluorophenyl)pyridin-3-yl]methyl}-5-methyl-1,3-oxazolidin-2-one

To a solution of (5R)-3-{[6-(2,3-difluorophenyl)-5-nitropyridin-3-yl]methyl}-5-methyl-1,3-oxazolidin-2-one (19 mg) in ethanol in a Parr shaker flask under nitrogen was added 3 mg of palladium (10 wt. % on activated carbon). The reaction mixture was shaken under 40 p.s.i. of hydrogen at room temperature. After 2 h the reaction mixture was purged with nitrogen, filtered through Celite, and concentrated under reduced pressure to give 18 mg of the title compound. MS m/z 320.4.

Intermediate 26

(5R)-3-{[6-(2,3-difluorophenyl)-5-nitropyridin-3-yl]methyl}-5-methyl-1,3-oxazolidin-2-one was prepared from (5R)-3-[(6-chloro-5-nitropyridin-3-yl)methyl]-5-methyl-1,3-oxazolidin-2-one and 2,3-difluorophenylboronic acid using the procedure described in example 82.

Intermediate 27

(5R)-3-[(6-chloro-5-nitropyridin-3-yl)methyl]-5-methyl-1,3-oxazolidin-2-one was prepared from 6-chloro-5-nitronicotinaldehyde and (R)-(−)-1-amino-2-propanol using the procedure described for intermediate 12.

Intermediate 28

2-Methyl-4-{[(5R)-5-methyl-2-oxo-1,3-oxazolidin-3-yl]methyl}phenyl trifluoromethanesulfonate was prepared from (5R)-3-(4-hydroxy-3-methylbenzyl)-5-methyl-1,3-oxazolidin-2-one using the procedure described for intermediate 20.

Intermediate 29

### 4-[(5-Chloro-2,3-dihydro-1H-indol-1-yl)methyl]benzaldehyde

To a stirred solution of DMSO (0.125 mL, 1.75 mmol) in dichloromethane (10 mL) at −70° C. was added oxalyl chloride (133 mg, 0.09 mL, 1.05 mmol) dropwise. After 30 min a solution of {4-[(2,3-dihydro-1H-indol-1-yl)methyl]phenyl}methanol (210 mg, 0.88 mmol) in 10 mL of dichloromethane was slowly added. The mixture was stirred at −70° C. for 30 min, −30° C. for 15 min and then cooled back down to −70° C. before adding triethylamine (0.6 mL, 4.2 mmol). The reaction mixture was warmed to room temperature, diluted with saturated sodium bicarbonate and extracted into dichloromethane. The combined organics were washed with brine, dried over magnesium sulfate and concentrated under reduced pressure. Purification by chromatography on a silica gel column eluting with 9:1 heptane/ethyl acetate provided 130 mg of the title compound as a yellow amorphous solid. MS m/z 272.3.

Intermediate 30

### {4-[(2,3-Dihydro-1H-indol-1-yl)methyl]phenyl}methanol

To a stirred solution of methyl 4-(2,3-dihydro-1H-indol-1-ylmethyl)benzoate (570 mg, 2.1 mmol) in THF (20 mL) at −78° C. was added diisobutylaluminum hydride (2.1 mL of a 1.5 M solution in toluene, 3.2 mmol) slowly over 5 min while keeping the reaction temperature below −65° C. After 1 h at −78° C. the mixture was warmed to room temperature, diluted with saturated ammonium chloride and extracted 3× into ethyl acetate. The combined organics were washed with brine, dried over sodium sulfate and concentrated under reduced pressure to give 210 mg of the title compound as a yellow oil. MS m/z 240.4.

Intermediate 31

### Methyl 4-(2,3-dihydro-1H-indol-1-ylmethyl)benzoate

A mixture of methyl 4-formylbenzoate (350 mg, 2.1 mmol), indoline (280 mg, 2.3 mmol) and sodium triacetoxyborohydride (1.6 g, 7.5 mmol) in 10 mL of dichloromethane was stirred at room temperature for 3 h. The mixture was diluted with 50% aqueous ammonium hydroxide, extracted 3× into dichloromethane. The combined organics were washed with water followed by brine, dried over magnesium sulfate and concentrated under reduced pressure to give 570 mg of the title compound as a colorless oil. MS m/z 268.3.

Intermediate 32

### 4-{[(5R)-5-Methyl-2-oxo-1,3-oxazolidin-3-yl]methyl} benzaldehyde

To a stirred solution of (5R)-3-[4-(1,3-dioxolan-2-yl)benzyl]-5-methyl-1,3-oxazolidin-2-one (880 mg, 3.3 mmol) in 30 mL of acetone was added 1 drop of concentrated sulfuric acid. After 90 min the mixture was cooled to room temperature, diluted with ethyl acetate (100 mL), washed with saturated sodium bicarbonate followed by brine and the organic layer was concentrated under reduced pressure. Purification by chromatography on a silica gel column eluting with 1:1 heptane/ethyl acetate provided 230 mg of the title compound as a colorless oil. MS m/z 220.3.

Intermediate 33

(5R)-3-[4-(1,3-Dioxolan-2-yl)benzyl]-5-methyl-1,3-oxazolidin-2-one was prepared from 4-(1,3-dioxolan-2-yl)benzaldehyde and (R)-(−)-1-amino-2-propanol using the procedure described for intermediate 12.

Intermediate 34

3-(4-Bromobenzyl)-5-pyridin-3-yl-1,3-oxazolidin-2-one was prepared from 4-bromo benzaldehyde and 2-amino-1-(3-pyridyl)ethanol using the procedure described for intermediate 12. MS m/z 333.3.

Intermediate 35

5-(Chloromethyl)-3-[(2′,4′-difluorobiphenyl-4-yl)methyl]-1,3-oxazolidin-2-one was prepared from 3-(4-bromobenzyl)-5-(chloromethyl)-1,3-oxazolidin-2-one and 2,4-difluorophenylboronic acid using the procedure described for example 82.

Intermediate 36

### 3-(4-Bromobenzyl)-5-(chloromethyl)-1,3-oxazolidin-2-one

To a stirred suspension of 1-bromo-4-(chloromethyl)benzene (227 mg, 1.11 mmol) and 5-(chloromethyl)-1,3-oxazolidin-2-one (100 mg, 0.73 mmol) in 2 mL of THF was added sodium hydride (35 mg of 60% in mineral oil, 0.87 mmol) and 1 mL of DMF. The resulting colloid was heated in a microwave oven at 150° C. for 20 min. After cooling to room temperature a white precipitate formed and the mixture was diluted with water and extracted with 100 mL of ethyl acetate. The organic layer was washed with brine, dried over sodium sulfate and concentrated under reduced pressure. Purification by chromatography on a silica gel column eluting with a gradient of 0% to 100% of a 19:1 mixture of ethyl acetate/methanol in heptane provided 120 mg of the title compound as a clear oil. MS m/z 304.2, 306.2, 308.2.

Intermediate 37

### (5R)-3-[4-(Hydroxymethyl)benzyl]-5-methyl-1,3-oxazolidin-2-one

A mixture of methyl 4-{[(5R)-5-methyl-2-oxo-1,3-oxazolidin-3-yl]methyl}benzoate (500 mg, 2.0 mmol), lithium borohydride (65 mg, 3.0 mmol) and methanol (0.12 mL, 3.0 mmol) in 20 mL of ether was stirred at 30° C. After 18 h the reaction mixture was diluted with 100 ml of ethyl acetate, washed with water followed by brine, dried over sodium sulfate and concentrated to provide 360 mg of the title compound as a colorless oil. MS m/z 222.3.

Intermediate 38

Methyl 4-{[(5R)-5-methyl-2-oxo-1,3-oxazolidin-3-yl]methyl}benzoate was prepared from methyl 4-formylbenzoate and (R)-(−)-1-amino-2-propanol using the procedure described for intermediate 12. MS m/z 250.3.

Table 1 shows examples of compounds of the invention having a geometric mean EC₅₀ of less than about 15 micromolar. Table 1A shows NMR data for examples of compounds of Table 1.

TABLE 1

| | mGluR | | | | | | |
|---|---|---|---|---|---|---|---|
| | 2 EC50 | | | Parent | Mass | | |
| | (geometric | | | Mol | Spec | Retention | HPLC |
| Ex | mean) | | IUPACNAME | Weight | (m/z) | time | Method |
| 1 | 761 | nM | (4aR,8aS)-1-[4- | 329.44 | 330.21 | 3.40 | A |
| | | | (cyclobutylmethoxy)benzyl]octahydro- | | | | |
| | | | 2H- | | | | |
| | | | 3,1-benzoxazin-2- | | | | |
| | | | one | | | | |
| 2 | 637 | nM | (4aS,8aR)-1-(4- | 317.43 | 318.19 | 3.37 | A |
| | | | isobutoxybenzyl)octahydro- | | | | |
| | | | 2H-3,1- | | | | |
| | | | benzoxazin-2-one | | | | |
| 3 | 361 | nM | 5-phenyl-3-(4- | 311.38 | 312.17 | 3.28 | A |
| | | | propoxybenzyl)-1,3- | | | | |
| | | | oxazolidin-2-one | | | | |
| 4 | 647 | nM | (4aR,8aS)-1-[4- | 351.44 | 352.17 | 3.26 | A |
| | | | (benzyloxy)benzyl]octahydro- | | | | |
| | | | 2H-3,1- | | | | |
| | | | benzoxazin-2-one | | | | |
| 5 | 1280 | nM | 5-phenyl-3-[4- | 360.41 | 361.15 | 2.26 | A |
| | | | (pyridin-2- | | | | |
| | | | ylmethoxy)benzyl]- | | | | |
| | | | 1,3-oxazolidin-2-one | | | | |
| 6 | 151 | nM | 3-(4- | 325.41 | 326.2 | 3.45 | A |
| | | | isobutoxybenzyl)-5- | | | | |
| | | | phenyl-1,3- | | | | |
| | | | oxazolidin-2-one | | | | |
| 7 | 220 | nM | 3-[4- | 365.47 | 366.18 | 3.75 | A |
| | | | (cyclohexylmethoxy)benzyl]- | | | | |
| | | | 5-phenyl-1,3- | | | | |
| | | | oxazolidin-2-one | | | | |
| 8 | 153 | nM | 3-[4- | 359.42 | 360.13 | 3.35 | A |
| | | | (benzyloxy)benzyl]-5- | | | | |
| | | | phenyl-1,3- | | | | |
| | | | oxazolidin-2-one | | | | |
| 9 | 135 | nM | 3-[4- | 337.42 | 338.17 | 3.48 | A |
| | | | (cyclobutylmethoxy)benzyl]- | | | | |
| | | | 5-phenyl-1,3- | | | | |
| | | | oxazolidin-2-one | | | | |
| 10 | 395 | nM | 3-[4-(2- | 379.5 | 380.18 | 3.85 | A |
| | | | cyclohexylethoxy)benzyl]- | | | | |
| | | | 5-phenyl-1,3- | | | | |
| | | | oxazolidin-2-one | | | | |
| 11 | 699 | nM | 3-[4-(2- | 303.4 | 304.19 | 3.52 | A |
| | | | cyclohexylethoxy)benzyl]- | | | | |
| | | | 1,3-oxazolidin-2- | | | | |
| | | | one | | | | |
| 12 | 590 | nM | 3-[4- | 289.37 | 290.18 | 3.38 | A |
| | | | (cyclohexylmethoxy)benzyl]- | | | | |
| | | | 1,3- | | | | |
| | | | oxazolidin-2-one | | | | |
| 13 | 218 | nM | 5-phenyl-3-[4- | 360.41 | 361.15 | 2.19 | A |
| | | | (pyridin-3- | | | | |
| | | | ylmethoxy)benzyl]- | | | | |
| | | | 1,3-oxazolidin-2-one | | | | |
| 14 | 178 | nM | 3-[4- | 275.35 | 276.18 | 3.17 | A |
| | | | (cyclobutylmethoxy)benzyl]- | | | | |
| | | | 5-methyl-1,3- | | | | |
| | | | oxazolidin-2-one | | | | |
| 15 | 935 | nM | 3-[4- | 303.4 | 304.19 | 3.26 | A |
| | | | (cyclobutylmethoxy)benzyl]- | | | | |
| | | | 5,5-dimethyl- | | | | |
| | | | 1,3-oxazinan-2-one | | | | |
| 16 | 663 | nM | 3-(4- | 291.39 | 292.21 | 3.23 | A |
| | | | isobutoxybenzyl)-5,5- | | | | |
| | | | dimethyl-1,3- | | | | |
| | | | oxazinan-2-one | | | | |
| 17 | 696 | nM | 3-[4- | 331.45 | 332.25 | 3.58 | A |
| | | | (cyclohexylmethoxy)benzyl]- | | | | |
| | | | 5,5-dimethyl- | | | | |
| | | | 1,3-oxazinan-2-one | | | | |
| 18 | <15.0 | nM | 3-[4- | 303.4 | 304.18 | 3.50 | A |
| | | | (cyclohexylmethoxy)benzyl]- | | | | |
| | | | 5-methyl-1,3- | | | | |
| | | | oxazolidin-2-one | | | | |
| 19 | 203 | nM | 3-[4-(2- | 317.43 | 318.22 | 3.63 | A |
| | | | cyclohexylethoxy)benzyl]- | | | | |
| | | | 5-methyl-1,3- | | | | |
| | | | oxazolidin-2-one | | | | |
| 20 | 979 | nM | 3-(4- | 263.34 | 264.21 | 3.14 | A |
| | | | isobutoxybenzyl)-5- | | | | |
| | | | methyl-1,3- | | | | |
| | | | oxazolidin-2-one | | | | |
| 21 | 819 | nM | 3-[4- | 325.41 | 326.20 | 3.13 | A |
| | | | (benzyloxy)benzyl]- | | | | |
| | | | 5,5-dimethyl-1,3- | | | | |
| | | | oxazinan-2-one | | | | |
| 22 | 262 | nM | 3-[4- | 297.35 | 298.15 | 3.05 | A |
| | | | (benzyloxy)benzyl]-5- | | | | |
| | | | methyl-1,3- | | | | |
| | | | oxazolidin-2-one | | | | |
| 23 | 887 | nM | (4S,5R)-4-methyl-5- | 325.41 | 326.20 | 3.40 | A |
| | | | phenyl-3-(4- | | | | |
| | | | propoxybenzyl)-1,3- | | | | |
| | | | oxazolidin-2-one | | | | |
| 24 | 660 | nM | (4S,5R)-3-[4- | 351.44 | 352.17 | 3.59 | A |
| | | | (cyclobutylmethoxy)benzyl]- | | | | |
| | | | 4-methyl-5- | | | | |
| | | | phenyl-1,3- | | | | |
| | | | oxazolidin-2-one | | | | |
| 25 | 1760 | nM | (4S,5R)-4-methyl-5- | 374.44 | 375.15 | 2.36 | A |
| | | | phenyl-3-[4-(pyridin- | | | | |
| | | | 2-ylmethoxy)benzyl]- | | | | |
| | | | 1,3-oxazolidin-2-one | | | | |
| 26 | 542 | nM | (4S,5R)-3-(4- | 339.43 | 340.20 | 3.56 | A |
| | | | isobutoxybenzyl)-4- | | | | |
| | | | methyl-5-phenyl-1,3- | | | | |
| | | | oxazolidin-2-one | | | | |
| 27 | 1140 | nM | (4S,5R)-3-[4- | 379.5 | 380.18 | 3.85 | A |
| | | | (cyclohexylmethoxy)benzyl]- | | | | |
| | | | 4-methyl-5- | | | | |
| | | | phenyl-1,3- | | | | |
| | | | oxazolidin-2-one | | | | |
| 28 | 1240 | nM | 3-[4- | 275.35 | 276.17 | 2.97 | A |
| | | | (cyclobutylmethoxy)benzyl]- | | | | |
| | | | 1,3-oxazinan- | | | | |
| | | | 2-one | | | | |
| 29 | 467 | nM | (4S,5R)-3-[4- | 373.45 | 374.15 | 3.45 | A |
| | | | (benzyloxy)benzyl]-4- | | | | |
| | | | methyl-5-phenyl-1,3- | | | | |
| | | | oxazolidin-2-one | | | | |
| 30 | 987 | nM | (4S,5R)-3-[4-(2- | 393.52 | 394.23 | 3.94 | A |
| | | | cyclohexylethoxy)benzyl]- | | | | |
| | | | 4-methyl-5- | | | | |
| | | | phenyl-1,3- | | | | |
| | | | oxazolidin-2-one | | | | |
| 31 | 1250 | nM | 3-[4- | 297.35 | 298.13 | 2.88 | A |
| | | | (benzyloxy)benzyl]- | | | | |
| | | | 1,3-oxazinan-2-one | | | | |
| 32 | 245 | nM | (4S,5R)-4-methyl-5- | 374.44 | 375.15 | 2.28 | A |
| | | | phenyl-3-[4-(pyridin- | | | | |
| | | | 3-ylmethoxy)benzyl]- | | | | |
| | | | 1,3-oxazolidin-2-one | | | | |
| 33 | 117 | nM | 3-[4- | 303.4 | 304.20 | 3.32 | A |
| | | | (cyclohexylmethoxy)benzyl]- | | | | |
| | | | 1,3-oxazinan- | | | | |
| | | | 2-one | | | | |
| 34 | 282 | nM | 3-[4-(2- | 317.43 | 318.23 | 3.45 | A |
| | | | cyclohexylethoxy)benzyl]- | | | | |
| | | | 1,3-oxazinan-2- | | | | |
| | | | one | | | | |
| 35 | 884 | nM | (4aR,8aS)-1-[4-(1- | 365.47 | 366.18 | 2.94 | B |
| | | | phenylethoxy)benzyl]octahydro- | | | | |
| | | | 2H-3,1- | | | | |
| | | | benzoxazin-2-one | | | | |
| 36 | 571 | nM | 5-phenyl-3-[4- | 353.42 | 354.15 | 2.38 | B |
| | | | (tetrahydro-2H- | | | | |
| | | | pyran-4- | | | | |
| | | | yloxy)benzyl]-1,3- | | | | |
| | | | oxazolidin-2-one | | | | |
| 37 | 2020 | nM | (4aR,8aS)-1-[4-(1- | 371.52 | 372.22 | 3.42 | B |
| | | | cyclohexylethoxy)benzyl]octahydro- | | | | |
| | | | 2H-3,1- | | | | |
| | | | benzoxazin-2-one | | | | |
| 38 | 127 | nM | 3-[4-(1- | 379.5 | 380.18 | 2.38 | B |
| | | | cyclohexylethoxy)benzyl]- | | | | |
| | | | 5-phenyl-1,3- | | | | |
| | | | oxazolidin-2-one | | | | |
| 39 | 244 | nM | 5-phenyl-3-[4-(1- | 373.45 | 374.13 | 3.07 | B |
| | | | phenylethoxy)benzyl]- | | | | |
| | | | 1,3-oxazolidin-2-one | | | | |
| 40 | 155 | nM | 3-[4- | 351.44 | 352.17 | 3.17 | B |
| | | | (cyclohexyloxy)benzyl]- | | | | |
| | | | 5-phenyl-1,3- | | | | |
| | | | oxazolidin-2-one | | | | |
| 41 | 386 | nM | (4aR,8aS)-1-[4- | 343.46 | 344.20 | 3.04 | B |
| | | | (cyclohexyloxy)benzyl]octahydro- | | | | |
| | | | 2H-3,1- | | | | |
| | | | benzoxazin-2-one | | | | |
| 42 | 677 | nM | 3-[4-(1- | 303.4 | 304.18 | 3.00 | B |
| | | | cyclohexylethoxy)benzyl]- | | | | |
| | | | 1,3-oxazolidin-2- | | | | |
| | | | one | | | | |
| 43 | 336 | nM | 3-[4- | 317.43 | 318.18 | 2.86 | B |
| | | | (cyclohexyloxy)benzyl]- | | | | |
| | | | 5,5-dimethyl-1,3- | | | | |
| | | | oxazinan-2-one | | | | |
| 44 | 572 | nM | 3-(4- | 311.38 | 312.13 | 2.76 | B |
| | | | isopropoxybenzyl)-5- | | | | |
| | | | phenyl-1,3- | | | | |
| | | | oxazolidin-2-one | | | | |
| 45 | 213 | nM | 5,5-dimethyl-3-[4-(1- | 339.43 | 340.15 | 2.78 | B |
| | | | phenylethoxy)benzyl]- | | | | |
| | | | 1,3-oxazinan-2-one | | | | |
| 46 | 151 | nM | 5-methyl-3-[4-(1- | 311.38 | 312.13 | 2.68 | B |
| | | | phenylethoxy)benzyl]- | | | | |
| | | | 1,3-oxazolidin-2-one | | | | |
| 47 | 247 | nM | 3-[4-(1- | 345.48 | 346.19 | 3.26 | B |
| | | | cyclohexylethoxy)benzyl]- | | | | |
| | | | 5,5-dimethyl-1,3- | | | | |
| | | | oxazinan-2-one | | | | |
| 48 | <7.88 | nM | 3-[4-(1- | 317.43 | 318.18 | 3.16 | B |
| | | | cyclohexylethoxy)benzyl]- | | | | |
| | | | 5-methyl-1,3- | | | | |
| | | | oxazolidin-2-one | | | | |
| 49 | 236 | nM | 3-[4- | 289.37 | 290.18 | 2.75 | B |
| | | | (cyclohexyloxy)benzyl]- | | | | |
| | | | 5-methyl-1,3- | | | | |
| | | | oxazolidin-2-one | | | | |
| 50 | 263 | nM | (4S,5R)-4-methyl-5- | 387.48 | 388.14 | 3.21 | B |
| | | | phenyl-3-[4-(1- | | | | |
| | | | phenylethoxy)benzyl]- | | | | |
| | | | 1,3-oxazolidin-2-one | | | | |
| 56 | 356 | nM | (4S,5R)-3-[4- | 365.47 | 366.18 | 3.33 | B |
| | | | (cyclohexyloxy)benzyl]- | | | | |
| | | | 4-methyl-5-phenyl- | | | | |
| | | | 1,3-oxazolidin-2-one | | | | |
| 57 | <40.8 | nM | (5R)-3-[(4′- | 325.41 | 326.1 | 2.8 | E |
| | | | isopropoxybiphenyl- | | | | |
| | | | 4-yl)methyl]-5- | | | | |
| | | | methyl-1,3- | | | | |
| | | | oxazolidin-2-one | | | | |
| 58 | 105 | nM | (5S)-3-[(4′- | 325.41 | 326.1 | 2.8 | E |
| | | | isopropoxybiphenyl- | | | | |
| | | | 4-yl)methyl]-5- | | | | |
| | | | methyl-1,3- | | | | |
| | | | oxazolidin-2-one | | | | |
| 59 | 667 | nM | (5S)-3-[(4′- | 311.38 | 312.1 | 2.7 | E |
| | | | ethoxybiphenyl-4- | | | | |
| | | | yl)methyl]-5-methyl- | | | | |
| | | | 1,3-oxazolidin-2-one | | | | |
| 60 | 97.0 | nM | (5R)-3-[(4′- | 311.38 | 312.1 | 2.8 | E |
| | | | ethoxybiphenyl-4- | | | | |
| | | | yl)methyl]-5-methyl- | | | | |
| | | | 1,3-oxazolidin-2-one | | | | |
| 61 | 84.1 | nM | (5R)-3-[(4′- | 285.32 | 286.1 | 2.5 | E |
| | | | fluorobiphenyl-4- | | | | |
| | | | yl)methyl]-5-methyl- | | | | |
| | | | 1,3-oxazolidin-2-one | | | | |
| 62 | 198 | nM | (5S)-3-[(4′- | 285.32 | 286.1 | 2.5 | E |
| | | | fluorobiphenyl-4- | | | | |
| | | | yl)methyl]-5-methyl- | | | | |
| | | | 1,3-oxazolidin-2-one | | | | |
| 63 | 141 | nM | (5S)-5-methyl-3-{[4′- | 335.32 | 336.1 | 2.8 | E |
| | | | (trifluoromethyl)biphenyl- | | | | |
| | | | 4-yl]methyl}-1,3- | | | | |
| | | | oxazolidin-2-one | | | | |
| 64 | 61.0 | nM | (5R)-5-methyl-3-{[4′- | 335.32 | 336.1 | 2.8 | E |
| | | | (trifluoromethyl)biphenyl- | | | | |
| | | | 4-yl]methyl}-1,3- | | | | |
| | | | oxazolidin-2-one | | | | |
| 65 | 4.96 | nM | (5R)-3-[4-(1- | 317.43 | 318.2 | 3 | E |
| | | | cyclohexylethoxy)benzyl]- | | | | |
| | | | 5-methyl-1,3- | | | | |
| | | | oxazolidin-2-one | | | | |
| 66 | 22.1 | nM | (5S)-3-[4-(1- | 317.43 | 318.2 | 3 | E |
| | | | cyclohexylethoxy)benzyl]- | | | | |
| | | | 5-methyl-1,3- | | | | |
| | | | oxazolidin-2-one | | | | |
| 67 | 240 | nM | (5R)-5-methyl-3-{4- | 311.38 | 312.1 | 2.7 | E |
| | | | [(1R)-1- | | | | |
| | | | phenylethoxy]benzyl}- | | | | |
| | | | 1,3-oxazolidin-2-one | | | | |
| 68 | 299 | nM | (5S)-5-methyl-3-{4- | 311.38 | 312.1 | 2.7 | E |
| | | | [(1R)-1- | | | | |
| | | | phenylethoxy]benzyl}- | | | | |
| | | | 1,3-oxazolidin-2-one | | | | |
| 69 | 173 | nM | (5R)-5-methyl-3-{4- | 311.38 | 312.1 | 2.6 | E |
| | | | [(1S)-1- | | | | |
| | | | phenylethoxy]benzyl}- | | | | |
| | | | 1,3-oxazolidin-2-one | | | | |
| 70 | 209 | nM | (5S)-5-methyl-3-{4- | 311.38 | 312.1 | 2.6 | E |
| | | | [(1S)-1- | | | | |
| | | | phenylethoxy]benzyl}- | | | | |
| | | | 1,3-oxazolidin-2-one | | | | |
| 82 | 142 | nM | (5R)-3-(biphenyl-4- | 267.33 | 268.2 | 2.5 | E |
| | | | ylmethyl)-5-methyl- | | | | |
| | | | 1,3-oxazolidin-2-one | | | | |
| 83 | 28.6 | nM | (5R)-3-[4-(2,3- | 309.36 | 310.1 | 2.5 | E |
| | | | dihydro-1- | | | | |
| | | | benzofuran-5- | | | | |
| | | | yl)benzyl]-5-methyl- | | | | |
| | | | 1,3-oxazolidin-2-one | | | | |
| 83 | 35.3 | nM | (5R)-3-[(2′- | 285.32 | 286.2 | 2.4 | E |
| | | | fluorobiphenyl-4- | | | | |
| | | | yl)methyl]-5-methyl- | | | | |
| | | | 1,3-oxazolidin-2-one | | | | |
| 85 | 498 | nM | 4′-{[(5R)-5-methyl-2- | 292.34 | 293.2 | 2.2 | E |
| | | | oxo-1,3-oxazolidin-3- | | | | |
| | | | yl]methyl}biphenyl-3- | | | | |
| | | | carbonitrile | | | | |
| 86 | 75.8 | nM | (5R)-3-[(4′- | 301.77 | 302.1 | 2.8 | E |
| | | | chlorobiphenyl-4- | | | | |
| | | | yl)methyl]-5-methyl- | | | | |
| | | | 1,3-oxazolidin-2-one | | | | |
| 87 | 1380 | nM | (5R)-3-[(2,2-dimethyl- | 275.35 | 275.3 | 2.5 | E |
| | | | 3,4-dihydro-2H- | | | | |
| | | | chromen-6- | | | | |
| | | | yl)methyl]-5-methyl- | | | | |
| | | | 1,3-oxazolidin-2-one | | | | |
| 88 | 66.6 | nM | 3-{4-[(4-chloro-2- | 349.79 | 350.0741 | 1.3 | C |
| | | | fluorobenzyl)oxy]benzyl}- | | | | |
| | | | 5-methyl-1,3- | | | | |
| | | | oxazolidin-2-one | | | | |
| 89 | 69.2 | nM | 3-{4-[(4- | 331.8 | 332.0349 | 1.28 | C |
| | | | chlorobenzyl)oxy]benzyl}- | | | | |
| | | | 5-methyl-1,3- | | | | |
| | | | oxazolidin-2-one | | | | |
| 90 | 25.5 | nM | 3-[4- | 289.37 | 290.0916 | 1.34 | C |
| | | | (cyclopentylmethoxy)benzyl]- | | | | |
| | | | 5-methyl-1,3- | | | | |
| | | | oxazolidin-2-one | | | | |
| 91 | 152 | nM | 3-{4-[(3,5- | 333.33 | 334.0834 | 1.23 | C |
| | | | difluorobenzyl)oxy]benzyl}- | | | | |
| | | | 5-methyl-1,3- | | | | |
| | | | oxazolidin-2-one | | | | |
| 92 | 10.8 | nM | 3-[4-(2- | 303.4 | 304.1172 | 1.39 | C |
| | | | cyclopentylethoxy)benzyl]- | | | | |
| | | | 5-methyl-1,3- | | | | |
| | | | oxazolidin-2-one | | | | |
| 93 | 187 | nM | 3-[4-(2- | 275.35 | 276.0665 | 1.2 | C |
| | | | cyclopropylethoxy)benzyl]- | | | | |
| | | | 5-methyl-1,3- | | | | |
| | | | oxazolidin-2-one | | | | |
| 94 | 42.0 | nM | 5-methyl-3-{4-[(3- | 291.39 | 292.1049 | 1.39 | C |
| | | | methylpentyl)oxy]benzyl}- | | | | |
| | | | 1,3-oxazolidin-2- | | | | |
| | | | one | | | | |
| 95 | 723 | nM | 5-methyl-3-{4-[(1- | 275.35 | 276.0665 | 1.19 | C |
| | | | methylcyclopropyl)methoxy]benzyl}- | | | | |
| | | | 1,3- | | | | |
| | | | oxazolidin-2-one | | | | |
| 96 | <14.9 | nM | 3-[4-(3,3- | 291.39 | 292.1049 | 1.37 | C |
| | | | dimethylbutoxy)benzyl]- | | | | |
| | | | 5-methyl-1,3- | | | | |
| | | | oxazolidin-2-one | | | | |
| 97 | 823 | nM | 3-{4-[(1-isopropyl-1H- | 379.46 | 380.1859 | 1.07 | C |
| | | | benzimidazol-2- | | | | |
| | | | yl)methoxy]benzyl}-5- | | | | |
| | | | methyl-1,3- | | | | |
| | | | oxazolidin-2-one | | | | |
| 98 | 7.07 | nM | 3-{4-[3-(4- | 355.43 | 356.1599 | 1.29 | C |
| | | | methoxyphenyl)propoxy]benzyl}- | | | | |
| | | | 5-methyl- | | | | |
| | | | 1,3-oxazolidin-2-one | | | | |
| 99 | 115 | nM | 3-{4-[2-(3- | 345.82 | 346.0803 | 1.33 | C |
| | | | chlorophenyl)ethoxy]benzyl}- | | | | |
| | | | 5-methyl-1,3- | | | | |
| | | | oxazolidin-2-one | | | | |
| 100 | 55.9 | nM | 5-methyl-3-(4-{[(2S)- | 277.36 | 278.0926 | 1.31 | C |
| | | | 2- | | | | |
| | | | methylbutyl]oxy}benzyl)- | | | | |
| | | | 1,3-oxazolidin-2- | | | | |
| | | | one | | | | |
| 101 | 188 | nM | 3-{4-[2-(3- | 329.37 | 330.1031 | 1.25 | C |
| | | | fluorophenyl)ethoxy]benzyl}- | | | | |
| | | | 5-methyl-1,3- | | | | |
| | | | oxazolidin-2-one | | | | |
| 102 | 282 | nM | 5-methyl-3-[4-(2- | 311.38 | 312.1031 | 1.24 | C |
| | | | phenylethoxy)benzyl]- | | | | |
| | | | 1,3-oxazolidin-2-one | | | | |
| 103 | 221 | nM | 3-{4-[(3- | 327.38 | 328.0908 | 1.17 | C |
| | | | methoxybenzyl)oxy]benzyl}- | | | | |
| | | | 5-methyl-1,3- | | | | |
| | | | oxazolidin-2-one | | | | |
| 104 | 37.9 | nM | 5-methyl-3-[4-(3- | 325.41 | 326.1291 | 1.32 | C |
| | | | phenylpropoxy)benzyl]- | | | | |
| | | | 1,3-oxazolidin-2- | | | | |
| | | | one | | | | |
| 105 | 217 | nM | 3-{4-[2-(3- | 341.41 | 342.1558 | 1.22 | C |
| | | | methoxyphenyl)ethoxy]benzyl}- | | | | |
| | | | 5-methyl- | | | | |
| | | | 1,3-oxazolidin-2-one | | | | |
| 106 | 73.7 | nM | 3-{4-[(2,6- | 333.33 | 334.0773 | 1.17 | C |
| | | | difluorobenzyl)oxy]benzyl}- | | | | |
| | | | 5-methyl-1,3- | | | | |
| | | | oxazolidin-2-one | | | | |
| 107 | 273 | nM | 5-methyl-3-{4-[2-(5- | 392.45 | 393.1751 | 1.27 | C |
| | | | methyl-2-phenyl-1,3- | | | | |
| | | | oxazol-4- | | | | |
| | | | yl)ethoxy]benzyl}-1,3- | | | | |
| | | | oxazolidin-2-one | | | | |
| 108 | 91.2 | nM | 3-{4-[(3- | 331.8 | 332.0349 | 128 | C |
| | | | chlorobenzyl)oxy]benzyl}- | | | | |
| | | | 5-methyl-1,3- | | | | |
| | | | oxazolidin-2-one | | | | |
| 109 | 219 | nM | 4-(2-{4-[(5-methyl-2- | 336.39 | 337.1187 | 1.14 | C |
| | | | oxo-1,3-oxazolidin-3- | | | | |
| | | | yl)methyl]phenoxy}ethyl)benzonitrile | | | | |
| 110 | 243 | nM | 3-{4-[(4- | 315.34 | 316.0626 | 1.18 | C |
| | | | fluorobenzyl)oxy]benzyl}- | | | | |
| | | | 5-methyl-1,3- | | | | |
| | | | oxazolidin-2-one | | | | |
| 111 | 48.3 | nM | 5-methyl-3-(4-{[(2S)- | 325.41 | 326.1291 | 1.32 | C |
| | | | 2- | | | | |
| | | | phenylpropyl]oxy}benzyl)- | | | | |
| | | | 1,3-oxazolidin-2- | | | | |
| | | | one | | | | |
| 112 | 138 | nM | 3-{4-[(2- | 315.34 | 316.0626 | 1.19 | C |
| | | | fluorobenzyl)oxy]benzyl}- | | | | |
| | | | 5-methyl-1,3- | | | | |
| | | | oxazolidin-2-one | | | | |
| 113 | 244 | nM | 3-{4-[(3,5- | 357.4 | 375.1755 | 1.17 | C |
| | | | dimethoxybenzyl)oxy]benzyl}- | | | | |
| | | | 5-methyl- | | | | |
| | | | 1,3-oxazolidin-2-one | | | | |
| 114 | 25.7 | nM | 3-{4-[(2- | 341.41 | 359.1482 | 1.29 | C |
| | | | ethoxybenzyl)oxy]benzyl}- | | | | |
| | | | 5-methyl-1,3- | | | | |
| | | | oxazolidin-2-one | | | | |
| 115 | 71.8 | nM | 3-{4-[(2-chloro-4- | 349.79 | 350.0695 | 1.3 | C |
| | | | fluorobenzyl)oxy]benzyl}- | | | | |
| | | | 5-methyl-1,3- | | | | |
| | | | oxazolidin-2-one | | | | |
| 116 | 934 | nM | 5-methyl-3-[4-(3- | 326.39 | 327.1358 | 0.95 | C |
| | | | pyridin-4- | | | | |
| | | | ylpropoxy)benzyl]- | | | | |
| | | | 1,3-oxazolidin-2-one | | | | |
| 117 | 34.5 | nM | 3-{4-[(2- | 331.8 | 332.0674 | 1.27 | C |
| | | | chlorobenzyl)oxy]benzyl}- | | | | |
| | | | 5-methyl-1,3- | | | | |
| | | | oxazolidin-2-one | | | | |
| 118 | 89.4 | nM | 3-{4-[(2- | 327.38 | 345.1361 | 1.2 | C |
| | | | methoxybenzyl)oxy]benzyl}- | | | | |
| | | | 5-methyl-1,3- | | | | |
| | | | oxazolidin-2-one | | | | |
| 119 | 164 | nM | 3-{4-[(4- | 341.41 | 359.1665 | 1.25 | C |
| | | | ethoxybenzyl)oxy]benzyl}- | | | | |
| | | | 5-methyl-1,3- | | | | |
| | | | oxazolidin-2-one | | | | |
| 120 | 1150 | nM | 5-methyl-3-[4-(3- | 326.39 | 327.1358 | 0.95 | C |
| | | | pyridin-3- | | | | |
| | | | ylpropoxy)benzyl]- | | | | |
| | | | 1,3-oxazolidin-2-one | | | | |
| 121 | 434 | nM | 5-methyl-3-[4-(2- | 327.38 | 328.1107 | 1.18 | C |
| | | | phenoxyethoxy)benzyl]- | | | | |
| | | | 1,3-oxazolidin-2- | | | | |
| | | | one | | | | |
| 122 | 40.7 | nM | 5-methyl-3-[4-(1- | 325.41 | 326.1291 | 1.29 | C |
| | | | methyl-2- | | | | |
| | | | phenylethoxy)benzyl]- | | | | |
| | | | 1,3-oxazolidin-2-one | | | | |
| 123 | 22.6 | nM | 3-{4-[(1R,2S,4S)- | 301.38 | 302.1039 | 1.34 | C |
| | | | bicyclo[2.2.1]hept-2- | | | | |
| | | | yloxy]benzyl}-5- | | | | |
| | | | methyl-1,3- | | | | |
| | | | oxazolidin-2-one | | | | |
| 124 | 827 | nM | 4-({4-[(5-methyl-2- | 322.36 | 323.1092 | 1.09 | C |
| | | | oxo-1,3-oxazolidin-3- | | | | |
| | | | yl)methyl]phenoxy}methyl)benzonitrile | | | | |
| 125 | 24.1 | nM | 5-methyl-3-{4-[1-(4- | 325.41 | 326.1291 | 1.32 | C |
| | | | methylphenyl)ethoxy]benzyl}- | | | | |
| | | | 1,3- | | | | |
| | | | oxazolidin-2-one | | | | |
| 126 | 83.1 | nM | 3-[4-(2,3-dihydro-1H- | 323.39 | 324.1158 | 1.26 | C |
| | | | inden-2- | | | | |
| | | | yloxy)benzyl]-5- | | | | |
| | | | methyl-1,3- | | | | |
| | | | oxazolidin-2-one | | | | |
| 127 | 1680 | nM | 3-{4-[(4-fluoro-3- | 345.37 | 346.1111 | 1.17 | C |
| | | | methoxybenzyl)oxy]benzyl}- | | | | |
| | | | 5-methyl-1,3- | | | | |
| | | | oxazolidin-2-one | | | | |
| 128 | 595 | nM | 5-methyl-3-{4-[(5- | 377.44 | 378.1849 | 1.1 | C |
| | | | methyl-1-phenyl-1H- | | | | |
| | | | pyrazol-4- | | | | |
| | | | yl)methoxy]benzyl}- | | | | |
| | | | 1,3-oxazolidin-2-one | | | | |
| 129 | 155 | nM | 3-({4-[(5-methyl-2- | 322.36 | 323.1092 | 1.1 | C |
| | | | oxo-1,3-oxazolidin-3- | | | | |
| | | | yl)methyl]phenoxy}methyl)benzonitrile | | | | |
| 130 | 80.1 | nM | 3-{4-[(1R,2R,4S)- | 301.38 | 302.1039 | 1.34 | C |
| | | | bicydo[2.2.1]hept-2- | | | | |
| | | | yloxy]benzyl}-5- | | | | |
| | | | methyl-1,3- | | | | |
| | | | oxazolidin-2-one | | | | |
| 131 | 125 | nM | 5-methyl-3-(4-{[4- | 363.42 | 364.1295 | 1.12 | C |
| | | | (1H-pyrazol-1- | | | | |
| | | | yl)benzyl]oxy}benzyl)- | | | | |
| | | | 1,3-oxazolidin-2-one | | | | |
| 132 | 1670 | nM | 5-methyl-3-{4-[(5- | 378.43 | 379.1615 | 1.18 | C |
| | | | methyl-3- | | | | |
| | | | phenylisoxazol-4- | | | | |
| | | | yl)methoxy]benzyl}- | | | | |
| | | | 1,3-oxazolidin-2-one | | | | |
| 133 | 120 | nM | 5-methyl-3-{4-[(4- | 389.45 | 390.1708 | 1.26 | C |
| | | | methyl-2- | | | | |
| | | | phenylpyrimidin-5- | | | | |
| | | | yl)methoxy]benzyl}- | | | | |
| | | | 1,3-oxazolidin-2-one | | | | |
| 134 | 214 | nM | 5-methyl-3-(4-{[(1S)- | 277.36 | 278.0796 | 1.26 | C |
| | | | 1- | | | | |
| | | | methylbutyl]oxy}benzyl)- | | | | |
| | | | 1,3-oxazolidin-2- | | | | |
| | | | one | | | | |
| 135 | 1890 | nM | 3-[4-(1- | 275.35 | 276.0665 | 1.13 | C |
| | | | cyclopropylethoxy)benzyl]- | | | | |
| | | | 5-methyl-1,3- | | | | |
| | | | oxazolidin-2-one | | | | |
| 136 | 774 | nM | 3-[4-(1- | 277.36 | 278.0796 | 1.26 | C |
| | | | ethylpropoxy)benzyl]- | | | | |
| | | | 5-methyl-1,3- | | | | |
| | | | oxazolidin-2-one | | | | |
| 137 | 310 | nM | 3-{4-[(3,4- | 333.33 | 334.0482 | 1.22 | C |
| | | | difluorobenzyl)oxy]benzyl}- | | | | |
| | | | 5-methyl-1,3- | | | | |
| | | | oxazolidin-2-one | | | | |
| 138 | 87.3 | nM | 3-{4-[(3- | 341.41 | 359.1482 | 1.26 | C |
| | | | ethoxybenzyl)oxy]benzyl}- | | | | |
| | | | 5-methyl-1,3- | | | | |
| | | | oxazolidin-2-one | | | | |
| 139 | 79.7 | nM | 5-methyl-3-{4-[(1R)- | 311.38 | 312.1031 | 1.22 | C |
| | | | 1- | | | | |
| | | | phenylethoxy]benzyl}- | | | | |
| | | | 1,3-oxazolidin-2-one | | | | |
| 140 | 85.8 | nM | 3-[4-(2,3-dihydro-1H- | 323.39 | 324.1158 | 1.26 | C |
| | | | inden-1- | | | | |
| | | | yloxy)benzyl]-5- | | | | |
| | | | methyl-1,3- | | | | |
| | | | oxazolidin-2-one | | | | |
| 141 | 995 | nM | 3-{4-[2- | 382.5 | 383.2421 | 1.36 | C |
| | | | (dimethylamino)-2- | | | | |
| | | | phenylbutoxy]benzyl}- | | | | |
| | | | 5-methyl-1,3- | | | | |
| | | | oxazolidin-2-one | | | | |
| 142 | 43.5 | nM | 3-{4-[(5-fluoro-2- | 329.37 | 330.0886 | 1.26 | C |
| | | | methylbenzyl)oxy]benzyl}- | | | | |
| | | | 5-methyl-1,3- | | | | |
| | | | oxazolidin-2-one | | | | |
| 143 | 75.5 | nM | 5-methyl-3-[4-(3- | 277.36 | 278.0796 | 1.3 | C |
| | | | methylbutoxy)benzyl]- | | | | |
| | | | 1,3-oxazolidin-2-one | | | | |
| 144 | 84.8 | nM | 5-methyl-3-{4-[(1S)- | 311.38 | 312.1031 | 1.22 | C |
| | | | 1- | | | | |
| | | | phenylethoxy]benzyl}- | | | | |
| | | | 1,3-oxazolidin-2-one | | | | |
| 145 | 1340 | nM | 3-(4-{[(2R)-2- | 382.5 | 383.2421 | 1.35 | C |
| | | | (dimethylamino)-2- | | | | |
| | | | phenylbutyl]oxy}benzyl)- | | | | |
| | | | 5-methyl-1,3- | | | | |
| | | | oxazolidin-2-one | | | | |
| 146 | 1110 | nM | 3-{4-[(3-methoxy-4- | 341.41 | 342.1497 | 1.28 | C |
| | | | methylbenzyl)oxy]benzyl}- | | | | |
| | | | 5-methyl-1,3- | | | | |
| | | | oxazolidin-2-one | | | | |
| 147 | 118 | nM | 3-{4-[(4-fluoro-2- | 345.37 | 346.1062 | 1.22 | C |
| | | | methoxybenzyl)oxy]benzyl}- | | | | |
| | | | 5-methyl-1,3- | | | | |
| | | | oxazolidin-2-one | | | | |
| 148 | <13.4 | nM | 5-methyl-3-(4-{[(1S)- | 325.41 | 326.1291 | 1.32 | C |
| | | | 1- | | | | |
| | | | phenylpropyl]oxy}benzyl)- | | | | |
| | | | 1,3-oxazolidin-2- | | | | |
| | | | one | | | | |
| 149 | 35.8 | nM | 3-{4-[3-(1H-indol-1- | 364.44 | 365.1753 | 1.3 | C |
| | | | yl)propoxy]benzyl}-5- | | | | |
| | | | methyl-1,3- | | | | |
| | | | oxazolidin-2-one | | | | |
| 150 | 101 | nM | 3-{4-[(2-fluoro-5- | 329.37 | 330.0886 | 1.27 | C |
| | | | methylbenzyl)oxy]benzyl}- | | | | |
| | | | 5-methyl-1,3- | | | | |
| | | | oxazolidin-2-one | | | | |
| 151 | 92.1 | nM | 3-{4-[(2-fluoro-5- | 345.37 | 346.1283 | 1.19 | C |
| | | | methoxybenzyl)oxy]benzyl}- | | | | |
| | | | 5-methyl-1,3- | | | | |
| | | | oxazolidin-2-one | | | | |
| 152 | 46.0 | nM | 3-{4-[3-(4- | 359.4 | 360.1061 | 1.28 | C |
| | | | fluorophenoxy)propoxy]benzyl}- | | | | |
| | | | 5-methyl- | | | | |
| | | | 1,3-oxazolidin-2-one | | | | |
| 153 | 28.3 | nM | (5R)-3-[(2′- | 301.77 | 302.14 | 1.37 | D |
| | | | chlorobiphenyl-4- | | | | |
| | | | yl)methyl]-5-methyl- | | | | |
| | | | 1,3-oxazolidin-2-one | | | | |
| 154 | 1240 | nM | (5R)-5-methyl-3-[4- | 353.42 | 354.23 | 0.71 | D |
| | | | (6-morpholin-4- | | | | |
| | | | ylpyridin-3-yl)benzyl]- | | | | |
| | | | 1,3-oxazolidin-2-one | | | | |
| 155 | 489 | nM | (5R)-3-{4-[6- | 311.38 | 312.2 | 0.71 | D |
| | | | (dimethylamino)pyridin- | | | | |
| | | | 3-yl]benzyl}-5- | | | | |
| | | | methyl-1,3- | | | | |
| | | | oxazolidin-2-one | | | | |
| 156 | <16.2 | nM | (5R)-3-[(5′-isopropyl- | 339.43 | 340.21 | 1.53 | D |
| | | | 2′-methoxybiphenyl- | | | | |
| | | | 4-yl)methyl]-5- | | | | |
| | | | methyl-1,3- | | | | |
| | | | oxazolidin-2-one | | | | |
| 157 | <32.5 | nM | (5R)-3-[4-(1- | 307.35 | 308.16 | 1.42 | D |
| | | | benzofuran-2- | | | | |
| | | | yl)benzyl]-5-methyl- | | | | |
| | | | 1,3-oxazolidin-2-one | | | | |
| 158 | 151 | nM | (5R)-5-methyl-3-[4- | 337.42 | 338.26 | 0.76 | D |
| | | | (6-pyrrolidin-1- | | | | |
| | | | ylpyridin-3-yl)benzyl]- | | | | |
| | | | 1,3-oxazolidin-2-one | | | | |
| 159 | 13.9 | nM | (5R)-3-[(4′-fluoro-2′- | 299.34 | 300.19 | 1.39 | D |
| | | | methylbiphenyl-4- | | | | |
| | | | yl)methyl]-5-methyl- | | | | |
| | | | 1,3-oxazolidin-2-one | | | | |
| 160 | 10.6 | nM | (5R)-3-[(4′-fluoro-3′- | 299.34 | 300.19 | 1.42 | D |
| | | | methylbiphenyl-4- | | | | |
| | | | yl)methyl]-5-methyl- | | | | |
| | | | 1,3-oxazolidin-2-one | | | | |
| 161 | 227 | nM | (5R)-3-[(2′,6′- | 327.38 | 328.2 | 1.23 | D |
| | | | dimethoxybiphenyl-4- | | | | |
| | | | yl)methyl]-5-methyl- | | | | |
| | | | 1,3-oxazolidin-2-one | | | | |
| 162 | 42.0 | nM | (5R)-3-(4-isoquinolin- | 318.37 | 319.18 | 0.74 | D |
| | | | 5-ylbenzyl)-5-methyl- | | | | |
| | | | 1,3-oxazolidin-2-one | | | | |
| 163 | <10.0 | nM | (5R)-3-[(3′-chloro-4′- | 319.76 | 320.13 | 1.42 | D |
| | | | fluorobiphenyl-4- | | | | |
| | | | yl)methyl]-5-methyl- | | | | |
| | | | 1,3-oxazolidin-2-one | | | | |
| 164 | <7.32 | nM | (5R)-5-methyl-3-{[2′- | 351.32 | 352.13 | 1.42 | D |
| | | | (trifluoromethoxy)biphenyl- | | | | |
| | | | 4-yl]methyl}- | | | | |
| | | | 1,3-oxazolidin-2-one | | | | |
| 165 | >68.9 | nM | (5R)-3-[(2′,4′- | 303.31 | 304.16 | 1.32 | D |
| | | | difluorobiphenyl-4- | | | | |
| | | | yl)methyl]-5-methyl- | | | | |
| | | | 1,3-oxazolidin-2-one | | | | |
| 166 | 24.6 | nM | (5R)-3-[(5′-fluoro-2′- | 315.34 | 316.15 | 1.29 | D |
| | | | methoxybiphenyl-4- | | | | |
| | | | yl)methyl]-5-methyl- | | | | |
| | | | 1,3-oxazolidin-2-one | | | | |
| 167 | <13.4 | nM | (5R)-3-[(2′- | 295.38 | 296.2 | 1.45 | D |
| | | | ethylbiphenyl-4- | | | | |
| | | | yl)methyl]-5-methyl- | | | | |
| | | | 1,3-oxazolidin-2-one | | | | |
| 168 | 52.6 | nM | (5R)-3-[(4′- | 297.35 | 298.16 | 1.26 | D |
| | | | methoxybiphenyl-4- | | | | |
| | | | yl)methyl]-5-methyl- | | | | |
| | | | 1,3-oxazolidin-2-one | | | | |
| 169 | 49.0 | nM | ethyl 4′-{[(5R)-5- | 339.39 | 340.21 | 1.35 | D |
| | | | methyl-2-oxo-1,3- | | | | |
| | | | oxazolidin-3- | | | | |
| | | | yl]methyl}biphenyl-3- | | | | |
| | | | carboxylate | | | | |
| 170 | 34.2 | nM | (5R)-3-[(3′-fluoro-4′- | 315.34 | 316.16 | 1.26 | D |
| | | | methoxybiphenyl-4- | | | | |
| | | | yl)methyl]-5-methyl- | | | | |
| | | | 1,3-oxazolidin-2-one | | | | |
| 171 | 60.2 | nM | (5R)-3-[(4′- | 309.36 | 310.17 | 1.13 | D |
| | | | acetylbiphenyl-4- | | | | |
| | | | yl)methyl]-5-methyl- | | | | |
| | | | 1,3-oxazolidin-2-one | | | | |
| 172 | 31.4 | nM | (5R)-3-[(2′-fluoro-6′- | 315.34 | 316.15 | 1.26 | D |
| | | | methoxybiphenyl-4- | | | | |
| | | | yl)methyl]-5-methyl- | | | | |
| | | | 1,3-oxazolidin-2-one | | | | |
| 173 | 930 | nM | (5R)-5-methyl-3-(4- | 318.37 | 319.19 | 0.76 | D |
| | | | quinolin-8-ylbenzyl)- | | | | |
| | | | 1,3-oxazolidin-2-one | | | | |
| 174 | 1620 | nM | (5R)-3-[4-(2- | 350.46 | 351.23 | 0.79 | D |
| | | | isopropyl-2,3- | | | | |
| | | | dihydro-1H-isoindol- | | | | |
| | | | 5-yl)benzyl]-5- | | | | |
| | | | methyl-1,3- | | | | |
| | | | oxazolidin-2-one | | | | |
| 175 | 805 | nM | (5R)-3-[(2′- | 309.36 | 310.16 | 1.15 | D |
| | | | acetylbiphenyl-4- | | | | |
| | | | yl)methyl]-5-methyl- | | | | |
| | | | 1,3-oxazolidin-2-one | | | | |
| 176 | 25.8 | nM | (5R)-3-[(2′- | 311.38 | 312.18 | 1.38 | D |
| | | | ethoxybiphenyl-4- | | | | |
| | | | yl)methyl]-5-methyl- | | | | |
| | | | 1,3-oxazolidin-2-one | | | | |
| 177 | 41.3 | nM | (5R)-3-[(2′- | 297.35 | 298.17 | 1.28 | D |
| | | | methoxybiphenyl-4- | | | | |
| | | | yl)methyl]-5-methyl- | | | | |
| | | | 1,3-oxazolidin-2-one | | | | |
| 178 | 523 | nM | (5R)-3-[4-(2,5- | 284.36 | 285.4 | 2.4 | E |
| | | | dimethyl-1H-pyrrol-1- | | | | |
| | | | yl)benzyl]-5-methyl- | | | | |
| | | | 1,3-oxazolidin-2-one | | | | |
| 179 | 137 | nM | (5R)-3-[4-(4- | 301.32 | 302.2 | 3.93 | D |
| | | | fluorophenoxy)benzyl]- | | | | |
| | | | 5-methyl-1,3- | | | | |
| | | | oxazolidin-2-one | | | | |
| 180 | 52.0 | nM | (5R)-5-methyl-3-[(2′- | 281.35 | 282.3 | 4.06 | D |
| | | | methylbiphenyl-4- | | | | |
| | | | yl)methyl]-1,3- | | | | |
| | | | oxazolidin-2-one | | | | |
| 181 | 286 | nM | (5R)-5-methyl-3-(4- | 318.37 | 319.2 | 2.38 | D |
| | | | quinolin-3-ylbenzyl)- | | | | |
| | | | 1,3-oxazolidin-2-one | | | | |
| 182 | 74.6 | nM | (5R)-3-[(3′,4′- | 336.22 | 336.1 | 4.53 | D |
| | | | dichlorobiphenyl-4- | | | | |
| | | | yl)methyl]-5-methyl- | | | | |
| | | | 1,3-oxazolidin-2-one | | | | |
| 183 | 150 | nM | (5R)-3-[4-(4-tert- | 330.45 | 331.3 | 4.44 | D |
| | | | butyl-1,3-thiazol-2- | | | | |
| | | | yl)benzyl]-5-methyl- | | | | |
| | | | 1,3-oxazolidin-2-one | | | | |
| 184 | 964 | nM | (5R)-3-[4- | 325.41 | 326.3 | 4.22 | D |
| | | | (benzyloxy)-3,5- | | | | |
| | | | dimethylbenzyl]-5- | | | | |
| | | | methyl-1,3- | | | | |
| | | | oxazolidin-2-one | | | | |
| 185 | 625 | nM | (5R)-3-(9H-fluoren-2- | 279.34 | 280.3 | 3.94 | D |
| | | | ylmethyl)-5-methyl- | | | | |
| | | | 1,3-oxazolidin-2-one | | | | |
| 186 | 89.0 | nM | (5R)-5-methyl-3-{4- | 311.38 | 312.2 | 4.09 | D |
| | | | [(2- | | | | |
| | | | methylbenzyl)oxy]benzyl}- | | | | |
| | | | 1,3-oxazolidin- | | | | |
| | | | 2-one | | | | |
| 187 | 113 | nM | (5R)-3-[(3′,5′- | 336.22 | 336.2 | 3 | E |
| | | | dichlorobiphenyl-4- | | | | |
| | | | yl)methyl]-5-methyl- | | | | |
| | | | 1,3-oxazolidin-2-one | | | | |
| 188 | 725 | nM | 1-[4-(1- | 344.5 | 345.2 | 2.1 | |
| | | | cyclohexylethoxy)benzyl]- | | | | |
| | | | 4-ethylpiperazin- | | | | |
| | | | 2-one | | | | |
| 189 | 83.7 | nM | (5R)-3-[4-(3,4- | 339.39 | 340.3 | 2.5 | E |
| | | | dihydro-2H-1,5- | | | | |
| | | | benzodioxepin-7- | | | | |
| | | | yl)benzyl]-5-methyl- | | | | |
| | | | 1,3-oxazolidin-2-one | | | | |
| 190 | 676 | nM | (5R)-3-[(3′,4′- | 327.38 | 328.4 | 2.2 | E |
| | | | dimethoxybiphenyl-4- | | | | |
| | | | yl)methyl]-5-methyl- | | | | |
| | | | 1,3-oxazolidin-2-one | | | | |
| 191 | 166 | nM | 4′-{[(5R)-5-methyl-2- | 292.34 | 293.3 | 2.4 | E |
| | | | oxo-1,3-oxazolidin-3- | | | | |
| | | | yl]methyl}biphenyl-4- | | | | |
| | | | carbonitrile | | | | |
| 192 | 280 | nM | (5R)-3-[(3′- | 285.32 | 286.4 | 2.5 | E |
| | | | fluorobiphenyl-4- | | | | |
| | | | yl)methyl]-5-methyl- | | | | |
| | | | 1,3-oxazolidin-2-one | | | | |
| 193 | 514 | nM | (5R)-3-[(3′- | 309.36 | 310.4 | 2.2 | E |
| | | | acetylbiphenyl-4- | | | | |
| | | | yl)methyl]-5-methyl- | | | | |
| | | | 1,3-oxazolidin-2-one | | | | |
| 194 | 275 | nM | (5R)-3-[(3′- | 297.35 | 298.3 | 2.6 | E |
| | | | methoxybiphenyl-4- | | | | |
| | | | yl)methyl]-5-methyl- | | | | |
| | | | 1,3-oxazolidin-2-one | | | | |
| 195 | 158 | nM | (5R)-3-[(3′- | 301.77 | 302.3 | 2.8 | E |
| | | | chlorobiphenyl-4- | | | | |
| | | | yl)methyl]-5-methyl- | | | | |
| | | | 1,3-oxazolidin-2-one | | | | |
| 196 | 48.9 | nM | (5R)-3-[(5′-chloro-2′- | 331.8 | 332.3 | 2.7 | E |
| | | | methoxybiphenyl-4- | | | | |
| | | | yl)methyl]-5-methyl- | | | | |
| | | | 1,3-oxazolidin-2-one | | | | |
| 197 | 1830 | nM | ethyl 4′-{[(5R)-5- | 339.39 | 340.4 | 2.4 | E |
| | | | methyl-2-oxo-1,3- | | | | |
| | | | oxazolidin-3- | | | | |
| | | | yl]methyl}biphenyl-2- | | | | |
| | | | carboxylate | | | | |
| 198 | 42.2 | nM | (5R)-3-[4-(2,3- | 325.36 | 326.4 | 2.4 | E |
| | | | dihydro-1,4- | | | | |
| | | | benzodioxin-6- | | | | |
| | | | yl)benzyl]-5-methyl- | | | | |
| | | | 1,3-oxazolidin-2-one | | | | |
| 199 | 804 | nM | (5R)-3-{[3′-(3,5- | 361.44 | 362.4 | 2.5 | E |
| | | | dimethyl-1H-pyrazol- | | | | |
| | | | 1-yl)biphenyl-4- | | | | |
| | | | yl]methyl}-5-methyl- | | | | |
| | | | 1,3-oxazolidin-2-one | | | | |
| 200 | 191 | nM | 1-[4-(1- | 372.55 | 373.3 | 2.6 | |
| | | | cyclohexylethoxy)benzyl]- | | | | |
| | | | 4- | | | | |
| | | | isobutylpiperazin-2- | | | | |
| | | | one | | | | |
| 201 | 763 | nM | 1-[4-(1- | 358.52 | 359.3 | 2.2 | |
| | | | cyclohexylethoxy)benzyl]- | | | | |
| | | | 4- | | | | |
| | | | isopropylpiperazin-2- | | | | |
| | | | one | | | | |
| 202 | 291 | nM | 1-[4-(1- | 398.59 | 399.3 | 2.6 | |
| | | | cyclohexylethoxy)benzyl]- | | | | |
| | | | 4- | | | | |
| | | | (cyclopentylmethyl)piperazin- | | | | |
| | | | 2-one | | | | |
| 203 | <11.5 | nM | (5R)-3-[4- | 303.4 | 304.4 | 2.9 | E |
| | | | (cyclohexylmethoxy)benzyl]- | | | | |
| | | | 5-methyl-1,3- | | | | |
| | | | oxazolidin-2-one | | | | |
| 204 | 24.8 | nM | (5R)-5-methyl-3-[4- | 325.41 | 326.4 | 2.8 | E |
| | | | (3- | | | | |
| | | | phenylpropoxy)benzyl]- | | | | |
| | | | 1,3-oxazolidin-2- | | | | |
| | | | one | | | | |
| 205 | 68.0 | nM | (5R)-3-{4-[(5-fluoro- | 329.37 | 330.4 | 2.7 | E |
| | | | 2- | | | | |
| | | | methylbenzyl)oxy]benzyl}- | | | | |
| | | | 5-methyl-1,3- | | | | |
| | | | oxazolidin-2-one | | | | |
| 206 | 29.9 | nM | (5R)-3-{4-[3-(4- | 359.4 | 360.4 | 2.7 | E |
| | | | fluorophenoxy)propoxy]benzyl}- | | | | |
| | | | 5-methyl- | | | | |
| | | | 1,3-oxazolidin-2-one | | | | |
| 207 | 63.5 | nM | 3-[4-(1- | 380.49 | 381.4 | 3.1 | E |
| | | | cyclohexylethoxy)benzyl]- | | | | |
| | | | 5-pyridin-2-yl- | | | | |
| | | | 1,3-oxazolidin-2-one | | | | |
| 208 | 41.6 | nM | 3-[4-(1- | 380.49 | 381.4 | 2.8 | E |
| | | | cyclohexylethoxy)benzyl]- | | | | |
| | | | 5-pyridin-3-yl- | | | | |
| | | | 1,3-oxazolidin-2-one | | | | |
| 209 | 639 | nM | 3-[4-(1- | 380.49 | 381.4 | 2.8 | E |
| | | | cyclohexylethoxy)benzyl]- | | | | |
| | | | 5-pyridin-4-yl- | | | | |
| | | | 1,3-oxazolidin-2-one | | | | |
| 210 | 424 | nM | (5R)-3-{[6-(4-chloro- | 320.75 | 321.3 | 2.4 | E |
| | | | 2- | | | | |
| | | | fluorophenyl)pyridin- | | | | |
| | | | 3-yl]methyl}-5- | | | | |
| | | | methyl-1,3- | | | | |
| | | | oxazolidin-2-one | | | | |
| 211 | 88.0 | nM | (5R)-3-{[6-(1- | 318.41 | 319.5 | 2.9 | E |
| | | | cyclohexylethoxy)pyridin- | | | | |
| | | | 3-yl]methyl}-5- | | | | |
| | | | methyl-1,3- | | | | |
| | | | oxazolidin-2-one | | | | |
| 212 | 77.8 | nM | (5R)-5-methyl-3-{4- | 291.39 | 292.5 | 2.9 | E |
| | | | [(3- | | | | |
| | | | methylpentyl)oxy]benzyl}- | | | | |
| | | | 1,3-oxazolidin-2- | | | | |
| | | | one | | | | |
| 213 | 20.1 | nM | (5R)-3-[4-(3,3- | 291.39 | 292.5 | 2.9 | E |
| | | | dimethylbutoxy)benzyl]- | | | | |
| | | | 5-methyl-1,3- | | | | |
| | | | oxazolidin-2-one | | | | |
| 214 | 712 | nM | 1-[4-(1- | 400.6 | 401.3 | 3.44 | F |
| | | | cyclohexylethoxy)benzyl]- | | | | |
| | | | 4-(2- | | | | |
| | | | methylpentyl)piperazin- | | | | |
| | | | 2-one | | | | |
| 215 | 435 | nM | 1-[4-(1- | 358.52 | 359.3 | 3.1 | F |
| | | | cyclohexylethoxy)benzyl]- | | | | |
| | | | 4- | | | | |
| | | | propylpiperazin-2- | | | | |
| | | | one | | | | |
| 216 | 471 | nM | 1-[4-(1- | 420.59 | 421.3 | 3.38 | F |
| | | | cyclohexylethoxy)benzyl]- | | | | |
| | | | 4-(4- | | | | |
| | | | methylbenzyl)piperazin- | | | | |
| | | | 2-one | | | | |
| 217 | 947 | nM | 1-[4-(1- | 400.6 | 401.3 | 3.42 | F |
| | | | cyclohexylethoxy)benzyl]- | | | | |
| | | | 4-(2- | | | | |
| | | | ethylbutyl)piperazin- | | | | |
| | | | 2-one | | | | |
| 218 | 681 | nM | 1-[4-(1- | 426.52 | 427.3 | 3.27 | F |
| | | | cyclohexylethoxy)benzyl]- | | | | |
| | | | 4-(4,4,4- | | | | |
| | | | trifluorobutyl)piperazin- | | | | |
| | | | 2-one | | | | |
| 219 | 697 | nM | 1-[4-(1- | 398.59 | 398.8; | 1.33; 3.41 | F |
| | | | cyclohexylethoxy)benzyl]- | | 399.3 | | |
| | | | 4-(2-cyclopropyl- | | | | |
| | | | 1- | | | | |
| | | | methylethyl)piperazin- | | | | |
| | | | 2-one | | | | |
| 220 | 801 | nM | 1-[4-(1- | 386.58 | 387.3 | 3.28 | F |
| | | | cyclohexylethoxy)benzyl]- | | | | |
| | | | 4-(3- | | | | |
| | | | methylbutyl)piperazin- | | | | |
| | | | 2-one | | | | |
| 221 | 794 | nM | 4-(1-cyclobutylethyl)- | 398.59 | 399.3; | 3.27; 3.48 | F |
| | | | 1-[4-(1- | | 399.4 | | |
| | | | cyclohexylethoxy)benzyl]piperazin- | | | | |
| | | | 2-one | | | | |
| 222 | 325 | nM | 1-[4-(1- | 412.49 | 413.3 | 3.52 | F |
| | | | cyclohexylethoxy)benzyl]- | | | | |
| | | | 4-(3,3,3- | | | | |
| | | | trifluoropropyl)piperazin- | | | | |
| | | | 2-one | | | | |
| 223 | 584 | nM | 1-[4-(1- | 386.58 | 387.3; | 3.3; 3.51 | F |
| | | | cyclohexylethoxy)benzyl]- | | 387.3 | | |
| | | | 4-(2,2- | | | | |
| | | | dimethylpropyl)piperazin- | | | | |
| | | | 2-one | | | | |
| 224 | 1100 | nM | 1-[4-(1- | 398.59 | 399.3; | 3.27; 3.48 | F |
| | | | cyclohexylethoxy)benzyl]- | | 399.4 | | |
| | | | 4-[(3R)-3- | | | | |
| | | | methylcyclopentyl]piperazin- | | | | |
| | | | 2-one | | | | |
| 225 | 638 | nM | 1-[4-(1- | 398.59 | 399.3; | 3.32; 3.5 | F |
| | | | cyclohexylethoxy)benzyl]- | | 399.2 | | |
| | | | 4-(2- | | | | |
| | | | methylcyclopentyl)piperazin- | | | | |
| | | | 2-one | | | | |
| 226 | 32.3 | nM | (5R)-3-{[2′-fluoro-4′- | 369.31 | 370.3 | 2.9 | E |
| | | | (trifluoromethoxy)biphenyl- | | | | |
| | | | 4-yl]methyl}-5- | | | | |
| | | | methyl-1,3- | | | | |
| | | | oxazolidin-2-one | | | | |
| 227 | 196 | nM | (5R)-3-[4- | 321.39 | 322.4 | 3 | E |
| | | | (cyclohexylmethoxy)- | | | | |
| | | | 3-fluorobenzyl]-5- | | | | |
| | | | methyl-1,3- | | | | |
| | | | oxazolidin-2-one | | | | |
| 228 | 870 | nM | (5R)-3-(3-fluoro-4- | 401.33 | 402.3 | 2.9 | E |
| | | | {[2-fluoro-4- | | | | |
| | | | (trifluoromethyl)benzyl]oxy}benzyl)- | | | | |
| | | | 5- | | | | |
| | | | methyl-1,3- | | | | |
| | | | oxazolidin-2-one | | | | |
| 229 | 757 | nM | (5R)-3-({6-[3-(4- | 360.38 | 361.4 | 2.6 | E |
| | | | fluorophenoxy)propoxy]pyridin- | | | | |
| | | | 3- | | | | |
| | | | yl}methyl)-5-methyl- | | | | |
| | | | 1,3-oxazolidin-2-one | | | | |
| 230 | <21.6 | nM | (5R)-3-[4-(1- | 335.42 | 336.4 | 3 | E |
| | | | cyclohexylethoxy)-3- | | | | |
| | | | fluorobenzyl]-5- | | | | |
| | | | methyl-1,3- | | | | |
| | | | oxazolidin-2-one | | | | |
| 231 | 683 | nM | 6-chloro-4′-{[(5R)-5- | 326.78 | 327.4 | 2.4 | E |
| | | | methyl-2-oxo-1,3- | | | | |
| | | | oxazolidin-3- | | | | |
| | | | yl]methyl}biphenyl-3- | | | | |
| | | | carbonitrile | | | | |
| 232 | <39.2 | nM | (5R)-3-[(2′-fluoro-4′- | 299.34 | 300.4 | 2.8 | E |
| | | | methylbiphenyl-4- | | | | |
| | | | yl)methyl]-5-methyl- | | | | |
| | | | 1,3-oxazolidin-2-one | | | | |
| 233 | 45.6 | nM | (5R)-3-[(5′-chloro-2′- | 315.8 | 316.4 | 2.9 | E |
| | | | methylbiphenyl-4- | | | | |
| | | | yl)methyl]-5-methyl- | | | | |
| | | | 1,3-oxazolidin-2-one | | | | |
| 234 | <34.5 | nM | (5R)-3-{[2′-fluoro-4′- | 353.31 | 354.4 | 2.8 | E |
| | | | (trifluoromethyl)biphenyl- | | | | |
| | | | 4-yl]methyl}-5- | | | | |
| | | | methyl-1,3- | | | | |
| | | | oxazolidin-2-one | | | | |
| 235 | <23.5 | nM | (5R)-3-[(3′-chloro-2′- | 319.76 | 320.4 | 2.7 | E |
| | | | fluorobiphenyl-4- | | | | |
| | | | yl)methyl]-5-methyl- | | | | |
| | | | 1,3-oxazolidin-2-one | | | | |
| 236 | 49.0 | nM | (5R)-3-[(4′-chloro-2′- | 315.8 | 316.4 | 3 | E |
| | | | methylbiphenyl-4- | | | | |
| | | | yl)methyl]-5-methyl- | | | | |
| | | | 1,3-oxazolidin-2-one | | | | |
| 237 | 112 | nM | (5R)-3-[(4′-chloro-3′- | 319.76 | 320.3 | 2.8 | E |
| | | | fluorobiphenyl-4- | | | | |
| | | | yl)methyl]-5-methyl- | | | | |
| | | | 1,3-oxazolidin-2-one | | | | |
| 238 | 183 | nM | (5R)-5-methyl-3-{[4′- | 351.32 | 352.4 | 2.9 | E |
| | | | (trifluoromethoxy)biphenyl- | | | | |
| | | | 4-yl]methyl}- | | | | |
| | | | 1,3-oxazolidin-2-one | | | | |
| 239 | <105 | nM | (5R)-3-[(4′-fluoro-2′- | 315.34 | 316.4 | 2.5 | E |
| | | | methoxybiphenyl-4- | | | | |
| | | | yl)methyl]-5-methyl- | | | | |
| | | | 1,3-oxazolidin-2-one | | | | |
| 240 | 120 | nM | (5R)-3-[(2′-fluoro-5′- | 315.34 | 316.4 | 2.5 | E |
| | | | methoxybiphenyl-4- | | | | |
| | | | yl)methyl]-5-methyl- | | | | |
| | | | 1,3-oxazolidin-2-one | | | | |
| 241 | 418 | nM | (5R)-3-[4-(2- | 335.42 | 336.5 | 3.1 | E |
| | | | cyclohexylethoxy)-3- | | | | |
| | | | fluorobenzyl]-5- | | | | |
| | | | methyl-1,3- | | | | |
| | | | oxazolidin-2-one | | | | |
| 242 | <17.2 | nM | (5R)-3-[(2′,3′-difluoro- | 333.33 | 334.4 | 2.5 | E |
| | | | 4′-methoxybiphenyl- | | | | |
| | | | 4-yl)methyl]-5- | | | | |
| | | | methyl-1,3- | | | | |
| | | | oxazolidin-2-one | | | | |
| 243 | <25.3 | nM | (5R)-3-[(2′,3′-difluoro- | 333.33 | 334.4 | 2.5 | E |
| | | | 6′-methoxybiphenyl- | | | | |
| | | | 4-yl)methyl]-5- | | | | |
| | | | methyl-1,3- | | | | |
| | | | oxazolidin-2-one | | | | |
| 244 | 97.2 | nM | (5R)-3-[(2′,3′- | 303.31 | 304.3 | 2.5 | E |
| | | | difluorobiphenyl-4- | | | | |
| | | | yl)methyl]-5-methyl- | | | | |
| | | | 1,3-oxazolidin-2-one | | | | |
| 245 | 92.9 | nM | (5R)-3-[(3′,4′- | 303.31 | 304.4 | 2.6 | E |
| | | | difluorobiphenyl-4- | | | | |
| | | | yl)methyl]-5-methyl- | | | | |
| | | | 1,3-oxazolidin-2-one | | | | |
| 246 | 445 | nM | (5R)-3-[(3′,5′- | 303.31 | 304.3 | 2.8 | E |
| | | | difluorobiphenyl-4- | | | | |
| | | | yl)methyl]-5-methyl- | | | | |
| | | | 1,3-oxazolidin-2-one | | | | |
| 247 | 211 | nM | (5R)-3-[(2′,6′- | 303.31 | 304.3 | 2.6 | E |
| | | | difluorobiphenyl-4- | | | | |
| | | | yl)methyl]-5-methyl- | | | | |
| | | | 1,3-oxazolidin-2-one | | | | |
| 248 | 53.7 | nM | (5R)-3-[(5′-fluoro-2′- | 299.34 | 300.4 | 2.6 | E |
| | | | methylbiphenyl-4- | | | | |
| | | | yl)methyl]-5-methyl- | | | | |
| | | | 1,3-oxazolidin-2-one | | | | |
| 249 | <28.4 | nM | (5R)-5-methyl-3- | 321.3 | 322.3 | 2.6 | E |
| | | | [(2′,3,4′- | | | | |
| | | | trifluorobiphenyl-4- | | | | |
| | | | yl)methyl]-1,3- | | | | |
| | | | oxazolidin-2-one | | | | |
| 250 | 1870 | nM | 3-[4- | 380.49 | 381.4 | 2.9 | E |
| | | | (cyclohexylmethoxy)- | | | | |
| | | | 3-methylbenzyl]-5- | | | | |
| | | | pyridin-3-yl-1,3- | | | | |
| | | | oxazolidin-2-one | | | | |
| 251 | 55.9 | nM | (5R)-3-[(2′-chloro- | 337.75 | 338.3 | 2.7 | E |
| | | | 3,3′-difluorobiphenyl- | | | | |
| | | | 4-yl)methyl]-5- | | | | |
| | | | methyl-1,3- | | | | |
| | | | oxazolidin-2-one | | | | |
| 252 | 29.3 | nM | (5R)-3-{[2′,3-difluoro- | 371.3 | 372.3 | 2.8 | E |
| | | | 4′- | | | | |
| | | | (trifluoromethyl)biphenyl- | | | | |
| | | | 4-yl]methyl}-5- | | | | |
| | | | methyl-1,3- | | | | |
| | | | oxazolidin-2-one | | | | |
| 253 | 135 | nM | (5R)-3-[(3,4′-difluoro- | 333.33 | 334.4 | 2.6 | E |
| | | | 2-methoxybiphenyl- | | | | |
| | | | 4-yl)methyl]-5- | | | | |
| | | | methyl-1,3- | | | | |
| | | | oxazolidin-2-one | | | | |
| 254 | 71.2 | nM | (5R)-3-[4- | 317.43 | 318.4 | 3.1 | E |
| | | | (cyclohexylmethoxy)- | | | | |
| | | | 3-methylbenzyl]-5- | | | | |
| | | | methyl-1,3- | | | | |
| | | | oxazolidin-2-one | | | | |
| 255 | 463 | nM | (5R)-5-methyl-3-({6- | 352.31 | NA | NA | NA |
| | | | [4- | | | | |
| | | | (trifluoromethoxy)phenyl]pyridin- | | | | |
| | | | 3- | | | | |
| | | | yl}methyl)-1,3- | | | | |
| | | | oxazolidin-2-one | | | | |
| 256 | <39.8 | nM | (5R)-3-[(4′-chloro-2′- | 319.76 | 320.3 | 2.8 | E |
| | | | fluorobiphenyl-4- | | | | |
| | | | yl)methyl]-5-methyl- | | | | |
| | | | 1,3-oxazolidin-2-one | | | | |
| 257 | 429 | nM | (5R)-3-{[6-(2-fluoro- | 300.33 | 301.4 | 2.1 | E |
| | | | 4- | | | | |
| | | | methylphenyl)pyridin- | | | | |
| | | | 3-yl]methyl}-5- | | | | |
| | | | methyl-1,3- | | | | |
| | | | oxazolidin-2-one | | | | |
| 258 | 205 | nM | (5R)-3-({6-[2-fluoro- | 354.3 | 355.4 | 2.4 | E |
| | | | 4- | | | | |
| | | | (trifluoromethyl)phenyl]pyridin- | | | | |
| | | | 3- | | | | |
| | | | yl}methyl)-5-methyl- | | | | |
| | | | 1,3-oxazolidin-2-one | | | | |
| 259 | 1460 | nM | (5R)-3-{[6-(5-chloro- | 316.79 | 317.4 | 2.3 | E |
| | | | 2- | | | | |
| | | | methylphenyl)pyridin- | | | | |
| | | | 3-yl]methyl}-5- | | | | |
| | | | methyl-1,3- | | | | |
| | | | oxazolidin-2-one | | | | |
| 260 | 858 | nM | (5R)-3-{[6-(2-fluoro- | 316.33 | 317.4 | 2.1 | E |
| | | | 5- | | | | |
| | | | methoxyphenyl)pyridin- | | | | |
| | | | 3-yl]methyl}-5- | | | | |
| | | | methyl-1,3- | | | | |
| | | | oxazolidin-2-one | | | | |
| 261 | 116 | nM | (5R)-3-{[6-(3,4- | 337.21 | 337.3 | 2.6 | E |
| | | | dichlorophenyl)pyridin- | | | | |
| | | | 3-yl]methyl}-5- | | | | |
| | | | methyl-1,3- | | | | |
| | | | oxazolidin-2-one | | | | |
| 262 | 363 | nM | (5R)-3-{[6-(2,3- | 334.32 | 335.4 | 2.1 | E |
| | | | difluoro-4- | | | | |
| | | | methoxyphenyl)pyridin- | | | | |
| | | | 3-yl]methyl}-5- | | | | |
| | | | methyl-1,3- | | | | |
| | | | oxazolidin-2-one | | | | |
| 263 | 1370 | nM | (5R)-3-{[6-(2,3- | 334.32 | 335.4 | 1.9 | E |
| | | | difluoro-6- | | | | |
| | | | methoxyphenyl)pyridin- | | | | |
| | | | 3-yl]methyl}-5- | | | | |
| | | | methyl-1,3- | | | | |
| | | | oxazolidin-2-one | | | | |
| 264 | 561 | nM | (5R)-3-{[6-(2,3- | 310.35 | 311.4 | 1.6 | E |
| | | | dihydro-1- | | | | |
| | | | benzofuran-5- | | | | |
| | | | yl)pyridin-3- | | | | |
| | | | yl]methyl}-5-methyl- | | | | |
| | | | 1,3-oxazolidin-2-one | | | | |
| 265 | 1100 | nM | (5R)-5-methyl-3-({6- | 336.31 | 337.4 | 2.5 | E |
| | | | [4- | | | | |
| | | | (trifluoromethyl)phenyl]pyridin- | | | | |
| | | | 3- | | | | |
| | | | yl}methyl)-1,3- | | | | |
| | | | oxazolidin-2-one | | | | |
| 266 | 562 | nM | (5R)-3-{[6-(5-chloro- | 332.79 | 333.4 | 2.1 | E |
| | | | 2- | | | | |
| | | | methoxyphenyl)pyridin- | | | | |
| | | | 3-yl]methyl}-5- | | | | |
| | | | methyl-1,3- | | | | |
| | | | oxazolidin-2-one | | | | |
| 267 | 1160 | nM | (5R)-3-{[6-(5-fluoro- | 316.33 | 317.4 | 1.9 | E |
| | | | 2- | | | | |
| | | | methoxyphenyl)pyridin- | | | | |
| | | | 3-yl]methyl}-5- | | | | |
| | | | methyl-1,3- | | | | |
| | | | oxazolidin-2-one | | | | |
| 268 | 102 | nM | (5R)-3-({6-[2-chloro- | 370.76 | 371.3 | 2.5 | E |
| | | | 4- | | | | |
| | | | (trifluoromethyl)phenyl]pyridin- | | | | |
| | | | 3- | | | | |
| | | | yl}methyl)-5-methyl- | | | | |
| | | | 1,3-oxazolidin-2-one | | | | |
| 269 | 518 | nM | (5R)-3-{[6-(2,4- | 337.21 | 337.3 | 2.4 | E |
| | | | dichlorophenyl)pyridin- | | | | |
| | | | 3-yl]methyl}-5- | | | | |
| | | | methyl-1,3- | | | | |
| | | | oxazolidin-2-one | | | | |
| 270 | 595 | nM | (5R)-3-{[6-(3-chloro- | 320.75 | 321.3 | 2.5 | E |
| | | | 4- | | | | |
| | | | fluorophenyl)pyridin- | | | | |
| | | | 3-yl]methyl}-5- | | | | |
| | | | methyl-1,3- | | | | |
| | | | oxazolidin-2-one | | | | |
| 271 | 475 | nM | (5R)-3-{[6-(4-fluoro- | 300.33 | 301.4 | 2.3 | E |
| | | | 3- | | | | |
| | | | methylphenyl)pyridin- | | | | |
| | | | 3-yl]methyl}-5- | | | | |
| | | | methyl-1,3- | | | | |
| | | | oxazolidin-2-one | | | | |
| 272 | 20.1 | nM | (5R)-3-{[2′-chloro-4′- | 369.77 | 370.3 | 3 | E |
| | | | (trifluoromethyl)biphenyl- | | | | |
| | | | 4-yl]methyl}-5- | | | | |
| | | | methyl-1,3- | | | | |
| | | | oxazolidin-2-one | | | | |
| 273 | <40.1 | nM | (5R)-3-[(2′,4′- | 336.22 | 336.3 | 2.9 | E |
| | | | dichlorobiphenyl-4- | | | | |
| | | | yl)methyl]-5-methyl- | | | | |
| | | | 1,3-oxazolidin-2-one | | | | |
| 274 | 31.3 | nM | (5R)-3-[(2′-chloro-4′- | 319.76 | 320.3 | 2.7 | E |
| | | | fluorobiphenyl-4- | | | | |
| | | | yl)methyl]-5-methyl- | | | | |
| | | | 1,3-oxazolidin-2-one | | | | |
| 275 | 47.4 | nM | (5R)-3-{[2′,4′- | 403.32 | 404.3 | 3.1 | E |
| | | | bis(trifluoromethyl)biphenyl- | | | | |
| | | | 4-yl]methyl}-5- | | | | |
| | | | methyl-1,3- | | | | |
| | | | oxazolidin-2-one | | | | |
| 276 | 788 | nM | (5R)-3-({6-[2,4- | 404.31 | 405.3 | 2.7 | E |
| | | | bis(trifluoromethyl)phenyl]pyridin- | | | | |
| | | | 3- | | | | |
| | | | yl}methyl)-5-methyl- | | | | |
| | | | 1,3-oxazolidin-2-one | | | | |
| 277 | 53.1 | nM | (5R)-3-[(3′-fluoro-4′- | 343.4 | 344.5 | 2.8 | E |
| | | | isopropoxybiphenyl- | | | | |
| | | | 4-yl)methyl]-5- | | | | |
| | | | methyl-1,3- | | | | |
| | | | oxazolidin-2-one | | | | |
| 278 | 164 | nM | (5R)-3-{[6-(2-chloro- | 360.84 | 361.4 | 2.4 | E |
| | | | 4- | | | | |
| | | | isopropoxyphenyl)pyridin- | | | | |
| | | | 3-yl]methyl}-5- | | | | |
| | | | methyl-1,3- | | | | |
| | | | oxazolidin-2-one | | | | |
| 279 | 159 | nM | (5R)-3-{[6-(2-fluoro- | 344.38 | 345.4 | 2.4 | E |
| | | | 4- | | | | |
| | | | isopropoxyphenyl)pyridin- | | | | |
| | | | 3-yl]methyl}-5- | | | | |
| | | | methyl-1,3- | | | | |
| | | | oxazolidin-2-one | | | | |
| 280 | <15.4 | nM | (5R)-3-[(2′-chloro-4′- | 359.85 | 360.4 | 3 | E |
| | | | isopropoxybiphenyl- | | | | |
| | | | 4-yl)methyl]-5- | | | | |
| | | | methyl-1,3- | | | | |
| | | | oxazolidin-2-one | | | | |
| 281 | 8.14 | nM | (5R)-3-[(2′-fluoro-4′- | 343.4 | 344.4 | 2.9 | E |
| | | | isopropoxybiphenyl- | | | | |
| | | | 4-yl)methyl]-5- | | | | |
| | | | methyl-1,3- | | | | |
| | | | oxazolidin-2-one | | | | |
| 282 | 1470 | nM | (5R)-3-{[6-(5- | 340.42 | 341.5 | 2.1 | E |
| | | | isopropyl-2- | | | | |
| | | | methoxyphenyl)pyridin- | | | | |
| | | | 3-yl]methyl}-5- | | | | |
| | | | methyl-1,3- | | | | |
| | | | oxazolidin-2-one | | | | |
| 283 | 1290 | nM | (5R)-3-({6- | 303.4 | 304.5 | 1.5 | E |
| | | | [(cyclohexylmethyl)amino]pyridin- | | | | |
| | | | 3- | | | | |
| | | | yl}methyl)-5-methyl- | | | | |
| | | | 1,3-oxazolidin-2-one | | | | |
| 284 | 309 | nM | (5R)-5-methyl-3-(3- | 379.38 | 380.3 | 3 | E |
| | | | methyl-4-{[4- | | | | |
| | | | (trifluoromethyl)benzyl]oxy}benzyl)- | | | | |
| | | | 1,3- | | | | |
| | | | oxazolidin-2-one | | | | |
| 285 | 217 | nM | (5R)-3-(4-{[4-fluoro- | 397.37 | 398.3 | 3.1 | E |
| | | | 2- | | | | |
| | | | (trifluoromethyl)benzyl]oxy}- | | | | |
| | | | 3- | | | | |
| | | | methylbenzyl)-5- | | | | |
| | | | methyl-1,3- | | | | |
| | | | oxazolidin-2-one | | | | |
| 286 | 32.5 | nM | (5R)-3-[3-chloro-4- | 337.85 | 338.2 | 3.2 | E |
| | | | (cyclohexylmethoxy)benzyl]- | | | | |
| | | | 5-methyl-1,3- | | | | |
| | | | oxazolidin-2-one | | | | |
| 287 | 174 | nM | (5R)-3-[3-chloro-4-(2- | 351.87 | 352.4 | 3.3 | E |
| | | | cyclohexylethoxy)benzyl]- | | | | |
| | | | 5-methyl-1,3- | | | | |
| | | | oxazolidin-2-one | | | | |
| 288 | 36.9 | nM | (5R)-5-methyl-3- | 321.3 | 322.3 | 2.5 | E |
| | | | [(2,2′,4′- | | | | |
| | | | trifluorobiphenyl-4- | | | | |
| | | | yl)methyl]-1,3- | | | | |
| | | | oxazolidin-2-one | | | | |
| 289 | 93.7 | nM | (5R)-5-methyl-3- | 321.3 | 322.3 | 2.6 | E |
| | | | [(2,2′,3′- | | | | |
| | | | trifluorobiphenyl-4- | | | | |
| | | | yl)methyl]-1,3- | | | | |
| | | | oxazolidin-2-one | | | | |
| 290 | 130 | nM | (5R)-3-[(2,4′-difluoro- | 317.33 | 318.4 | 2.7 | E |
| | | | 2′-methylbiphenyl-4- | | | | |
| | | | yl)methyl]-5-methyl- | | | | |
| | | | 1,3-oxazolidin-2-one | | | | |
| 291 | 59.6 | nM | (5R)-3-[(3′-chloro- | 337.75 | 338.3 | 2.7 | E |
| | | | 2,2′-difluorobiphenyl- | | | | |
| | | | 4-yl)methyl]-5- | | | | |
| | | | methyl-1,3- | | | | |
| | | | oxazolidin-2-one | | | | |
| 292 | 56.0 | nM | (5R)-3-{[2,2′-difluoro- | 371.3 | 372.3 | 3 | E |
| | | | 4′- | | | | |
| | | | (trifluoromethyl)biphenyl- | | | | |
| | | | 4-yl]methyl}-5- | | | | |
| | | | methyl-1,3- | | | | |
| | | | oxazolidin-2-one | | | | |
| 293 | <59.9 | nM | (5R)-5-methyl-3- | 351.32 | 352.4 | 2.6 | E |
| | | | [(2,2′,3′-trifluoro-4′- | | | | |
| | | | methoxybiphenyl-4- | | | | |
| | | | yl)methyl]-1,3- | | | | |
| | | | oxazolidin-2-one | | | | |
| 294 | 327 | nM | (5R)-3-(3-fluoro-4- | 336.36 | 337.3 | 2.2 | E |
| | | | quinolin-3-ylbenzyl)- | | | | |
| | | | 5-methyl-1,3- | | | | |
| | | | oxazolidin-2-one | | | | |
| 295 | 804 | nM | (5R)-3-[3-fluoro-4-(6- | 316.33 | 317.4 | 2.3 | E |
| | | | methoxypyridin-3- | | | | |
| | | | yl)benzyl]-5-methyl- | | | | |
| | | | 1,3-oxazolidin-2-one | | | | |
| 296 | 45.5 | nM | (5R)-3-{4-[(3,5- | 367.23 | 367.3 | 2.9 | E |
| | | | dichloropyridin-2- | | | | |
| | | | yl)oxy]-3- | | | | |
| | | | methylbenzyl}-5- | | | | |
| | | | methyl-1,3- | | | | |
| | | | oxazolidin-2-one | | | | |
| 297 | 1990 | nM | (5R)-3-(4-{[4- | 372.51 | 373.5 | 1.6 | E |
| | | | (cyclobutylamino)cyclohexyl]methoxy}benzyl)- | | | | |
| | | | 5-methyl-1,3- | | | | |
| | | | oxazolidin-2-one | | | | |
| 298 | 1360 | nM | (5R)-3-{[6-(2,3- | 322.29 | 323.3 | 2.3 | E |
| | | | difluorophenyl)-5- | | | | |
| | | | fluoropyridin-3- | | | | |
| | | | yl]methyl}-5-methyl- | | | | |
| | | | 1,3-oxazolidin-2-one | | | | |
| 299 | 167 | nM | (5R)-3-[(2′,4′-difluoro- | 317.33 | 318.3 | 2.7 | E |
| | | | 2-methylbiphenyl-4- | | | | |
| | | | yl)methyl]-5-methyl- | | | | |
| | | | 1,3-oxazolidin-2-one | | | | |
| 300 | 94.5 | nM | (5R)-3-{4-[(5-chloro- | 356.85 | 357.3 | 2.9 | E |
| | | | 2,3-dihydro-1H-indol- | | | | |
| | | | 1-yl)methyl]benzyl}- | | | | |
| | | | 5-methyl-1,3- | | | | |
| | | | oxazolidin-2-one | | | | |
| 301 | 389 | nM | (5R)-3-[4-(2,3- | 322.41 | 323.4 | 2.8 | E |
| | | | dihydro-1H-indol-1- | | | | |
| | | | ylmethyl)benzyl]-5- | | | | |
| | | | methyl-1,3- | | | | |
| | | | oxazolidin-2-one | | | | |
| 302 | 78.9 | nM | 3-[(2′,4′- | 366.37 | 367.3 | 2.5 | E |
| | | | difluorobiphenyl-4- | | | | |
| | | | yl)methyl]-5-pyridin- | | | | |
| | | | 3-yl-1,3-oxazolidin-2- | | | | |
| | | | one | | | | |
| 303 | 77.7 | nM | 3-{[2′-fluoro-4′- | 432.37 | 433.3 | 2.7 | E |
| | | | (trifluoromethoxy)biphenyl- | | | | |
| | | | 4-yl]methyl}-5- | | | | |
| | | | pyridin-3-yl-1,3- | | | | |
| | | | oxazolidin-2-one | | | | |
| 304 | 581 | nM | 3-[(2′,4′- | 360.4 | 361.4 | 1.9 | E |
| | | | difluorobiphenyl-4- | | | | |
| | | | yl)methyl]-5- | | | | |
| | | | {[ethyl(methyl)amino]methyl}- | | | | |
| | | | 1,3- | | | | |
| | | | oxazolidin-2-one | | | | |
| 305 | 416 | nM | (5R)-3-{4-[(2,4- | 333.33 | 334.3 | 2.7 | E |
| | | | difluorophenoxy)methyl]benzyl}- | | | | |
| | | | 5-methyl- | | | | |
| | | | 1,3-oxazolidin-2-one | | | | |
| 306 | 327 | nM | 4-(1-cyclobutylethyl)- | 384.47 | 385 | 2.64 | E |
| | | | 1-[(2′,4′- | | | | |
| | | | difluorobiphenyl-4- | | | | |
| | | | yl)methyl]piperazin-2- | | | | |
| | | | one | | | | |
| 307 | 1360 | nM | 3-[(2′,4′- | 346.38 | 347.3 | 1.99 | E |
| | | | difluorobiphenyl-4- | | | | |
| | | | yl)methyl]-5- | | | | |
| | | | [(dimethylamino)methyl]- | | | | |
| | | | 1,3-oxazolidin-2- | | | | |
| 312 | 578 | nM | (5R)-3-({6-[2-chloro- | 368.76 | 369.3 | 2.25 | E |
| | | | 4- | | | | |
| | | | (difluoromethoxy)phenyl]pyridin- | | | | |
| | | | 3- | | | | |
| | | | yl}methyl)-5-methyl- | | | | |
| | | | 1,3-oxazolidin-2-one | | | | |
| 313 | 531 | nM | (5R)-3-({6-[4- | 334.32 | 335.3 | 2.07 | E |
| | | | (difluoromethoxy)phenyl]pyridin- | | | | |
| | | | 3- | | | | |
| | | | yl}methyl)-5-methyl- | | | | |
| | | | 1,3-oxazolidin-2-one | | | | |
| 314 | 18.0 | nM | 3-[(2′,4′- | 317.34 | 318 | 4.27 | D |
| | | | difluorobiphenyl-4- | | | | |
| | | | yl)methyl]-5,5- | | | | |
| | | | dimethyl-1,3- | | | | |
| | | | oxazolidin-2-one | | | | |
| 315 | 41.4 | nM | 3-[(2′,4′- | 317.34 | 318 | 4.33 | D |
| | | | difluorobiphenyl-4- | | | | |
| | | | yl)methyl]-5-ethyl- | | | | |
| | | | 1,3-oxazolidin-2-one | | | | |
| 316 | 50.4 | nM | (5R)-3-{[4′-chloro-2′- | 385.77 | 386.1 | 2.95 | E |
| | | | (trifluoromethoxy)biphenyl- | | | | |
| | | | 4-yl]methyl}-5- | | | | |
| | | | methyl-1,3- | | | | |
| | | | oxazolidin-2-one | | | | |
| 317 | 36.4 | nM | (5R)-5-methyl-3-{4- | 293.37 | 294.2 | 2.73 | E |
| | | | [(E)-2- | | | | |
| | | | phenylvinyl]benzyl}- | | | | |
| | | | 1,3-oxazolidin-2-one | | | | |
| 318 | 111 | nM | (5R)-5-methyl-3-[4- | 295.38 | 296.1 | 2.82 | E |
| | | | (2- | | | | |
| | | | phenylethyl)benzyl]- | | | | |
| | | | 1,3-oxazolidin-2-one | | | | |

TABLE 1A

| Ex | IUPACNAME | ¹H NMR data |
|---|---|---|
| 48 | 3-[4-(1- | ¹HNMR (CDCl₃) 400 MHz L 7.15 (2H, d), |
| | cyclohexylethoxy)benzyl]- | 6.83 (2H, d), 4.61-4.54 (1H, m), 4.32 (2H, |
| | 5-methyl-1,3-oxazolidin-2- | dd), 4.09 (1H, quint), 3.46 (1H, t), 2.94 (1H, |
| | one | dd), 1.90 (1H br d), 1.74 (3H, br d), 1.66 (1H, |
| | | br d), 1.55 (1H, m, partially obscured by |
| | | water peak), 1.36 (3H, d), 1.22 (3H, d), |
| | | 1.25-1.00 (5H, m). |
| 52 | 3-[4-(1- | 1H NMR (CDCl3) 400 MHz L 7.19 (2H, d); |
| | cyclohexylethoxy)benzyl]- | 6.82 (2H, d); 4.46 (2H, s), 4.23 (2H, t); |
| | 1,3-oxazinan-2-one | 4.13-4.05 (1H, m); 3.19 (2H, t); 2.00-1.91 (2H, m); |
| | | 1.74 (1H, br d); 1.66 (3H, br d); |
| | | 1.60-1.51 (1H, m); 1.26-1.00 (5H, m); 1.21 (3H, d) |
| 57 | (5R)-3-[(4′- | ¹H NMR (CDCl₃) 400 MHz L 1.36 (d, 6H), |
| | isopropoxybiphenyl-4- | 1.40 (d, 3H), 3.01 (dd, 1H), 3.53 (t, 1H), |
| | yl)methyl]-5-methyl-1,3- | 4.44 (dd, 2H), 4.60 (m, 2H), 6.95 (d, 2H), 7.31 (d, |
| | oxazolidin-2-one | 2H), 7.49 (d, 2H), 7.53 (d, 2H) |
| 58 | (5S)-3-[(4′- | ¹H NMR (CDCl₃) 400 MHz L 1.36 (d, 6H), |
| | isopropoxybiphenyl-4- | 1.40 (d, 3H), 3.01 (dd, 1H), 3.53 (t, 1H), |
| | yl)methyl]-5-methyl-1,3- | 4.44 (dd, 2H), 4.60 (m, 2H), 6.95 (d, 2H), 7.31 (d, |
| | oxazolidin-2-one | 2H), 7.49 (d, 2H), 7.53 (d, 2H) |
| 59 | (5S)-3-[(4′-ethoxybiphenyl- | ¹H NMR (CDCl₃) 400 MHz L 1.39 (d, 3H), |
| | 4-yl)methyl]-5-methyl-1,3- | 1.44 (t, 3H), 3.01 (dd, 1H), 3.52 (t, 1H), |
| | oxazolidin-2-one | 4.07 (dd, 2H), 4.44 (dd, 2H), 4.62 (m, 1H), |
| | | 6.96 (d, 2H), 7.31 (d, 2H), 7.50 (d, 2H), 7.53 (d, |
| | | 2H) |
| 60 | (5R)-3-[(4′-ethoxybiphenyl- | ¹H NMR (CDCl₃) 400 MHz L 1.39 (d, 3H), |
| | 4-yl)methyl]-5-methyl-1,3- | 1.44 (t, 3H), 3.01 (dd, 1H), 3.52 (t, 1H), |
| | oxazolidin-2-one | 4.07 (dd, 2H), 4.44 (dd, 2H), 4.62 (m, 1H), |
| | | 6.96 (d, 2H), 7.31 (d, 2H), 7.50 (d, 2H), 7.53 (d, |
| | | 2H) |
| 61 | (5R)-3-[(4′-fluorobiphenyl- | ¹H NMR (CDCl₃) 400 MHz L 1.40 (d, 3H), |
| | 4-yl)methyl]-5-methyl-1,3- | 3.01 (dd, 1H), 3.53 (t, 1H), 4.45 (dd, 2H), |
| | oxazolidin-2-one | 4.64 (m, 1H), 7.12 (t, 2H), 7.34 (d, 2H), |
| | | 7.54 (m, 4H) |
| 62 | (5S)-3-[(4′-fluorobiphenyl- | ¹H NMR (CDCl₃) 400 MHz L 1.40 (d, 3H), |
| | 4-yl)methyl]-5-methyl-1,3- | 3.01 (dd, 1H), 3.53 (t, 1H), 4.45 (dd, 2H), |
| | oxazolidin-2-one | 4.64 (m, 1H), 7.12 (t, 2H), 7.34 (d, 2H), |
| | | 7.54 (m, 4H) |
| 63 | (5S)-5-methyl-3-{[4′- | ¹H NMR (CDCl₃) 400 MHz L 1.40 (d, 3H), |
| | (trifluoromethyl)biphenyl-4- | 3.02 (dd, 1H), 3.55 (t, 1H), 4.47 (dd, 2H), |
| | yl]methyl}-1,3-oxazolidin- | 4.65 (m, 1H), 7.38 (d, 2H), 7.58 (d, 2H), |
| | 2-one | 7.58-7.71 (m, 4H) |
| 64 | (5R)-5-methyl-3-{[4′- | ¹H NMR (CDCl₃) 400 MHz L 1.41 (d, 3H), |
| | (trifluoromethyl)biphenyl-4- | 3.02 (dd, 1H), 3.55 (t, 1H), 4.47 (dd, 2H), |
| | yl]methyl}-1,3-oxazolidin- | 4.65 (m, 1H), 7.38 (d, 2H), 7.58 (d, 2H), |
| | 2-one | 7.60-7.71 (m, 4H) |
| 67 | (5R)-5-methyl-3-{4-[(1R)- | ¹H NMR (CDCl₃) 400 MHz L 1.35 (d, 3H), |
| | 1-phenylethoxy]benzyl}- | 1.62 (d, 3H), 2.91 (t, 1H), 3.42 (t, 1H), |
| | 1,3-oxazolidin-2-one | 4.29 (dd, 2H), 4.56 (quintet, 1H), 5.28 (q, 1H), |
| | | 6.82 (d, 2H), 7.09 (d, 2H), 7.24-7.38 (m, 5H) |
| 87 | (5R)-3-[(2,2-dimethyl-3,4- | ¹H NMR (CDCl₃) 400 MHz L 1.32 (s, 6H), |
| | dihydro-2H-chromen-6- | 1.39 (d, 3H), 1.79 (t, 2H), 2.75 (t, 2H), |
| | yl)methyl]-5-methyl-1,3- | 2.99 (dd, 1H), 3.51 (t, 1H), 4.30 (dd, 2H), 4.63 (m, |
| | oxazolidin-2-one | 1H), 6.74 (d, 1H), 6.96 (m, 2H) |
| 187 | (5R)-3-[(3′,5′- | ¹H NMR (CDCl₃) 400 MHz L 1.41 (d, 3H), |
| | dichlorobiphenyl-4- | 3.01 (dd, 1H), 3.53 (t, 1H), 4.46 (dd, 2H), |
| | yl)methyl]-5-methyl-1,3- | 4.64 (sextet, 1H), 7.34 (d, 2H), 7.37 (s, 1H), |
| | oxazolidin-2-one | 7.44 (s, 2H), 7.52 (d, 2H) |
| 188 | 1-[4-(1- | ¹H-NMR (400 Hz, CDCl₃) L: 7.10 (d, 2H), |
| | cyclohexylethoxy)benzyl]- | 6.80 (d, 2H), 4.54 (s, 2H), 4.06 (m, 1H), |
| | 4-ethylpiperazin-2-one | 3.85 (m, 2H), 3.20-3.40 (m, 3H), 3.17 (m, 2H), |
| | | 1.90 (d, 1H), 1.60-1.75 (m, 4H), 1.54 (m, |
| | | 1H), 1.36 (m, 3H), 1.20 (d, 3H), |
| | | 1.10-1.25 (m, 6H). |
| 203 | (5R)-3-[4- | ¹H-NMR (400 Hz, CDCl₃) L 1.04 (dq, 2H), |
| | (cyclohexylmethoxy)benzyl]- | 1.15-1.35 (m, 3H), 1.37 (d, 3H), |
| | 5-methyl-1,3-oxazolidin- | 1.69-1.87 (m, 6H), 2.94 (dd, 1H), 3.46 (t, 1H), 3.73 (d, |
| | 2-one | 2H), 4.34 (q, 2H), 4.58 (sextet, 1H), 6.86 (d, |
| | | 2H), 7.18 (d, 2H) |
| 204 | (5R)-5-methyl-3-[4-(3- | ¹H-NMR (400 Hz, CDCl₃) L 1.37 (d, 3H), |
| | phenylpropoxy)benzyl]- | 2.07-2.14 (m, 2H), 2.81 (t, 2H), 2.94 (dd, |
| | 1,3-oxazolidin-2-one | 1H), 3.46 (t, 1H), 3.95 (t, 2H), 4.34 (dd, 2H), |
| | | 4.59 (sextet, 1H), 6.86 (d, 2H), 7.17-7.31 (m, |
| | | 7H) |
| 210 | (5R)-3-{[6-(4-chloro-2- | ¹H-NMR (400 Hz, CD₃OD) L 1.38 (d, 3H), |
| | fluorophenyl)pyridin-3- | 3.15 (dd, 1H), 3.68 (t, 1H), 4.51 (dd, 2H), |
| | yl]methyl}-5-methyl-1,3- | 4.71 (m, 1H), 7.34-7.37 (m, 2H), |
| | oxazolidin-2-one | 7.79-7.89 (m, 3H), 8.63 (d, 1H) |
| 228 | (5R)-3-(3-fluoro-4-{[2- | ¹H-NMR (400 Hz, CD₃OD) L 1.34 (d, 3H), |
| | fluoro-4- | 3.04 (t, 1H), 3.59 (t, 1H), 4.34 (dd, 2H), |
| | (trifluoromethyl)benzyl]oxy}benzyl)- | 4.66 (m, 1H), 5.28 (s, 2H), 7.05-7.11 (m, 2H), |
| | 5-methyl-1,3- | 7.18 (dt, 1H), 7.49-7.55 (m, 2H), 7.76 (t, 1H) |
| | oxazolidin-2-one | |
| 238 | (5R)-5-methyl-3-{[4′- | ¹H-NMR (400 Hz, CDCl₃) L 1.41 (d, 3H), |
| | (trifluoromethoxy)biphenyl- | 3.02 (dd, 1H), 3.54 (t, 1H), 4.46 (dd, 2H), |
| | 4-yl]methyl}-1,3- | 4.60-4.69 (m, 1H), 7.29 (d, 2H), 7.36 (d, 2H), |
| | oxazolidin-2-one | 7.54 (d, 2H), 7.58 (d, 2H) |
| 255 | (5R)-5-methyl-3-({6-[4- | ¹H-NMR (400 Hz, CDCl₃) L 1.41 (d, 3H), |
| | (trifluoromethoxy)phenyl]pyridin- | 3.04 (dd, 1H), 3.56 (t, 1H), 4.49 (dd, 2H), |
| | 3-yl}methyl)-1,3- | 4.62-4.69 (m, 1H), 7.28-7.36 (m, 2H), |
| | oxazolidin-2-one | 7.72-7.78 (m, 2H), 8.03 (d, 2H), 8.60 (s, 1H) |
| 291 | (5R)-3-[(3′-chloro-2,2′- | ¹H-NMR (400 Hz, CD₃OD) L 1.39 (d, 3H), |
| | difluorobiphenyl-4- | 3.14 (dd, 1H), 3.67 (t, 1H), 4.47 (dd, 2H), |
| | yl)methyl]-5-methyl-1,3- | 4.67-4.76 (m, 1H), 7.17-7.54 (m, 6H) |
| | oxazolidin-2-one | |

O. Biological Protocols

In Vitro Assays

Procedure for mGluR2Potentiator Screen NLB Methods EC10-EC20 Challenge

Cell Culture and Plating:

Cells used for this screen are HEK cells stably transfected with the mGluR2 receptor (metabotropic glutamate receptor 2) and the GL15 G protein. Clones were identified by functional activity (FLIPR). Cells are grown in growth media containing: DMEM High Glucose with Glutamine and Na Pyruvate (GIBCO), 10% (v/v) Heat inactivate FBS (GIBCO), G418 500 ug/ml (from 50 mg/ml stock) (GIBCO) and Blasticidin 3 ug/ml (from 5 mg/ml stock made in H2O) (Invitrogen).

2 days before the assay cell are trypsinized with 0.25% trypsin/EDTA (GIBCO), spun down at 1000 rpm for 5 minutes, resuspended in growth media and plated on polystyrene 384 well black wall/clear bottom poly-D-lysine coated plates at a density of approximately 18,000 cells/well in a volume of 50 μL per well. One day before the assay the growth media is removed from the plates by flicking, and replaced with media containing DMEM High Glucose without Glutamine and Na Pyruvate (GIBCO) and 10% (v/v) dialyzed FBS (GIBCO). The reason for the removal of glutamine the day before the assay is to minimize the amount of glutamate that will be present during the assay, as endogenous glutamate released from the cells can reduce the fluorescent response and interfere with the FLIPR screen.

FLIPR Methods and Data Analysis:

On the day of the assay, the FLIPR assay is performed using the following methods:

Assay buffer:

| Compound | g/L | MW | [concentration] |
|---|---|---|---|
| NaCl | 8.47 | 58.44 | 145 mM |
| Glucose | 1.8 | 180.2 | 10 mM |
| KCl | .37 | 74.56 | 5 mM |
| MgSO₄ | 1 ml 1M Stock | 246.48 | 1 mM |
| HEPES | 2.38 | 238.3 | 10 mM |
| CaCl₂ | 2 ml 1M Stock | 110.99 | 2 mM |

The pH is adjusted to 7.4 with 1M NaOH. Prepare a 2 mM (approx.) stock solution of Fluo-4, am (Molecular Probes) dye in DMSO-22 μl DMSO per 50 ug vial (440 μL per 1 mg vial). Make a 1 mM (approx.) flou-4, PA working solution per vial by adding 22 μl of 20% pluronic acid (PA) (Molecular Probes) in DMSO to each 50 ug vial (440 μL per 1 mg vial). Prepare a 250 mM Probenecid (Sigma) stock solution by dissolving 0.71g into 5 ml 1N NaOH and 5 ml assay buffer (for each liter of assay wash buffer). Make 4 uM (approx.) dye incubation media by adding 2 50 ug vials per 11 ml DMEM high glucose without glutamine (220 ml per 1 mg vial). Add 110 μL probenecid stock per 11 ml (2.5 mM final [concentration]). To the dye media add 3 units/ml of glutamic-pyruvic transaminase (GPT, Sigma) and 3 mM Na Pyruvate. The assay has worked with dye concentrations from 2 uM to 8 uM dye as well. To the assay buffer from drug preparation, add 1.83 mls DMSO and 400 μL 15.8% P104 (from New Leads biology) per liter for final concentrations of 0.18% DMSO and 0.006% P104. To the assay buffer for cell washing, add probenecid in the same manner and concentration that was used for the dye media.

Remove growth media from cell plates by flicking. Add 50 μl/well dye solution. Incubate 1 hour at 37° C. and 5% CO₂. Remove dye solution and wash 3 times with assay buffer+probenecid (100 μl probenecid stock per 10 ml buffer), leaving 30 μL/well assay buffer. Wait at least 10-15 minutes. Compounds and agonist challenge additions are performed with the FLIPR. The 1^{st} addition is for test compounds, which are added as 15 μL of 4× [concentration] of potentiator. The second 2^{nd} addition is 15 μL of 4× [concentration] of agonist or challenge. This achieves 1× concentration of all compounds only after 2^{nd} addition. The 1^{st} and 2^{nd} additions are performed separately using the FLIPR, which give 2 different data files. Compounds are pretreated at least 30 minutes before agonist addition.

Results are analyzed by dividing the peak fluorescent value of the FLIPR response by the time point after agonist addition to achieve a ratio response. The ratios are then analyzed by curve fitting programs. Since potent compounds can give an inverted U dose response curve (due to effects on endogenous glutamate by the potentiators), points are deleted at concentrations higher than the concentration that gives the maximum effect. Maximum values for dose response curves (forced fitting) are derived from standards on the plate.

Compound Preparation and Glutamate Challenge:

Compounds are delivered as 10 mM DMSO stocks or as powders. Powders are solubilized in DMSO at 10 mM (as solubility allows). Compounds are sonicated in a heated water bath (35-40° C.) for at least 20 minutes. Compounds are then added to assay drug buffer as 40 μL top [concentration] (4× the 10 uM top screening concentration).

In order to test compounds against an EC10 to EC20 concentration of glutamate, multiple glutamate challenge plates for the 2^{nd} FLIPR addition are prepared. The best challenge for a particular assay is determined by examining the glutamate dose response and 1-4 test plates.

EC₅₀ values of the compounds of the invention are preferably 15 micromolar or less, more preferably 1 micromolar or less, even more preferably 100 nanomolar or less.

When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations to the invention, the scope of which is defined by the appended claims.

1. A compound of formula I: 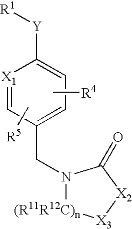
or a pharmaceutically acceptable salt thereof,
wherein Y is a bond, NR²², or O;
wherein, when Y is NR²² or O,
R¹ is alkyl, aryl, heteroaryl, heterocycloalkyl, or cycloalkyl each of which is optionally substituted with one, two, three or four R⁴¹, wherein each R⁴¹ is independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴¹ alkyl, heterocycloalkyl, cycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², NR¹⁰¹C(O)R¹⁰³, and C(O)R¹⁰³;
or, when R¹ is aryl, heteroaryl, cycloalkyl or heterocycloalkyl, two R⁴¹ substituents bonded to adjacent carbon atoms of R¹, together with the adjacent carbon atoms, form a heterocylic or carbocyclic ring which is optionally substituted with one or more R¹⁰;
wherein each R¹⁰ is independently selected from the group consisting of hydrogen, —CN, halogen, —C(O)R¹⁰¹, —C(O)NR¹⁰¹R¹⁰², —NR¹⁰¹R¹⁰², —OR¹⁰¹or —R¹⁰¹;
and when Y is a bond,
R¹ is either
(a) aryl, heteroaryl, heterocycloalkyl, or cycloalkyl wherein R¹ is optionally substituted with one, two, three or four R⁴¹, wherein each R⁴¹ is independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴¹ alkyl, heterocycloalkyl, cycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰² NR¹⁰¹S(O)₂R¹⁰³, —OC(O)R¹⁰³, —(O)OR¹⁰³, —C(O)NR¹⁰¹R¹⁰², NR¹⁰¹C(O)R¹⁰³, and C(O)R¹⁰³;
or wherein, when R¹ is aryl, heteroaryl, cycloalkyl or heterocycloalkyl, two R⁴¹ substituents bonded to adjacent carbon atoms of R¹, together with the adjacent carbon atoms, form a heterocyclic or carbocyclic ring which is optionally substituted with one or more R¹⁰;
or
(b) alkyl or alkenyl substituted with one, two, three or four R⁴² and further optionally substituted with halogen, wherein each R⁴² is independently selected from the group consisting of cyano, —OR¹⁰¹, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴² heterocycloalkyl, cycloalkyl, cycloalkenyl, aryl or heteroaryl is optionally substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², NR¹⁰¹C(O)R¹⁰³, and C(O)R¹⁰³;
X₁ is CR⁶ or N;
n is 1 or 2;
X₂ is O or CR⁷R⁸;
X₃ is NR²³, O, or CR²R³;
with the proviso that if X₂ is O, X₃ is CR²R³, and
with the proviso that if X₂ is CR⁷R⁸, X³ is NR²³ or O;
wherein
each of R² and R³ is independently selected from the group consisting of hydrogen, alkyl, aryl, heteroaryl, heterocycloalkyl, and cycloalkyl wherein the R² or R³ alkyl, aryl, heteroaryl, heterocycloalkyl, or cycloalkyl is optionally substituted with one, two, three or four R⁴³, wherein each R⁴³ is independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴³ alkyl, heterocycloalkyl, cycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², NR¹⁰¹C(O)R¹⁰³, and C(O)R¹⁰³;
q is 0, 1 or 2;
or R² and R³ taken together with the carbon that R² and R³ are attached to form a carbocyclic or heterocyclic ring, optionally substituted with one, two, three or four R⁴³;
each R¹⁰¹ and each R¹⁰² is independently selected from the group consisting of hydrogen, alkyl, alkenyl, alkynyl, cycloalkyl, aryl, heterocycloalkyl and heteroaryl;
wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;
R¹⁰³ is independently selected from the group consisting of alkyl, alkenyl, cycloalkyl, aryl, heterocycloalkyl and heteroaryl and is optionally substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;
R²² is hydrogen, alkyl, heterocycloalkyl, or cycloalkyl wherein the R²² alkyl, heterocycloalkyl, or cycloalkyl is optionally substituted with one, two, three or four alkyl, heterocycloalkyl, cycloalkyl, aryl, heteroaryl, halogen, or OR¹⁰¹, wherein the heterocycloalkyl, cycloalkyl, aryl, or heteroaryl substituent on R²² is optionally substituted with alkyl, cycloalkyl, halogen or OR¹⁰¹;
R²³ is alkyl, heterocycloalkyl, aryl, heteroaryl, or cycloalkyl wherein R²³ is optionally substituted with one, two, three or four alkyl, heterocycloalkyl, cycloalkyl, aryl, heteroaryl, halogen, or OR¹⁰¹, wherein the heterocycloalkyl, cycloalkyl, aryl, or heteroaryl substituent on R²³ is optionally substituted with alkyl, cycloalkyl, halogen or OR¹⁰¹;
each R⁷, R⁸, R¹¹ or R¹² is independently hydrogen, alkyl, aryl, heteroaryl, heterocycloalkyl, or cycloalkyl, wherein the R⁷, R⁸, R¹¹ or R¹² alkyl, aryl, heteroaryl, heterocycloalkyl, or cycloalkyl is optionally substituted with one, two, three or four groups independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³;
or when n is 2, the R¹¹ and R¹² taken together with the carbon atoms interconnecting them form a 5-7 membered carbocyclic or heterocyclic ring that is optionally substituted with one or two groups independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —C(O)NR¹⁰¹R¹⁰², —NR¹⁰¹R¹⁰², NR¹⁰¹C(O)R¹⁰³, and —NR¹⁰¹S(O)₂R¹⁰³;
R⁴, R⁵ and R⁶ are each independently selected from the group consisting of hydrogen, halogen, alkyl optionally substituted with one or more halogens, alkoxy optionally substituted with one or more halogens, and cyano;
or if X₂ is O and X₃ is CR²R³, and two of the substituents R⁴, R⁵ and R⁶ are bonded to adjacent carbon atoms, the two of the substituents R⁴, R⁵ and R⁶ together with the adjacent carbon atoms form a heterocyclic or carbocyclic ring which is optionally substituted with one or more R¹⁰;
or, if X₂ is CR⁷R⁸ and X₃ is NR²³, and two of the substituents R⁴, R⁵ and R⁶ are bonded to adjacent carbon atoms, the two of the substituents R⁴, R⁵ and R⁶ together with the adjacent carbon atoms form a carbocylic or aliphatic heterocyclic ring which is optionally substituted with one or more R¹⁰;
or R⁶ and R¹ taken together with the atoms that R⁶ and R⁴¹ are attached to form a carbocyclic or heterocyclic ring that is optionally substituted with alkyl, cycloalkyl, halogen, or OR¹⁰¹;
or R⁶ and R⁴¹ taken together with the atoms that R⁶ and R⁴¹ are attached to form a carbocylic or heterocyclic ring that is optionally substituted with alkyl, cycloalkyl, halogen, or OR¹⁰¹.

2. The compound of claim 1, or a pharmaceutically acceptable salt thereof, wherein n=1.

3. The compound of claim 1, or a pharmaceutically acceptable salt thereof, wherein X₃ is CR²R³ wherein one or both of R² and R³ are alkyl.

4. The compound of claim 1, or a pharmaceutically acceptable salt thereof, wherein X₃ is CR²R³ wherein one of R² and R³ is hydrogen and the other of R² and R³ is alkyl or aryl.

5. The compound of claim 1, or a pharmaceutically acceptable salt thereof, wherein R¹ is cyclobutyl, cyclopentyl optionally fused to a benzene ring, cyclohexyl optionally fused to a benzene ring, cycloheptyl, decalinyl, norbornyl, morpholinyl, or tetrahydropyranyl, optionally substituted as in the compound of claim 1.

6. The compound of claim 1, or a pharmaceutically acceptable salt thereof, wherein R¹ is phenyl which may be substituted by one or two substituents R⁴¹ independently selected from the group consisting of halogen, cyano, alkyl optionally substituted with halogen, alkoxy optionally substituted with halogen, carboxyalkyl, alkylcarbonyl, and cycloalkoxy optionally substituted with alkyl or halogen.

7. The compound of claim 6, or a pharmaceutically acceptable salt thereof, wherein —Y— is a bond.

8. The compound of claim 1, or a pharmaceutically acceptable salt thereof, wherein R¹ is alkyl substituted with one, two, three or four R⁴², wherein each R⁴² is independently selected from the group consisting of —OR¹⁰¹, cycloalkyl, cycloalkenyl, heterocycloalkyl, aryl, heteroaryl, —C(O)R¹⁰¹, —C(O)OR¹⁰¹, —C(O)NR¹⁰¹R¹⁰²,—NR¹⁰¹R¹⁰², NR¹⁰¹C(O)R¹⁰³, and —NR¹⁰¹S(O)₂R¹⁰³ wherein each of the R⁴² alkyl, heterocycloalkyl, cycloalkyl, aryl or heteroaryl is optionally independently substituted as in claim 1.

9. The compound of claim 1, or a pharmaceutically acceptable salt thereof, wherein the group

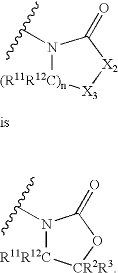

10. The compound of claim 1, or a pharmaceutically acceptable salt thereof, wherein the compound of claim 1 has the following formula, with the absolute stereochemistry as shown:

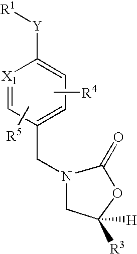

11. The compound of claim 10, or a pharmaceutically acceptable salt thereof, wherein R³ is methyl optionally substituted as in claim 1.

12. The compound of claim 10, or a pharmaceutically acceptable salt thereof, wherein R¹ is phenyl optionally substituted with one, two, three or four R⁴¹, wherein each R⁴¹ is independently selected from the group consisting of halogen, —CN, —OR¹⁰¹, alkyl, cycloalkyl, heterocycloalkyl, aryl, heteroaryl, —C(O)R¹⁰¹, —C(O)OR¹⁰¹, and —NR¹⁰¹R¹⁰², wherein each of the R⁴¹ alkyl, heterocycloalkyl, cycloalkyl, aryl or heteroaryl is optionally independently substituted as in claim 1.

13. The compound of claim 1, or a pharmaceutically acceptable salt thereof, wherein the compound of claim 1 has the following formula, with the absolute stereochemistry as shown:

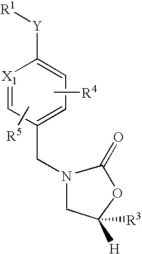

14. The compound of claim 13, or a pharmaceutically acceptable salt thereof, wherein R³ is methyl optionally substituted as in claim 1.

15. The compound of claim 13, or a pharmaceutically acceptable salt thereof, wherein R¹ is phenyl optionally substituted with one, two, three or four R⁴¹, wherein each R⁴¹ is independently selected from the group consisting of halogen, —CN, —OR¹⁰¹, alkyl, cycloalkyl, heterocycloalkyl, aryl, heteroaryl, —C(O)R¹⁰¹, —C(O)OR¹⁰¹, and —NR¹⁰¹R¹⁰², wherein each of the R⁴¹ alkyl, heterocycloalkyl, cycloalkyl, aryl or heteroaryl is optionally independently substituted as in claim 1.

16. A compound selected from the group consisting of the compounds disclosed in Table 1 herein, and pharmaceutically acceptable salts thereof.

17. A method of treating a condition selected from the group consisting of cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder in a mammal, comprising administering in an amount effective to treat the condition a compound of claim 1 or a pharmaceutically acceptable salt thereof to the mammal.

18. A method according to claim 17 further comprising administering to the mammal a metabotropic glutamate receptor agonist.

19. A pharmaceutical composition comprising a compound of claim 1 or a pharmaceutically acceptable salt thereof in a therapeutically effective amount and a pharmaceutically acceptable carrier.

20. The composition of claim 19, further comprising a metabotropic glutamate receptor agonist.
